SEQUENCE LISTING (26 sequences)

SEQ ID NO: 1 - protein, 9 aa, Artificial Sequence
  Description of Artificial Sequence Synthetic peptide
DXNDNAPXF

SEQ ID NO: 2 - DNA, 2721 bp, Homo sapiens
atgtgccggatagcgggagcgctgcggaccctgctgccgctgctggcggccctgcttcaggcgtctgtagaggcttctggtgaaatcgcattatgcaagactggatttcctgaagatgtttacagtgcagtcttatcgaaggatgtgcatgaaggacagcctcttctcaatgtgaagtttagcaactgcaatggaaaaagaaaagtacaatatgagagcagtgagcctgcagattttaaggtggatgaagatggcatggtgtatgccgtgagaagctttccactctcttctgaacatgccaagttcctgatatatgcccaagacaaagagacccaggaaaagtggcaagtggcagtaaaattgagcctgaagccaaccttaactgaggagtcagtgaaggagtcagcagaagttgaagaaatagtgttcccaagacaattcagtaagcacagtggccacctacaaaggcagaagagagactgggtcatctctccaatcaacttgccagaaaactccaggggaccttttcctcaagagcttgtcaggatcaggtctgatagagataaaaacctttcactgcggtacagtgtaactgggccaggagctgaccagcctccaactggtatcttcattatcaaccccatctcgggtcagctgtcggtgacaaagcccctggatcgcgagcagatagcccggtttcatttgagggcacatgcagtagatattaatggaaatcaagtggagaaccccattgacattgtcatcaatgttattgacatgaatgacaacagacctgagttcttacaccaggtttggaatgggacagttcctgagggatcaaagcctggaacatatgtgatgaccgtaacagcaattgatgctgacgatcccaatgccctcaatgggatgttgaggtacagaatcgtgtctcaggctccaagcaccccttcacccaacatgtttacaatcaacaatgagactggtgacatcatcacagtggcagctggacttgatcgagaaaaagtgcaacagtatacgttaataattcaagctacagacatggaaggcaatcccacatatggcctttcaaacacagccacggccgtcatcacagtgacagatgtcaatgacaatcctccagagtttactgccatgacgttttatggtgaagttcctgagaacagggtagacatcatagtagctaatctaactgtgaccgataaggatcaaccccatacaccagcctggaacgcagtgtacagaatcagtggcggagatcctactggacggttcgccatccagaccgaccaaaacagcaacgacgggttagtcaccgtggtcaaaccaatcgactttgaagcaaataggatgtttgtccttactgttgctgcagaaaatcaagtgccattagccaagggaattcagcacccgcctcagtcaactgcaaccatgtctgttacagttattgacgtaaatgaaaacccttattttgcccccaatcctaagatcattcgccaagaagaagggcttcatgccggtaccatgttgacaacattcactgctcaggacccagatcgatatatgcagcaaaatattagatacactaaattatctgatcctgccaattggctaaaaatagatcctgtgaatggacaaataactacaattgctgttttggaccgagaatcaccaaatgtgaaaaacaatatatataatgctactttccttgcttctgacaatggaattcctcctatgagtggaacaggaacgctgcagatctatttacttgatattaatgacaatgcccctcaagtgttacctcaagaggcagagacttgcgaaactccagaccccaattcaattaatattacagcacttgattatggcattgatccaaatgctggaccatttgcttttgatcttcctttatctccagtgactattaagagaaattggaccatcactcggcttaatggtgattttgctcagcttaatttaaagataaaatttcttgaagctggtatctatgaagttcccatcataatcacagattcgggtaatcctcccaaatcaaatatttccatactgcgcgtgaaggtttgccagtgtgactccaacggggactgcacagatgtggacaggattgtgggtgcggggcttggcaccggtgccatcattgccatcctgctctgcatcatcatcctgcttatccttgtgctgatgtttgtggtatggatgaaacgccgggataaagaacgccaggccaaacaacttttaattgatccagaagatgatgtaagagataatattttaaaatatgatgaagaaggtggaggagaagaagaccaggactatgacttgagccagctgcagcagcctgacactgtggagcctgatgccatcaagcctgtgggaatccgacgaatggatgaaagacccatccacgctgagccccagtatccggtccgatctgcagccccacaccctggagacattggggacttcattaatgagggccttaaagcggctgacaatgaccccacagctccaccatatgactccctgttagtgtttgactatgaaggcagtggctccactgctgggtccttgagctcccttaattcctcaagtagtggtggtgagcaggactatgattacctgaacgactgggggccacggttcaagaaacttgctgacatgtatggtggaggtgatgactga

SEQ ID NO: 3 - DNA, 64 bp, Artificial Sequence
  Description of Artificial Sequence Synthetic oligonucleotide
gatccccgatgtttacagcgcagtctttcaagagaagactgcgctgtaaacatctttttggaaa

SEQ ID NO: 4 - DNA, 61 bp, Artificial Sequence
  Description of Artificial Sequence Synthetic oligonucleotide
agcttttccaaaaagatgtttacagcgcagtctcttgaaagactgcgctgtaaacatcggg

SEQ ID NO: 5 - DNA, 64 bp, Artificial Sequence
  Description of Artificial Sequence Synthetic oligonucleotide
gatccccgctatcgctacgtgtaagtttcaagagaacttacacgtagcgatagctttttggaaa

SEQ ID NO: 6 - DNA, 64 bp, Artificial Sequence
  Description of Artificial Sequence Synthetic oligonucleotide
agcttttccaaaaagctatcgctacgtgtaagttctcttgaaacttacacgtagcgatagcggg

SEQ ID NO: 7 - DNA, 19 bp, Homo sapiens
gactggatttcctgaagat

SEQ ID NO: 8 - DNA, 19 bp, Homo sapiens
gactggatttcctgaagat

SEQ ID NO: 9 - DNA, 19 bp, Artificial Sequence
  Description of Artificial Sequence Synthetic oligonucleotide
ggtgtggctgtcagtgctc

SEQ ID NO: 10 - DNA, 30 bp, Artificial Sequence
  Description of Artificial Sequence Synthetic oligonucleotide
gtaattgcggcaggaggaattgtttctgtc

SEQ ID NO: 11 - DNA, 30 bp, Artificial Sequence
  Description of Artificial Sequence Synthetic oligonucleotide
gacagaaacaattcctcctgccgcaattac

SEQ ID NO: 12 - DNA, 18 bp, Artificial Sequence
  Description of Artificial Sequence Synthetic oligonucleotide
ggtgtggctgtcagtgct

SEQ ID NO: 13 - DNA, 30 bp, Artificial Sequence; identical to SEQ ID NO: 10
  Description of Artificial Sequence Synthetic oligonucleotide

SEQ ID NO: 14 - DNA, 20 bp, Mus musculus
ctgctttctgttgagaggct

SEQ ID NO: 15 - RNA, 25 nt, Artificial Sequence
  Description of Artificial Sequence Synthetic oligonucleotide
gggaagacagaacuuguacucaaaa

SEQ ID NO: 16 - RNA, 25 nt, Artificial Sequence
  Description of Artificial Sequence Synthetic oligonucleotide
uuuugaguacaaguucugucuuccc

SEQ ID NO: 17 - RNA, 26 nt, Artificial Sequence
  Description of Artificial Sequence Synthetic oligonucleotide
aaaacucauguucaagacagaagggu

SEQ ID NO: 18 - RNA, 26 nt, Artificial Sequence
  Description of Artificial Sequence Synthetic oligonucleotide
acccuucugucuugaacaugaguuuu

SEQ ID NO: 19 - RNA, 25 nt, Artificial Sequence
  Description of Artificial Sequence Synthetic oligonucleotide
ccaucugaugguguccuaccuaauu

SEQ ID NO: 20 - RNA, 25 nt, Artificial Sequence
  Description of Artificial Sequence Synthetic oligonucleotide
aauuagguaggacaccaucagaugg

SEQ ID NO: 21 - RNA, 25 nt, Artificial Sequence
  Description of Artificial Sequence Synthetic oligonucleotide
gggcucuuggcuugcuauucgaauu

SEQ ID NO: 22 - RNA, 25 nt, Artificial Sequence
  Description of Artificial Sequence Synthetic oligonucleotide
aauucgaauagcaagccaagagccc

SEQ ID NO: 23 - RNA, 25 nt, Artificial Sequence
  Description of Artificial Sequence Synthetic oligonucleotide
ccuuccaccuuuagauaaagagaaa

SEQ ID NO: 24 - RNA, 25 nt, Artificial Sequence
  Description of Artificial Sequence Synthetic oligonucleotide
uuucucuuuaucuaaagguggaagg

SEQ ID NO: 25 - DNA, 20 bp, Artificial Sequence
  Description of Artificial Sequence Synthetic oligonucleotide
agcctctcaacagaaagcag

SEQ ID NO: 26 - DNA, 20 bp, Artificial Sequence
  Description of Artificial Sequence Synthetic oligonucleotide
tatggtacgtgtcgtccttg

(19) United States
(12) Patent Application Publication
(10) Pub. No.: US 2014/0274872 A1
(43) Pub. Date: Sep. 18, 2014

(54) COMPOSITIONS AND TREATMENTS BASED ON CADHERIN MODULATION

(71) Applicant: CODA THERAPEUTICS, INC., SAN DIEGO, CA (US)

(72) Inventors: BRADFORD J. DUFT, RANCHO SANTA FE, CA (US); DAVID L. BECKER, ABBOTS LANGLEY (GB)

(73) Assignee: CODA THERAPEUTICS, INC., SAN DIEGO, CA (US)

(21) Appl. No.: 13/844,553

(22) Filed: Mar. 15, 2013

(51) Int. Cl. A61K 31/713; A61K 45/06; A61K 31/7088; C07H 21/00; C07H 21/02

(52) U.S. Cl. CPC A61K 31/713; C07H 21/00; C07H 21/02; A61K 31/7088; A61K 45/06
USPC 514/1.1; 536/24.5; 514/44 A

(57) ABSTRACT

Anti-cadherin and anti-ZO-1 agents and compositions, and kits containing them for use in the promotion and/or improvement of wound healing and/or tissue repair, and for anti-scarring, anti-inflammatory, anti-fibrosis and anti-adhesion indications.

### CROSS-REFERENCE TO RELATED APPLICATIONS

This application is a non-provisional of and claims the benefit of U.S. Provisional Application Ser. No. 61/616,393, filed on Mar. 27, 2012, the entire contents of which is hereby incorporated by reference.

### TECHNICAL FIELD

The inventions relate compositions and methods that involve modulation of cadherin proteins. These inventions are useful in various contexts, including to promote wound healing and to treat wounds, in particular acute wounds and to wounds that do not heal at expected rates, such as delayed-healing wounds, incompletely healing wounds, chronic wounds, and dehiscent wounds.

### BACKGROUND OF THE INVENTION

The following includes information that may be useful in understanding the present inventions. It is not an admission that any of the information provided herein is prior art, or relevant, to the presently described or claimed inventions, or that any publication or document that is specifically or implicitly referenced is prior art.

In humans and other mammals wound injury triggers an organized complex cascade of cellular and biochemical events that will in most cases result in a healed wound. An ideally healed wound is one that restores normal anatomical structure, function, and appearance on cellular, tissue, organ, and organism levels. Wound healing, whether initiated by trauma, microbes or foreign materials, proceeds via a complex process encompassing a number of overlapping phases, including inflammation, epithelialization, angiogenesis and matrix deposition. Normally, these processes lead to a mature wound and a certain degree of scar formation. Although inflammation and repair mostly occur along a prescribed course, the sensitivity of the process is dependent on the balance of a variety of wound healing molecules, including for example, a network of regulatory cytokines and growth factors.

Cell adhesion and intercellular communication also have a role in wound healing. Cell adhesion involves a cell binding to surface, extracellular matrix, and/or one or more adjacent cells through a variety of cell adhesion molecules such as cadherins, integrins, and selectins. Intercellular communication includes connexin proteins, which form gap junctions. Appropriate cell adhesion is essential not only to maintaining multicellular structure and restoring such structure after injury, but also facilitating intercellular communication via messenger and signal transduction.

Cadherins are a class of type-1 transmembrane proteins involved in binding cells together to form, maintain, and restore tissues. As a family, cadherins are adhesion molecules that mediate Ca²⁺-dependent cell-cell adhesion in all solid tissues of multicellular animals. These proteins modulate a wide variety of processes, including cell polarization and migration. Cadherin-mediated cell-cell junctions form as a result of interaction between extracellular domains of identical cadherins, which protrude from the membranes of adjoining cells. The stability of these adhesive junctions is ensured by binding of the intracellular cadherin domain with the actin cytoskeleton.

The cadherin superfamily includes cadherins, protocadherins, desmogleins, and desmocollins, among others. Structurally, each superfamily member has cadherin repeats, which are extracellular, calcium ion-binding domains. A number of different cadherin isoforms or subtypes exist, and are distributed in a tissue-specific manner in a wide variety of organisms. The different cadherin isoforms or subtypes are designated with a prefix denoting the tissue with which it is normally associated. For example, N-cadherin denotes neural origin, E-cadherin, epithelium, and P-cadherin, placenta. It has been observed that cells expressing a specific cadherin subtype tend to cluster together to the exclusion of other types, both in cell culture and during development, meaning, for example, that cells that express N-cadherin on their surfaces tend to cluster with other N-cadherin-expressing cells. It has also been found that cadherin expression causes morphological changes involving the positional segregation of cells into layers, indicating that cadherins are important in the sorting of different cell types during morphogenesis, histogenesis, and regeneration. They may also be involved in the regulation of tight and gap junctions, and in the control of intercellular spacing.

Structurally, members of the cadherin superfamily comprise a number of domains: a signal sequence; a propeptide of around 130 residues; a single transmembrane domain; at least one extracellular domain containing a cadherin repeat motif; and an N-terminal cytoplasmic domain. Classical cadherin proteins have an extracellular domain that includes five tandemly repeated extracellular cadherin domains, four of which are cadherin repeats, and the fifth of which contains four conserved cysteines. A “cadherin repeat” is an independently folding peptide of approximately 110 amino acid residues that contains motifs with the conserved sequences DRE, DXNDNAPXF (SEQ ID NO:1), and DXD. Studies of crystallized extracellular cadherin domains reveal that multiple cadherin domains form Ca²⁺-dependent rod-like structures with a conserved calcium-binding pocket at the domain-domain interface. Cadherins depend on calcium for function, as calcium ions bind to specific residues in each cadherin repeat to ensure proper folding, to confer rigidity upon the extracellular domain, and to prevent protease digestion.

Cadherin proteins are the primary extracellular components of “adherens junctions” (also termed “intermediate junctions” or “belt desmosomes”) between cells in vivo. Adherens junctions are protein complexes that occur at cell-cell junctions in epithelial tissues, and are defined as cell junctions that have a cytoplasmic face linked to an actin cytoskeleton. Adherens junctions are usually more basal than “tight junctions”. They can appear as bands encircling a cell (“zonula adherens”) or as spots of attachment to the extracellular matrix (“adhesion plaques”). At adherens junctions, the intercellular portion of a cadherin protein interacts with a catenin or vinculin subunit, through which an actin filament projects. The extracellular domain of a cadherin protein forms a homodimer with the extracellular domain of a cadherin protein of an adjacent cell in a calcium dependent manner.

Like adherens junctions, gap junctions are cell membrane structures; however, gap junctions facilitate direct cell-cell communication. A gap junction channel is formed of two connexons (hemichannels), each composed of six connexin subunits, which allows direct connection between the cytoplasm of adjoining cells when the hemichannels are in an “open”, as opposed to a “closed” configuration. When the connexons forming a gap junction channel are “open”, molecules (e.g., ions, signaling molecules, etc.) can move from one cell to another. Each hexameric connexon docks with a connexon in the opposing membrane to form a single gap junction. Gap junction channels are reportedly found throughout the body. Tissue such as the corneal epithelium, for example, has six to eight cell layers, yet expresses different gap junction channels in different layers with connexin 43 in the basal layer and connexin 26 from the basal to middle wing cell layers. In general, connexins are a family of proteins, commonly named according to their molecular weight or classified on a phylogenetic basis into alpha, beta, and gamma subclasses. Over 20 human isoforms have been identified. Different tissues and cell types are reported to have characteristic patterns of connexin protein expression and tissues have been shown to alter connexin protein expression pattern following injury or transplantation (Qui, C. et al., (2003) Current Biology, 13:1967-1703.

et alet alet al Polynucleotide-based therapeutic approaches, including antisense technology and RNA interference (RNAi), have been proposed for modulating expression of genes implicated in viral, fungal, and metabolic diseases. See, e.g., U.S. Pat. No. 5,166,195 (oligonucleotide inhibitors of HIV) and U.S. Pat. No. 5,004,810 (oligomers for hybridizing to herpes simplex virus Vmw65 mRNA and inhibiting replication). See also U.S. Pat. Nos. 7,098,190, 7,879,811, 7,902,164, and 7,919,474 (each entitled, “Formulations comprising antisense nucleotides to connexins”). Peptide inhibitors of gap junctions and hemichannels have also been reported. See, e.g., Berthoud, et al., Am J. Physiol. Lung Cell Mol. Physiol. 279:L619-L622 (2000); Evans and Boitano, Biochem. Soc. Trans. 29:606-612, and De Vriese, et al., Kidney Int. 61:177-185 (2001). See also PCT/US06/04131 (“Anti-connexin compounds and uses thereof”), US PG Pub. Nos. 20110217313 (“Treatment of Orthopedic Conditions”), 20110144182 (“Treatment of Surgical Adhesions”), 20110136890 (“Treatment of Fibrotic Conditions”), 20110130710 (“Treatment of Abnormal or Excessive Scars”), 20110065770 (“Formulations comprising antisense nucleotides to connexins”), 20090220450 (“Methods and compositions for wound healing”), and 20080249041, 20080221051, 20070078103, 20070072820, 20070072819, 20070066555, 20070060538, and 20070037765 (each entitled, “Formulations comprising antisense nucleotides to connexins”).

Despite advances in the understanding of the principles underlying the wound healing process, there remains a significant unmet need in suitable therapeutic options for wound care, including wounds that do not heal at expected rates, such as delayed-healing wounds, incompletely healing wounds, and chronic wounds. Such therapeutics compositions and treatments are described and claimed herein.

### BRIEF SUMMARY OF THE INVENTION

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this Brief Summary, which is included for purposes of illustration only and not restriction.

The invention generally relates to the use of an anti-cadherin agent, preferably an anti-cadherin polynucleotide species, alone or in combination with one or more other agents useful in the treatment of acute, delayed healing and chronic wounds.

Examples of such other agents include anti-connexin agents, for example anti-connexin polynucleotides (for example, connexin inhibitors such as alpha-1 connexin oligodeoxynucleotides), anti-connexin peptides (for example, antibodies and antibody binding fragments) and peptidomimetics (for example, alpha-1 anti-connexin peptides or peptidomimetics), gap junction closing or blocking compounds, hemichannel closing or blocking compounds, and connexin carboxy-terminal polypeptides, e.g., polypeptides that bind to ZO-1 or a ZO-1 binding site, anti-ZO-1 polynucleotides, as well as anti-ostepontin agents, particularly anti-osteopontin polynucleotides. Preferred combinations include an anti-N-cadherin polynucleotide species and an anti-connexin polynucleotide species (particularly an anti-connexin 43 polynucleotide species) and/or a polynucleotide species that targets ZO-1 expression.

Compositions and methods of the invention that employ one or more anti-cadherin agent species for the treatment of, for example, acute, delayed healing, and chronic wounds are described and claimed. Preferred compositions include therapeutically useful compositions, particularly pharmaceutical or veterinary compositions that comprise a therapeutically acceptable amount of one or more anti-cadherin agent species in amounts effective to promote healing or tissue repair in a subject. In preferred embodiments, such compositions comprise a therapeutically acceptable amount of one or more anti-cadherin agent species in amounts effective to downregulate or otherwise lessen the expression or presence of one or more cadherin species; for example, one or more cadherin species at and/or around an injury or wound site. As a result, healing of the injury or wound can be initiated and/or enhanced, and inflammation and/or scarring reduced.

Preferred anti-cadherin agents are anti-cadherin polynucleotides. In one embodiment the anti-cadherin polynucleotides are anti-N-cadherin oligodeoxynucleotides (ODN). Preferred peptides or peptidomimetics, are anti-cadherin peptides or peptidomimetics, e.g., cadherin complex blocking peptides (for example, anti-cadherin antibodies and antibody binding fragments) or peptidomimetics (for example, peptidometics directed against one or more regions of cadherin. Preferred cadherin complex blocking compounds are cadherin extracellular polypeptides. Peptidomimetics may be administered per se, or complexed to one or more other agents, for example, antennapedia in order to facilitate membrane transport.

The compositions of the invention include, for example, topical and inhaled delivery forms and formulations. Such delivery forms and formulations include those for the treatment of a subject, as described herein.

Pharmaceutical compositions are also provided in the form of a combined preparation, for example, as an admixture of one or more distinct anti-cadherin agent species, alone in conjunction with and one or more therapeutic agent species that are not anti-cadherin agents, for example, one or more anti-connexin or anti-osteopontin agents, including anti-connexin and anti-osteopontin polynucleotides, peptide, and or peptidomimetic species.

The term “a combined preparation” includes a “kit of parts” in the sense that the combination partners as defined herein can be dosed independently or by use of different fixed combinations with distinguished amounts of the two or more agent species, i.e. simultaneously, separately or sequentially. The parts of the kit can then, for example, be administered simultaneously or chronologically staggered, that is, at different time points, with equal or different time intervals, and/or in the same or different numbers of dosings for any part of the kit of parts.

In some embodiments, a combined preparation is administered, wherein two or more separate compositions are administered to a subject, wherein the first composition comprises a therapeutically effective amount of an anti-cadherin agent and the second composition comprises a therapeutically effective amount of an anti-connexin polynucleotide, peptide, or peptidomimetic. In other embodiments, a third composition is administered comprising one or more anti-osteopontin polynucleotides, peptides, or peptidomimetics.

Pharmaceutical compositions are provided for combined, simultaneous, separate sequential, or sustained administration. In some embodiments, a composition comprising one or more anti-cadherin agents is administered at or about the same time as one or more anti-connexin agents and/or anti-osteopontin agents. In one embodiment, a composition comprising one or more anti-cadherin agents is administered within at least about thirty, sixty, ninety, or one hundred twenty minutes, or about 3, 4, 5, 6, 8, 12, 24, 48, or 168 hours of one or more anti-connexin agents and/or anti-osteopontin agents.

In one aspect, the invention includes pharmaceutical compositions, including topical, systemic, and inhaled delivery forms and formulations, comprising a pharmaceutically acceptable carrier and therapeutically effective amounts of an anti-cadherin agent species, alone or in combination with a different anti-cadherin agent species and/or one or more other therapeutic agent species, e.g., a first anti-connexin agent species, a second anti-connexin agent species, a first anti-osteopontin agent species, and/or and a second anti-osteopontin agent species. Such compositions are useful, for example, for wound healing.

Examples of anti-cadherin agents are anti-cadherin polynucleotides, including the anti-cadherin antisense oligodeoxynucleotides (“ODN”) described below. Examples of anti-cadherin polynucleotides include anti-cadherin oligodeoxynucleotides, including antisense (including modified and unmodified backbone antisense), RNAi, and miRNA and siRNA. Suitable anti-cadherin peptides include peptides that bind cadherin extracellular domains, for example, or cadherin intracellular domains. Suitable anti-cadherin agents include, for example, antisense ODNs, peptides, and peptidomimetics against N-cadherin, E-cadherin, P-cadherin, cadherin 11, cadherin 12, a protocadherin protein, a desmoglein protein, and a desmocollin protein. Included peptides or peptidomimetics are anti-cadherin peptides or peptidomimetics, e.g., cadherin complex blocking peptides (for example, anti-cadherin antibodies and antibody binding fragments) or peptidomimetics (for example, peptidometics directed against one or more extracellular or intracellular regions of cadherin). Peptidomimetics may be complexed to one or more other agents, for example, antennapedia in order to facilitate membrane transport for binding to intracellular cadherin regions and domains.

The present invention provides for an increase in the rate, extent, and/or quality of wound healing through the use of at least one anti-cadherin agent species, alone or in combination with one or more therapeutic agent species administered simultaneously, separate, or sequentially.

The present invention provides for a decrease in inflammation through the use of at least one anti-cadherin agent species, alone or in combination with one or more therapeutic agent species administered simultaneously, separate, or sequentially.

The present invention provides for a decrease in scarring and/or an increased quality of scar through the use of at least one anti-cadherin agent species, alone or in combination with one or more therapeutic agent species administered simultaneously, separate, or sequentially.

In certain embodiments, the combined use of an anti-cadherin agent in combination with one or more other therapeutic agents, for example, one or more anti-connexin polynucleotides, peptides, or peptidomimetics and/or one or more anti-osteopontin polynucleotides, peptides, or peptidomimetics has an additive, synergistic, or super-additive effect in the promotion of the desired therapeutic outcome, for example, wound healing and for reduced inflammation and scarring. In some of these preferred embodiments, the administration of a combined preparation will have fewer administration time points and/or increased time intervals between administrations as a result of such combined use. In other such preferred embodiments, the combined use allows a reduced frequency of administration. In other preferred embodiments, combined use allows the use of reduced doses of such agents compared to the dose or doses that may be effective when the agent is administered alone.

In another aspect, the invention includes methods for administering a therapeutically effective amount of an anti-cadherin agent, alone or in combination with one or more therapeutic anti-connexin agents. In some embodiments, the compositions are formulated, for example, in a delayed release preparation, a slow release preparation, an extended release preparation, a controlled release preparation, and/or in a repeat action preparation suitable for administration to a subject having a wound, including chronic wounds and wounds characterized in whole or in part by slow, delayed, or incomplete wound healing. Chronic wounds include diabetic ulcers (e.g., diabetic foot ulcers), venous ulcers, venous stasis ulcers, pressure ulcers, decubitus ulcers, vasculitic ulcers, arterial ulcers, infectious ulcers, burn ulcers, trauma-induced ulcers, inflammatory ulcers, and ulcerations associated with pyoderma gangrenosum. Chronic wounds also include ocular ulcers, including persistent epithelial defects. In some embodiments, the subject is diabetic; in others, the subject has a cardiovascular disease or condition, for example, venous hypertension, venous insufficiency and/or arterial insufficiency.

In certain other aspects, the invention relates to methods of using the compounds and compositions of the invention to treat subjects suffering from or at risk for various diseases, disorders, and conditions associated with a wound, including acute wounds and wounds that do not heal at expected rates, including delayed healing and chronic wounds. Treatment of a subject, e.g., for a wound, with one or more pharmaceutical compositions of the invention, e.g. one or more anti-cadherin agents, may comprise their simultaneous, separate, sequential or sustained administration.

In yet another aspect, the invention includes methods for treating a subject having or suspected of having or predisposed to, or at risk for, any diseases, disorders and/or conditions characterized in whole or in part by a wound or a tissue in need of repair. Such compositions include, for example, topical and inhaled delivery forms and formulations.

In another aspect, the invention provides methods of treatment comprising administering to a subject a pharmaceutical composition of the invention for use in the treatment of a wound, including for example, acute, as well as wounds that do not heal at expected rates, including delayed healing and chronic wounds.

In another aspect, the invention provides methods of treatment comprising administering to a subject in need thereof a composition comprising therapeutically effective amounts of an anti-cadherin agent, alone or in combination with one or more anti-connexin and/or anti-osteopontin agents. Also within the scope of the invention is pretreatment prior to surgery. This will reduce local damage at points of incision, excision or revision, for example, and prime cells for healing.

In yet another aspect, the invention provides methods of treatment comprising administering to a subject in need thereof a first composition and at least one other therapeutic composition (e.g., a second composition, second and third compositions, etc.). In embodiments of this aspect, the “first” composition comprises a therapeutically effective amount of an anti-cadherin agent, although this is not meant to imply that such composition is administered before, more frequently, or via a different route than the other therapeutic composition(s). In other words, in some of these embodiments, the first composition is administered first, while in others, the second composition is administered first. In embodiments involving the administration of three different therapeutic compositions, such methods, for example, can comprise simultaneous administration of each of the compositions according to the same or different dosing or administration regimen.

In a further aspect, the invention provides methods for improving or reducing scar formation in a subject in need thereof, for improving or reducing fibrosis in a subject, and for improving or reducing adhesion formation in a subject, comprising administering to said subject a therapeutically effective amount of a pharmaceutical composition comprising an anti-cadherin agent, alone or in combination with one or more other therapeutic agents.

Preferred methods of combination therapy include the sequential or simultaneous administration one or more anti-cadherin agents alone or in combination with one or more other therapeutic agent species, either, some, or all of which are provided in amounts or doses that are less that those used when the agent or agents is/are administered alone, i.e., when they are not administered in combination, either physically or in the course of treatment of a wound or other condition to be improved. Such lesser amounts of agents administered are typically from about one-half, one-third, one-fourth, one-fifth, one-sixth, one-eighth, one-tenth, or about one-twentieth the amount when administered alone.

In a further aspect, the invention includes transdermal patches, dressings, pads, wraps, matrices, and bandages capable of being adhered or otherwise associated with the skin of a subject, said articles being capable of delivering a therapeutically effective amount of an anti-cadherin agent, alone or in combination with one or more therapeutic agents, to a subject.

In another aspect, the invention includes an article of manufacture comprising a vessel containing a therapeutically effective amount of one or more anti-cadherin agents, alone or in combination with one or more other therapeutic agents, and instructions for use, including use for the treatment of a subject.

The invention includes an article of manufacture comprising packaging material containing one or more dosage forms containing one or more anti-cadherin agents, alone or together with dosage forms containing one or more other therapeutic agents, wherein the packaging material has a label that indicates that the dosage form can be used for a subject having or suspected of having or predisposed to any of the diseases, disorders and/or conditions described or referenced herein, including diseases, disorders and/or conditions characterized in whole or in part by acute, impaired, delayed or chronic wound healing, by scarring, by fibrosis, or by adhesions. Such dosage forms include, for example, topical delivery forms and formulations, powdered delivery forms and formulations, delivery forms and formulations suitable for injection or infusion (including dry or powdered compositions that must be reconstituted with a suitable diluent prior to administration), and delivery forms and formulations suitable for instillation. Suitable formulations deliver an amount of a therapeutic agent suitable to achieve a desired therapeutic effect. Preferred topical formulations include foams, sprays, and gels. Preferred gels are polyoxyethylene-polyoxypropylene copolymer-based gels and carboxymethylcellulose-based and related cellulose gels, with pluronic gels being particularly preferred.

The invention also includes methods for the use of therapeutically effective amounts of compositions of the invention in the manufacture of medicaments, including, for example, topical delivery forms and formulations. Such medicaments include those for the treatment of a subject as described herein.

In another aspect, the invention provides for the use of one or more anti-cadherin agents in the manufacture of pharmaceutical products for the promotion of wound healing, improved and/or reduced scarring, improved and/or reduced inflammation, reduced fibrosis, or reduced adhesion formation in a patient in need thereof. In some of these embodiments, the product includes a wound dressing or wound healing promoting matrix. Preferably, the wound dressing or matrix is provided in the form of a solid substrate with a composition comprising an anti-cadherin agent dispersed on or in the solid substrate.

In yet another embodiment, the invention provides for the use of compounds and compositions of the invention in conjunction with connective tissue growth factor (CTGF) inhibitors, e.g., CTGF antisense compounds. In another embodiment, the invention provides for the use of compounds and compositions of the invention in conjunction with PDGF receptor inhibitors to, for example, treat wounds and/or to reduce adhesions and scar formation. PDGF receptor inhibitors include, for example, receptor blockers, receptor antagonists. CTGG and PDGF receptor inhibitors also include monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies and antibody fragments which are capable of binding an antigenic determinant (e.g., an epitope) that makes contact with a particular antibody or other binding molecule, including antibodies and antibody binding fragments directed against CTGF or PDGF receptors.

In yet another embodiment, the invention provides for the use of compounds and compositions of the invention in conjunction with the application of artificial skin products, including, for example Dermagraft® (a single-layered cryopreserved dermal substitute composed of human fibroblasts, extracellular surrounding substance and a bioabsorbable framework), Apligraf® (living, bilayered skin construct with an epidermal layer formed by human keratinocytes and a dermal layer composed of human fibroblasts in a bovine Type 1 collagen web), Integra® (two-layer membrane system for skin replacement comprising a dermal replacement layer made of a porous template of fibers of bovine tendon collagen and glycosaminoglycan (chondroitin-6-sulfate) and an epidermal substitute layer made of thin silicone to control moisture loss), AlloDerm® (acellular dermal matrix), Cyzact™ (human dermal fibroblasts delivered via a fibrin), ICX-SKN (a combination of fibroblasts and fibrin matrix that are remodeled to produce a collagen matrix), Keragraft™ (a human stem cell-derived product being developed for wound care as an autologous epidermal equivalent), OASIS® Wound Matrix (biologically derived extracellular matrix-based wound product created from porcine-derived acellular small intestine submucosal), OrCel™ (two-layer cellular template in which human epidermal keratinocytes and dermal fibroblasts are cultured in two separate layers onto a bovine collagen sponge), TransCyte® (human fibroblast-derived temporary skin substitute consisting of a polymer membrane and neonatal human fibroblast cells), and so on. The compounds and compositions of the invention are also useful in conjunction with the application of other dressings to promote wound healing, including, for example, BioBrane. The compounds and compositions of the invention may also be used in conjunction with the application of other types of scaffolds or dressings to promote wound healing, including, for example, spray on cells being developed by HealthPoint (a cell therapy spray suspension known as HP802-247, which consists of two components that are sprayed sequentially on the wound bed at the time of treatment: a fibrinogen solution and a cell preparation containing a mixture of growth arrested, living, allogeneic epidermal keratinocytes and dermal fibroblasts) and cultured allogenic keratinocytes.

The inventions also relate to the use of an anti-ZO-1 agent, including peptides and peptidopreferably an anti-ZO-1 polynucleotide species, alone or in combination with one or more other agents useful in the treatment of acute, delayed healing and chronic wounds.

In another aspect, the inventions relate to the use of (a) anti-connexin agents, preferably anti-connexin43 agents, and/or (b) anti-cadherin agents, preferably anti-N-cadherin agents, most preferably anti-connexin43 and/or anti-N-cadherin polynucleotides (including, for example, antisense polynucleotides) to increase Rac1 and RhoA GTPase activity in vivo and in vitro.

In another aspect, the inventions relate to the use of (a) anti-connexin agents, preferably anti-connexin43 agents, and/or (b) anti-cadherin agents, preferably anti-N-cadherin agents, most preferably anti-connexin43 and/or anti-N-cadherin polynucleotides (including, for example, antisense polynucleotides) to induce cytoskeletal changes and increase lamellipodial protrusions in cells in vitro and in vivo.

In another aspect, the inventions relate to the use of (a) anti-connexin agents, preferably anti-connexin43 agents, and/or (b) anti-cadherin agents, preferably anti-N-cadherin agents, most preferably anti-connexin43 and/or anti-N-cadherin polynucleotides (including, for example, antisense polynucleotides) to reduce cellular adhesion.

In another aspect, the inventions relate to the use of an agent to reduce calcium, for example, agents to reduce extracellular calcium for uses in reduction of cadherin activity, for example, N-cadherin activity, alone or in conjunction with other anti-cadherin agents.

These and other aspects of the present inventions, which are not limited to or by the information in this Brief Summary, are provided below.

### BRIEF DESCRIPTION OF THE FIGURES

The patent or application file contains at least one drawing executed in color. Copies of this patent or patent application publication with color drawings will be provided by the Office upon request and payment of the necessary fee.

This application contains at least one figure executed in color. Copies of this application with color figures will be provided upon request and payment of the necessary fee. A brief summary of each of the figures is provided below.

FIG. 1. Dermal Cx43 is greatly upregulated in human chronic VLU. (A) Cx43 expression levels are reduced at the dermal wound margins 4 hours after leg punch biopsy of the skin of healthy volunteers. Scale bar=25 μm. Blue signal is Hoechst staining of nuclei and collagen bundle autofluorescence. The dotted white line shows the border between the epidermis and the dermis. (B) Cx43 levels increased with progressive distance from the injury site (C). Values represent mean±SD (n=3; **p<0.01). (D) A representative picture of a chronic VLU in the lower leg of a patient, from which a wound edge punch biopsy has been taken—white dotted circle. (E) Dermal Cx43 expression levels were significantly upregulated in chronic VLU in comparison to matched non-wounded samples (n=6; *** p<0.005). (F) Graph depicting Cx43 levels in VLU vs. non-wounded skin. Values were expressed as mean±SEM. Scale bar=25 μm.

FIG. 2. Increased expression of N-cadherin and ZO-1 in the dermis of human chronic VLU (A) ZO-1 expression levels are elevated in the dermis of chronic VLU compared to matched non-wounded controls (n=6). Scale bar=25 μm. Higher magnifications of VLU and intact skin (boxed regions 1 and 2) stained for ZO-1 (green) and Hoechst (blue) are shown. Scale bar=10 μm. (B) N-cadherin is significantly upregulated in chronic VLU compared to matched non-wounded samples (n=6). Scale bar=25 μm. The boxed regions 1 and 2 show high magnifications of VLU and non-wounded skin samples stained for N-cadherin (green) and Hoechst (blue). Scale bar=10 μm. Values represent mean±SD. (C and D) Graphs show ZO-1 and N-cadherin expression levels in VLU vs. non-wounded skin. Values for ZO-1 and N-cadherin were expressed as mean±SD; **p<0.01 and p<0.005, respectively.

FIG. 3. Targeting Cx43 reduces N-cadherin and ZO-1 expression in fibroblasts. (A) ZO-1 or (B) N-cadherin expression levels were examined in mouse skin wounds treated in vivo with Cx43sODN or Cx43asODN (n=6). Downregulation of ZO-1 and N-cadherin was found in the dermis of mice after Cx43 knockdown. Graphs represent mean±SD; * p<0.05 and **p<0.01. (C-D) ZO-1 and Cx43 levels were analyzed by Western blot in Cx43shRNA and p.Sup-transduced fibroblasts. Tubulin was included as loading control. Quantification shows that knockdown of Cx43 reduced the expression of ZO-1 (***p<0.005; n=3). (E) ZO-1 (red) and Cx43 (green) expression and distribution in leading edge (LE) and internal areas (IA) were analyzed by confocal microscopy in wounded Cx43shRNA or p.Sup-transduced cells (n=4). Cells were also counterstained with Hoechst (blue). Scale bar=20 μm. (F) N-cadherin expression levels were significantly reduced after targeting Cx43 (**p<0.01; n=3). (G) N-cadherin (green) redistribution from membrane (arrowheads) to cytosolic compartments (arrows) was analyzed along with F-Actin (red) in p.Sup and Cx43shRNA fibroblasts 3 h after scratch wounding of confluent 3T3 monolayers (n=3). Scale bar=20 μm.

FIG. 4. Cx43 and N-cadherin knockdown accelerates the rate of fibroblast migration. (A and B) N-cadherin and ZO-1 expression was evaluated by Western blot in Mock or N-cadshRNA-transduced cells, and in ZO-1 asODN or sense-treated (ZO-1sODN) cells, respectively. Values represent mean±SD (n=3, ***p<0.005; n=3, *p<0.05). (C) Cx43shRNA and p.Sup cells, Mock and N-cadshRNA cells, or fibroblasts treated with ZO-1sODN and ZO-1 asODN were allowed to migrate into a wound for 3 h. Pictures of cells at the beginning of the migration recording (0 h) and at the end (3 h) are shown. (D) The graph shows the velocity of migration for all the aforementioned conditions. Values represent mean±SEM (***p<0.005).

FIG. 5. Cx43 and N-cadherin contribute to cell polarization, adhesion, and proliferation in fibroblasts. (A-B) Cell-cell adhesion upon suspension in hanging drops was assessed for Cx43shRNA and p.Sup, and for Mock and N-cadshRNA cells. The areas of cell clusters in six random fields taken from six different hanging drops were determined for each condition. Graphs represent the average number of clusters falling into each of three size ranges for each condition. Scale bar=300 μm. (C-D) Cx43shRNA and p.Sup, and Mock and N-cadshRNA cells were wounded, allowed to migrate for 3 h, and then fixed and immunostained with anti-GM130 (red) and Hoechst (blue). Scale bar=25 μm. The percentage of cells with Golgi located in the 120° arc facing the wound were scored as positive. Between 66-106 cells were evaluated in n=3 independent experiments. Data represent the mean±SEM (*P<0.05). Growth curves showing cell proliferation of fibroblasts transduced with (E) p.Sup and Cx43shRNA, and (F) Mock and N-cadshRNA constructs are shown. Data represent mean±SD of three independent experiments, performed in duplicate.

FIG. 6. Targeting Cx43 induces cytoskeletal changes in leading-edge fibroblasts. (A) Representative images showing the distribution of F-actin (red), and tyrosinated (green) and acetylated (blue) tubulin (TyrTub and AcetTub, respectively) in leading edge Cx43sODN and C43asODN fibroblasts, 3 h after wounding. Cells were also counterstained with the nuclear marker Hoechst. Scale bar=20 μm. (B) The graph shows the length of the protrusions of wound edge cells. Data represent the distance (mean±SEM) from the nucleus to the leading edge in n=3 experiments (***P<0.005).

FIG. 7. Modulation of Cx43 levels influence wound-edge cytoskeletal architecture in fibroblasts. Representative images of leading-edge cells transfected (arrowhead) with (A) a control pGFP construct (B) Cx43-DN, or (C) Cx43-WT, stained for TyrTub, F-actin and Hoechst. Scale bar=20 μm. (D) Graph depicting the length of lamelipodial protrusions of fibroblasts transfected with the different constructs is shown (**p<0.01; ***P<0.005; n=3).

FIG. 8. Cx43 and N-cadherin knockdown increases Rac1 and RhoA GTPase activity in fibroblasts. (A) Rho GTPase (GTP) activities in wounded fibroblasts were measured by pull-down assays using the PBD domain of PAK (Rac1 and Cdc42), or the RBD domain of Rhotekin (RhoA), followed by immunoblotting with the respective antibodies. Additionally, Rac1, Cdc42 and RhoA from total lysates were used as loading controls. (B) Graphs show active Rac1 and RhoA GTPase activity (GTP levels/total levels; mean±SEM, *P<0.05); n=3 independent experiments. Representative images of leading edge Cx43shRNA and p.Sup transduced cells showing (C) Rac1, and (D) RhoA GTPase activities, 3 h after wounding of confluent monolayers. Scale bar=10 μm. Arrows indicate the direction of migration. (E) FRET efficiency analysis show a two-fold increase in Rac1 and RhoA activities in Cx43shRNA vs. p.Sup-transduced cells, while no differences were observed for Cdc42. Data is representative of n=3 experiments per condition; *p<0.05.

FIG. 9 (S1). Cx43 is downregulated in fibroblasts after wounding. (A) The expression and distribution of Cx43 in mouse's skin dermis was examined by immunohistochemistry 2 d after excisional wounding. Wound-edge Cx43 was reduced in dermal fibroblasts after such wounds. Arrowheads show how Cx43 becomes more prevalent with increasing distance from the wound edge. Scale bar=25 μm. Cx43 levels were quantified along the wound site and were significantly lower at the wound edge; p<0.005. Values are expressed as mean±SD. (B) ZO-1 (green) and Cx43 (red) were examined in mouse skin wounds treated in vivo with Cx43sODN or Cx43asODN (n=6). Cells were also counterstained with Hoechst (blue). A clear downregulation of ZO-1 was found in the dermis of mice treated with the Cx43asODN. Scale bar=100 μm. (C) Excisional wounds treated with Cx43asODN or control Cx43sODN were used to evaluate the distribution of N-cadherin (green) by immunohistochemistry. Scale bar=100 μm.

FIG. 10 (S2). Knockdown of Cx43 induces cytoskeletal changes. (A) Cx43 expression levels and (B) cell-cell communication were evaluated in Cx43asODN or LiCl-treated 3T3 fibroblasts, and in untreated or sense-treated (Cx43-sODN) controls. Values are expressed as mean±SD (*p<0.05; **p<0.01 and ***p<0.005; n=4). (C) Confluent monolayers treated with Cx43sODN, Cx43asODN or LiCl were wounded and allowed to migrate for 3 hours. Pictures of cells at the beginning of the migration recording (0 hours) and at the end (3 hours) are shown. Scale bar=25 μm. (D) The graph shows the velocity of migration, which was inversely correlated with both dye coupling and Cx43 expression. Values represent mean±SEM (*p<0.01; ***p<0.05; n=6). (E) Cells transfected with Cx43-DN, Cx43-WT or pGFP constructs (n=3), as well as Cx43shRNA or p.Sup-infected cells (n=6), were allowed to migrate into a wound for 3 hours. Images were taken at the beginning of the migration recording (0 hours; arrows pointing to Cx43-DN, Cx43-WT, or pGFP-transfected cells) and 3 hours later (arrowheads pointing to Cx43-DN, Cx43-WT or pGFP). Cx43shRNA and Cx43-DN cells accelerated migration, while Cx43-WT slowed it. Scale bar=25 μm. (F) The graph shows the velocity of migration for each treatment and confirms that either silencing of Cx43 or transfection with Cx43-DN induce a significant increase in the rate of migration relative to the other conditions analyzed. Data are expressed as mean±SD (*p<0.05; **p<0.01; n=4). (G) Cx43 levels and dye coupling after LY microinjection were assessed in Cx43shRNA or p.Sup-infected 3T3 fibroblasts. Cx43shRNA was effective in downregulating Cx43 and eliminating communication with neighbouring cells. Values represent mean±SEM for Cx43 levels, and mean±SD for cell coupling (***p<0.005; n=4). (J) Cx43shRNA and p.Sup cells were grown in the presence (FBS) or absence (SS) of serum and allowed to migrate into a wound for 4 h. The graph shows the velocity of migration; values represent mean±SEM of n=3 independent experiments (***p<0.005).

FIG. 11 (S3). Analysis of α- and β-catenin expression and distribution after targeting Cx43. (A and C) Protein distribution of α- and β-catenin was analyzed in p.Sup and Cx43shRNA-infected fibroblasts 3 h after wound scratch of confluent 3T3 monolayers. Arrows in (A) indicate cytoplasmic relocation of β-catenin from the plasma membrane to the cytosol in Cx43shRNA-infected cells. The single arrowhead indicates a site of putative nuclear localization of β-catenin in Cx43shRNA-infected fibroblasts. Scale bar=25 μm. (B and D). The expression levels of these proteins were also evaluated by Western blot and values were normalized with respect to actin or tubulin (Tub).

FIG. 12 (S4). Knockdown of Cx43 induces cytoskeletal changes. The distribution of TyrTub, AcetTub, and F-actin was studied 3 h after wounding confluent monolayers of Cx43shRNA and p.Sup transduced fibroblasts. Scale bar=25 μm. The graph shows the length of the protrusions of wound edge cells and data represent the distance from the nucleus to the leading edge (mean±SEM; n=3 experiments; ***P<0.005).

FIG. 13 (S5). Knockdown of N-cadherin increases lamellipodial protrusions. (A) The distribution of F-actin was studied 3 h after wounding confluent monolayers of Cx43shRNA and p.Sup transduced fibroblasts. Targeting N-cadherin induces cytoskeletal changes in wound edge cells. Scale bar=25 μm. (B) The graph shows the length of the protrusions of control (Mock) and NcadshRNA wound edge cells. Data represent the distance from the nucleus to the leading edge (mean±SEM; n=3 experiments; ***P<0.005).

### DETAILED DESCRIPTION OF THE INVENTION

### Definitions

As used herein, a “disorder” is any disorder, disease, or condition that would benefit from an agent that initiates, accelerates, promotes or enhances wound healing (including acute wounds, dehiscent wounds, and slow-healing delayed-healing and chronic wounds), reduces inflammation, reduces or lessens scarring, improves scar quality), reduces fibrosis, and/or reduces adhesions. For example, diseases, disorders, and conditions include acute wounds. Diseases, disorders, and conditions also include dehiscent wounds, and slow-healing delayed-healing and chronic wounds. Also included are diseases, disorders, and conditions characterized by excess production of fibrous material, including excess production of fibrous material within the extracellular matrix. Also included are diseases, disorders and conditions characterized by replacement of normal tissue elements by abnormal, non-functional, and/or excessive accumulation of matrix-associated components. Also included are diseases, disorders and conditions characterized by adhesion formation. Also included is any disorder, disease, or condition that would benefit from an agent that promotes wound healing and/or reduces swelling, inflammation, and/or scar formation (including abnormal and excessive scarring, including keloid scars, hypertrophic scars, widespread (stretched) scars, and atrophic (depressed) scars). For example, included are wounds resulting from surgery or trauma, wounds that do not heal at expected rates (such as delayed-healing wounds, incompletely healing wounds, chronic wounds, and dehiscent wounds), and wound associated abnormalities in connection with neuropathic, ischemic, microvascular pathology, pressure over bony area (tailbone (sacral), hip (trochanteric), buttocks (ischial), or heel of the foot), reperfusion injury, and valve reflux etiology and conditions. Also included are diseases, disorders and conditions characterized by unwanted ZO-1 protein or ZO-1 protein activity or that would benefit from reduced ZO-1 protein or ZO-1 protein activity. Also included are diseases, disorders and conditions characterized by unwanted reduced Rac1 or Rac1 activity or that would benefit from increased Rac1 or Rac1 activity. Also included are diseases, disorders and conditions characterized by unwanted reduced RhoA GTPase or RhoA GTPase activity or that would benefit from increased RhoA GTPase or RhoA GTPase activity. Also included are diseases, disorders and conditions characterized that would benefit from enhanced cellular migration, lessened cellular adhesion, cellular cytoskeletal changes as described herein, and/or increased cellular lamellipodial protrusions as described herein.

As used herein, “subject” refers to any mammal, including humans, domestic and farm animals, and zoo, sports, and pet animals, such as dogs, horses, cats, sheep, pigs, cows, etc. The preferred mammal herein is a human, including adults, children, and the elderly. A subject may also be a bird, including zoo, sports, and pet birds. Preferred sports animals are horses and dogs. Preferred pet animals are dogs and cats.

As used herein, “preventing” means preventing in whole or in part, or ameliorating or controlling, or reducing or halting the production or occurrence of the thing or event to be prevented.

As used herein, a “therapeutically effective amount” or “effective amount” in reference to the compounds or compositions of the instant invention refers to the amount sufficient to induce a desired biological, pharmaceutical, or therapeutic result. That result can be alleviation of the signs, symptoms, or causes of a disease or disorder or condition, or any other desired alteration of a biological system. In the present invention, the result will involve preventing fibrosis. In another aspect of the present invention, the result will involve the prevention and/or reduction of adhesions. In another aspect of the present invention, the result will involve the prevention and/or reduction of scarring and abnormal scarring, as well as prevention and/or reduction of excessive scar formation and other types of abnormal proliferation of tissue, including keloid scars, hypertrophic scars, widespread scars, and atrophic scars.

According to a further aspect, the result will involve the promotion and/or improvement of wound healing and closure of wounds, in whole or in part, including improvements in rates of healing. Other benefits include reductions in swelling, inflammation and/or scar formation, in whole or in part. Still other benefits reduced ZO-1 protein or ZO-1 protein activity, increased Rac1 or Rac1 activity, increased RhoA GTPase or RhoA GTPase activity or that would benefit from increased RhoA GTPase or RhoA GTPase activity. Also included are diseases, disorders and conditions characterized by unwanted reduced RhoA GTPase or RhoA GTPase activity. Still other benefits are cellular cytoskeletal changes as described herein, and increased cellular lamellipodial protrusions as described herein.

As used herein, the terms “treating” and “treatment” refer to both therapeutic treatment and prophylactic or preventative measures. Those in need of treatment include those already with the disorder as well as those prone to having the disorder or diagnosed with the disorder or those in which the disorder is to be prevented. Thus, by way of example, the promotion of wound healing, the reduction of inflammation, the promotion of cell migration, the reduction of cellular adhesion, anti-fibrotic applications of compounds and compositions and formulations of the invention administered prior to the formation of fibrosis or fibrotic tissue are within the invention, as are anti-adhesion applications of compounds and compositions and formulations of the invention administered prior to the formation of an adhesion, and anti-scarring applications of compounds and compositions and formulations of the invention administered prior to scar formation including, for example, in a scar reduction surgery or procedure.

As used herein, “anti-cadherin agents” are compounds that affect or modulate the activity, expression, or formation of a cadherin protein. Anti-cadherin agents include, without limitation, anti-cadherin polynucleotides, which include antisense compounds (e.g., antisense polynucleotides), RNAi, miRNA and siRNA compounds; antibodies and antigen-binding fragments thereof; and peptides and polypeptides, which include “peptidomimetics” and peptide analogs. In addition to anti-cadherin polynucleotides and anti-cadherin peptides, peptidomimetics, or adherens junction modifying agents, other anti-cadherin agents include adherens junction disrupting compounds (e.g., calcium-ion binding compounds), and cadherin carboxy-terminal polypeptides (which can, e.g., block or disrupt cadherin-cadherin protein interactions between adjoining cells, thereby disrupting adherens junction formation and/or maintenance). Preferred anti-cadherin agents are anti-N-cadherin agents. Exemplary anti-cadherin agents are discussed in further detail herein.

The terms “peptidomimetic” and “mimetic” include naturally occurring and synthetic chemical compounds that may have substantially the same structural and functional characteristics of protein regions that they mimic. In the case of cadherin proteins, these may mimic, for example, the extracellular loops of cadherin-repeating domains in the extracellular region of cadherin proteins involved in cadherin repeat association, adherens junction formation and maintenance, and cell-cell adhesion.

“Peptide analogs” refer to the compounds with properties analogous to those of the template peptide and may be non-peptide drugs. “Peptidomimetics” (also known as “mimetic peptides”), which include peptide-based compounds, also include such non-peptide based compounds such as peptide analogs. Peptidomimetics that are structurally similar to therapeutically useful peptides may be used to produce an equivalent or enhanced therapeutic or prophylactic effect. Generally, peptidomimetics are structurally identical or similar to a paradigm polypeptide (i.e., a polypeptide that has a biological or pharmacological function or activity), but can also have one or more peptide linkages optionally replaced by a linkage selected from the group consisting of, for example, —CH₂NH—, —CH₂S—, —CH₂—CH₂—, —CH═CH— (cis and trans), —COCH₂—, —CH(OH)CH₂—, and —CH₂SO—. The mimetic can be either entirely composed of natural amino acids, or non-natural analogues of amino acids, or, is a chimeric molecule of partly natural peptide amino acids and partly non-natural analogs of amino acids. The mimetic can also comprise any amount of natural amino acid conservative substitutions as long as such substitutions also do not substantially alter mimetic activity. For example, a mimetic composition may be useful as an anti-cadherin agent if it is capable of down-regulating biological actions or activities of cadherin proteins, cadherin complexes, or adherens junctions, such as, for example, preventing the head-to-head association of cadherin repeats of opposing cadherin extracellular domains on adjoining cells, association of cadherin proteins in the same cell, formation of cadherin complexes in cells, association of cadherin complexes with the actin cytoskeleton, and/or adherens junction formation. Peptidomimetics, mimetic peptides, and cadherin-modulating peptides, as well as compounds, including cadherin extracellular domains that comprise one or more cadherin repeats, encompass those described or referenced herein, as well as those as may be known in the art, whether now known or later developed.

The terms “modulator” and “modulation” of cadherin activity, as used herein in its various forms, refers to inhibition in whole or in part of the expression or action or activity of a cadherin protein, cadherin complex, or adherens junction, and may function as anti-cadherin agents.

In general, the term “protein” refers to any polymer of two or more individual amino acids (whether or not naturally occurring) linked via peptide bonds, as occur when the carboxylcarbon atom of the carboxylic acid group bonded to the alpha-carbon of one amino acid (or amino acid residue) becomes covalently bound to the amino nitrogen atom of the amino group bonded to the alpha-carbon of an adjacent amino acid. These peptide bond linkages, and the atoms comprising them (i.e., alpha-carbon atoms, carboxylcarbon atoms (and their substituent oxygen atoms), and amino nitrogen atoms (and their substituent hydrogen atoms)) form the “polypeptide backbone” of the protein. In addition, as used herein, the term “protein” is understood to include the terms “polypeptide” and “peptide” (which, at times, may be used interchangeably herein). Similarly, protein fragments, analogs, derivatives, and variants are may be referred to herein as “proteins,” and shall be deemed to be a “protein” unless otherwise indicated. The term “fragment” of a protein refers to a polypeptide comprising fewer than all of the amino acid residues of the protein. A “domain” of a protein is also a fragment, and comprises the amino acid residues of the protein often required to confer activity or function.

As used herein, “simultaneously” is used to mean that the one or more agents of the invention are administered concurrently, whereas the term “in combination” is used to mean they are administered, if not simultaneously or in physical combination, then “sequentially” within a timeframe that they both are available to act therapeutically. Thus, administration “sequentially” may permit one agent to be administered within minutes (for example, 1, 2, 3, 4, 5, 10, 15, 20, 25, 30) minutes or a matter of hours, days, weeks, or months after the other, provided that both are present in effective amounts. The time delay between administration or administrations of the components will vary depending on the exact nature of the components, the interaction between them, and their respective half-lives.

The term “dressing” refers to a dressing for topical application to a wound and excludes compositions suitable for systemic administration. For example, the one or more anti-cadherin agents may be dispersed in or on a solid sheet of wound contacting material such as a woven or nonwoven textile material, or may be dispersed in a layer of foam such as polyurethane foam, or in a hydrogel such as a polyurethane hydrogel, a polyacrylate hydrogel, gelatin, carboxymethyl cellulose, pectin, alginate, and/or hyaluronic acid hydrogel, for example in a gel or ointment. In certain embodiments the one or more anti-cadherin polynucleotides are dispersed in or on a biodegradable sheet material that provides sustained release of the active ingredients into the wound, for example a sheet of freeze-dried collagen, freeze-dried collagen/alginate mixtures (available under the Registered Trade Mark FIBRACOL from Johnson & Johnson Medical Limited) or freeze-dried collagen/oxidized regenerated cellulose (available under the Registered Trade Mark PROMOGRAN from Johnson & Johnson Medical Limited).

As used herein, “matrix” includes for example, matrices such as collagen, acellular matrices, crosslinked biological scaffold molecules, tissue-based matrices (including pig-based wound healing matrices), cultured epidermal autografts, cultured epidermal allografts, tissue-engineered skin, collagen and glycosaminoglycan dermal matrices inoculated with autologous fibroblasts and keratinocytes, AlloDerm® (a nonliving allogeneic acellular dermal matrix with intact basement membrane complex), living skin equivalents (e.g., Dermagraft® (living allogeneic dermal fibroblasts grown on degradable scaffold), TransCyte® (an extracellular matrix generated by allogeneic human dermal fibroblasts), Apligraf® (a living allogeneic bilayered construct containing keratinocytes, fibroblasts and bovine type I collagen), Integra® (two-layer membrane system for skin replacement comprising a dermal replacement layer made of a porous template of fibers of bovine tendon collagen and glycosaminoglycan (chondroitin-6-sulfate) and an epidermal substitute layer made of thin silicone to control moisture loss), Cyzact™ (human dermal fibroblasts delivered via a fibrin), ICX-SKN (a combination of fibroblasts and fibrin matrix that are remodeled to produce a collagen matrix), Keragraft™ (a human stem cell-derived product being developed for wound care as an autologous epidermal equivalent), OASIS® Wound Matrix (biologically derived extracellular matrix-based wound product created from porcine-derived acellular small intestine submucosal), and OrCel™ (allogeneic fibroblasts and keratinocytes seeded in opposite sides of bilayered matrix of bovine collagen), BioBrane, cultured allogenic keratinocytes, animal derived dressings (e.g., Oasis's porcine small intestinal submucosa acellular collagen matrix; and E-Z Derm's acellular xenogeneic collagen matrix), tissue-based bioengineered structural frameworks, scaffolds, biomanufactured bioprostheses, and other implanted or applied structures such as for example, vascular grafts suitable for cell infiltration and proliferation useful in the promotion of wound healing. A matrix is also provided by a cell therapy spray suspension known as HP802-247, being developed by HealthPoint, which consists of two components that are sprayed sequentially on the wound bed at the time of treatment: a fibrinogen solution and a cell preparation containing a mixture of growth arrested, living, allogeneic epidermal keratinocytes and dermal fibroblasts.

Additional suitable biomatrix material may include chemically modified collagenous tissue to reduce antigenicity and immunogenicity. Other suitable examples include collagen sheets for wound dressings, antigen-free or antigen reduced acellular matrix (Wilson, et al., Trans Am Soc Artif Intern 1990; 36:340-343), or other biomatrices that have been engineered to reduce the antigenic response to the xenograft material. Other matrices useful in promotion of wound healing may include for example, processed bovine pericardium proteins comprising insoluble collagen and elastin (Courtman, et al., J Biomed Mater Res 1994; 28:655-666) and other acellular tissue which may be useful for providing a natural microenvironment for host cell migration to accelerate tissue regeneration (Malone, et al., J Vasc Surg 1984; 1:181-91). In certain embodiments, the matrix material may be supplemented with one or more anti-cadherin agents, anti-ZO-1 agents, anti-connexin43 agents, and/or the one or more therapeutic agents for site-specific release of such agents.

### Wounds and Wound Classification

Chronic wounds, slow healing wounds, and incomplete healing wounds often result in infection and can lead to amputation or death. It has been discovered that use of certain compounds, including those described or referenced herein, may block, inhibit, or alter cell communications, which may promote closure and healing in chronic, slow healing, and incomplete healing wounds.

By “wound” is meant an injury to any tissue, including, for example, acute, delayed, slow, or difficult to heal wounds, and chronic wounds. Examples of wounds may include both open and closed wounds. Wounds include, for example, burns, incisions, excisions, lacerations, abrasions, puncture or penetrating wounds, surgical wounds, contusions, hematomas, crushing injuries, and ulcers. Also included are wounds that do not heal at expected rates.

By a “wound that does not heal at the/an expected rate” is meant an injury to any tissue that does not heal in an expected or typical time frame, including delayed, slow, or difficult to heal wounds (including delayed or incompletely healing wounds), and chronic wounds. Examples of wounds that do not heal at the expected rate include diabetic ulcers, diabetic foot ulcers, vasculitic ulcers, arterial ulcers, venous ulcers, venous stasis ulcers, pressure ulcers, decubitus ulcers, infectious ulcers, trauma-induced ulcers, burn ulcers, ulcerations associated with pyoderma gangrenosum, and mixed ulcers.

As described herein, a delayed or difficult to heal wound may include, for example, a wound that is characterized at least in part by one or more of 1) a prolonged inflammatory phase, 2) a slow forming extracellular matrix, and 3) a stalled or decreased rate of epithelialization.

In the art, the term “chronic wound” refers generally to a wound that has not healed within about three months, but can be wounds that have not healed within about one or two months. Chronic skin wounds include, for example, pressure ulcers, diabetic ulcers, venous ulcers, arterial ulcers, inflammatory ulcers, and mixed ulcers. The chronic wound may be an arterial ulcer that can include ulcerations resulting from complete or partial arterial blockage. The chronic wound may be a venous stasis ulcer, which can include ulcerations resulting from a malfunction of the venous valve and the associated vascular disease. The chronic wound may be a trauma-induced ulcer.

As used herein, chronic wound can also include, for example, a wound that is characterized at least in part by 1) a chronic self-perpetuating state of wound inflammation, 2) a deficient and defective wound extracellular matrix (ECM), 3) poorly responding (senescent) wound cells (e.g. fibroblasts), limited ECM production, and 4) failure of re-epithelialization due in part to lack of the necessary ECM orchestration and lack of scaffold for migration.

Chronic wounds can also be characterized, for example, by 1) prolonged inflammation and proteolytic activity, leading to ulcerative lesions, including, for example, diabetic, pressure (decubitus), venous, and arterial ulcers, 2) prolonged fibrosis in the wound leading to scarring, 3) progressive deposition of matrix in the affected area, 4) longer repair times, 5) less wound contraction, 6) slower re-epithelialization, and 7) increased thickness of granulation tissue.

Exemplary chronic wounds also include “pressure ulcers.” Exemplary pressure ulcers may include all four stages of wound classifications based on AHCPR (Agency for Health Care Policy and Research, U.S. Department of Health and Human Services) guidelines, including for example, Stage 1. A stage I pressure ulcer is an observable pressure related alteration of intact skin whose indicators as compared to the adjacent or opposite area on the body may include changes in one or more of the following: skin temperature (warmth or coolness), tissue consistency (firm or boggy feel), and/or sensation (pain, itching). The ulcer appears as a defined area of persistent redness in lightly pigmented skin, whereas in darker skin tones, the ulcer may appear with persistent red, blue, or purple hues. Stage 1 ulceration may include nonblanchable erythema of intact skin and the heralding lesion of skin ulceration. In individuals with darker skin, discoloration of the skin, warmth, edema, induration, or hardness may also be indicators of stage 1 ulceration. Stage 2: stage 2 ulceration may be characterized by partial thickness skin loss involving epidermis, dermis, or both. The ulcer is superficial and presents clinically as an abrasion, blister, or shallow crater. Stage 3: stage 3 ulceration may be characterized by full thickness skin loss involving damage to or necrosis of subcutaneous tissue that may extend down to, but not through, underlying fascia. The ulcer presents clinically as a deep crater with or without undermining of adjacent tissue. Stage 4: stage 4 ulceration may be characterized by full thickness skin loss with extensive destruction, tissue necrosis, or damage to muscle, bone, or supporting structures (e.g., tendon, joint capsule, etc.).

Exemplary chronic wounds also include “decubitus ulcers.” Exemplary decubitus ulcer may arise as a result of prolonged and unrelieved pressure over a bony prominence that leads to ischemia. The wound tends to occur in patients who are unable to reposition themselves to off-load weight, such as paralyzed, unconscious, or severely debilitated persons. As defined by the U.S. Department of Health and Human Services, the major preventive measures include identification of high-risk patients; frequent assessment; and prophylactic measures such as scheduled repositioning, appropriate pressure-relief bedding, moisture barriers, and adequate nutritional status. Treatment options may include, for example, pressure relief, surgical and enzymatic debridement, moist wound care, and bacterial load control. Certain embodiments of the invention involve treating a chronic wound characterized by a decubitus ulcer or ulceration that results from prolonged, unrelieved pressure over a bony prominence that leads to ischemia.

Exemplary chronic wounds also include “arterial ulcers.” Arterial ulcers include those characterized by complete or partial arterial blockage, which may lead to tissue necrosis and/or ulceration. Signs of arterial ulcer can include, for example, pulselessness of the extremity; painful ulceration; small, punctuate ulcers that are usually well circumscribed; cool or cold skin; delayed capillary return time (briefly push on the end of the toe and release, normal color should return to the toe in about 3 seconds or less); atrophic-appearing skin (for example, shiny, thin, dry); and loss of digital and pedal hair.

Exemplary chronic wounds also include “venous ulcers.” Exemplary venous ulcers include the most common type of ulcer affecting the lower extremities and may be characterized by malfunction of the venous valve. The normal vein has valves that prevent the backflow of blood. When these valves become incompetent, the backflow of venous blood causes venous congestion. Hemoglobin from the red blood cells escapes and leaks into the extravascular space, causing the brownish discoloration commonly noted. It has been shown that the transcutaneous oxygen pressure of the skin surrounding a venous ulcer is decreased, indicating that there are forces obstructing the normal vascularity of the area. Lymphatic drainage and flow also plays a role in these ulcers. A venous ulcer can appear near the medial malleolus and usually occurs in combination with an edematous and indurated lower extremity; it may be shallow, not too painful, and may present with a weeping discharge from the affected site.

Exemplary chronic wounds also include “venous stasis ulcers.” Exemplary venous stasis ulcer are characterized by chronic passive venous congestion of the lower extremities that results in local hypoxia. One possible mechanism of pathogenesis of these wounds includes the impediment of oxygen diffusion into the tissue across thick perivascular fibrin cuffs. Another mechanism is that macromolecules leaking into the perivascular tissue trap growth factors needed for the maintenance of skin integrity. Additionally, the flow of large white blood cells slows due to venous congestion, occluding capillaries, becoming activated, and damaging the vascular endothelium to predispose to ulcer formation.

Exemplary chronic wounds further include “diabetic foot ulcers.” Diabetic patients with exemplary diabetic foot ulcer are prone to foot ulcerations due to both neurologic and vascular complications. Peripheral neuropathy can cause altered or complete loss of sensation in the foot and/or leg. Diabetic patients with advanced neuropathy lose all ability for sharp-dull discrimination. Any cuts or trauma to the foot may go completely unnoticed for days or weeks in a patient with neuropathy. A patient with advanced neuropathy can lose the ability to sense a sustained pressure insult and, as a result, tissue ischemia and necrosis may occur leading to, for example, plantar ulcerations. Additionally, microfractures in the bones of the foot, if unnoticed and untreated, may result in disfigurement, chronic swelling, and additional bony prominences. Microvascular disease is one of the significant complications for diabetics that may also lead to ulcerations.

Exemplary chronic wounds can include “traumatic ulcers.” Formation of exemplary traumatic ulcers may occur as a result of traumatic injuries to the body. These injuries include, for example, compromises to the arterial, venous, or lymphatic systems; changes to the bony architecture of the skeleton; loss of tissue layers—epidermis, dermis, subcutaneous soft tissue, muscle or bone; damage to body parts or organs and loss of body parts or organs.

Exemplary chronic wounds can include “burn ulcers” including, for example, ulceration that occur as a result of a burn injury, including a first degree burn (i.e., superficial, reddened area of skin); a second degree burn (a blistered injury site which may heal spontaneously after the blister fluid has been removed); a third degree burn (burn through the entire skin and usually require surgical intervention for wound healing); scalding (may occur from scalding hot water, grease or radiator fluid); a thermal burn (may occur from flames, usually deep burns); a chemical burn (may come from acid and alkali, usually deep burns); an electrical burn (either low voltage around a house or high voltage at work); an explosion flash (usually superficial injuries); and contact burns (usually deep and may occur from muffler tail pipes, hot irons, and stoves).

As used herein, a delayed or difficult to heal wound may include, for example, a wound that is characterized at least in part by 1) a prolonged inflammatory phase, 2) a slow forming extracellular matrix (ECM), and 3) a decreased rate of epithelialization.

As used herein, “fibrotic” diseases, disorders, or conditions include those mentioned herein, and further include acute and chronic, clinical or sub-clinical presentation, in which fibrogenic associated biology or pathology is evident. Fibrotic diseases, disorders, or conditions include diseases, disorders or conditions characterized, in whole or in part, by the excess production of fibrous material, including excess production of fibrotic material within the extracellular matrix, or the replacement of normal tissue elements by abnormal, non-functional, and/or excessive accumulation of matrix-associated components. Fibrotic diseases, disorders, or conditions include, for example, fibrogenic-related biology or pathology characterized by fibrosis.

Exemplary fibrotic diseases, disorders, and conditions include, for example, scleroderma (including morphea, generalized morphea, or linear scleroderma), kidney fibrosis (including glomerular sclerosis, renal tubulointerstitial fibrosis, progressive renal disease or diabetic nephropathy), cardiac fibrosis (e.g., myocardial fibrosis), pulmonary fibrosis (e.g., glomerulosclerosis pulmonary fibrosis, idiopathic pulmonary fibrosis, silicosis, asbestosis, interstitial lung disease, interstitial fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fascilitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, collagenous colitis, and acute fibrosis.

Fibrotic diseases, disorders, and conditions can also include contractures. Contractures, including post-operative contractures, refer to a permanent or long term reduction of range of motion due to tonic spasm or fibrosis, or to loss of normal tissue compliance, motion, or equilibrium (e.g., muscle, tendon, ligament, fascia, synovium, joint capsule, other connective tissue, or fat). In general, the condition of contracture may involve a fibrotic response with inflammatory components, both acute and chronic. Some of which may be associated with surgery, including a release procedure. Hereditary contractures such as Dupytren's contracture, Peyronie's disease, and Ledderhose's disease are also included.

Fibrosis can be either chronic or acute. Fibrotic conditions include excessive amounts of fibrous tissue, including excessive amounts of extracellular matrix accumulation within a tissue, forming tissue that causes dysfunction and, potentially, organ failure. Chronic fibrosis includes fibrosis of the major organs, most commonly lung, liver, kidney, and/or heart. Acute fibrosis (usually with a sudden and severe onset and of short duration) occurs typically as a common response to various forms of trauma including injuries, ischemic illness (e.g. cardiac scarring following heart attack), environmental pollutants, alcohol and other types of toxins, acute respiratory distress syndrome, radiation, and chemotherapy treatments. All tissues damaged by trauma can become fibrotic, particularly if the damage is repeated.

Response to injury involves coordinated and temporally regulated patterns of mediators and sequence of cellular events in tissues subsequent to injury. The initial injury triggers a coagulation cascade and an acute local inflammatory response followed by mesenchymal cell recruitment, proliferation, and matrix synthesis. Uncontrolled matrix accumulation, often involving aberrant cytokine pathways, can lead to fibrotic conditions or disorders. Progressive fibrosis in vital organs such as the lung, kidney, liver, heart, brain, and bone marrow, is both a major cause of illness and death.

### Adhesions

Within other aspects of the invention, methods are provided for treating, reducing the incidence or severity of, and/or preventing or retarding adhesions, surgical adhesions, and/or secondary surgical adhesions by administering to a patient an anti-connexin polynucleotide.

Adhesion formation is a complex process in which bodily tissues that are normally separate grow together. For example, post-operative adhesions have been reported to occur in about 60% to 90% of patients undergoing major gynecological surgery. Surgical trauma as a result of tissue (e.g., epithelial, connective, muscle, and nerve tissue) drying, ischemia, thermal injury, infection, or the presence of a foreign body, has long been recognized as a stimulus for tissue adhesion formation. These adhesions are a major cause of failed surgical therapy and are the leading cause of bowel obstruction and infertility. Other adhesion-treated complications include chronic pelvic pain, urethral obstruction, and voiding dysfunction.

Generally, adhesion formation is an inflammatory reaction in which factors are released, increasing vascular permeability and resulting in fibrinogen influx and fibrin deposition. This deposition forms a matrix that bridges the abutting tissues. Fibroblasts accumulate, attach to the matrix, deposit collagen, and induce angiogenesis. If this cascade of events can be prevented within 4 to 5 days following surgery, adhesion formation can be inhibited.

Secondary surgical adhesions may also form as a result of a corrective surgical procedure designed to correct and existing adhesion. The procedure may be a release or separation procedure.

A wide variety of animal models can be used to assess a particular therapeutic composition or treatment regimen for its therapeutic potential. Briefly, peritoneal adhesions have been observed to occur in animals as a result of inflicted severe damage that usually involves two adjacent surfaces. Injuries may be mechanical, due to ischemia, or as a result of the introduction of foreign material. Mechanical injuries include crushing of the bowel and stripping or scrubbing away the outer layers of bowel wall. Dividing major vessels to loops of the intestine induces ischemia. Foreign material that may be introduced into the area includes talcum, gauze sponges, toxic chemicals, bacteria, and feces.

Presently, typical animal models to evaluate prevention of formation of adhesions include the rabbit uterine horn model which involves the abrasion of the rabbit uterus, the rabbit uterine horn devascularization modification model which involves abrasion, devascularization of the uterus, and the rabbit cecal sidewall model which involves the excision of a patch of parietal peritoneum plus the abrasion of the cecum. Those and other reported evaluation models are described herein.

### Anti-Cadherin Agents

Anti-cadherin agents of the invention described herein are capable of modulating (e.g., blocking or inhibiting or downregulating) or affecting cadherin activity and function, cadherin complex formation and maintenance, adherens junction formation and maintenance, and cell-cell adhesion. Thus, certain anti-cadherin agents described herein modulate cellular adhesion (i.e., cell-to-cell adhesion). Certain anti-cadherin agents are adherens junction modulation agents. Such anti-connexin agents are generally targeted to messenger RNA (mRNA) molecules (or the genes encoding them) that, when translated, result in cadherin protein synthesis and localization to the cell membrane, where they are available for adherens junction formation. Other anti-cadherin agents interfere with cadherin complex and/or adherens junction formation. Thus, an anti-cadherin agents provided herein may directly or indirectly reduce coupling and communication between cells or reduce or block communication (or the transmission of molecules) between adjoining cells. Preferably, the cadherin is N-cadherin.

Any anti-cadherin agent that is capable of eliciting a desired modulation of cadherin activity, cadherin complex formation, and/or adherens junction formation may be used in practicing the invention. Such compounds include, for example, proteins and polypeptides, polynucleotides, and other organic compounds, and they may, for example, block the function or expression of adherens junctions in whole or in part, or down-regulate the production of one or more cadherin proteins, cadherin complexes, and/or adherens junctions in whole or in part.

Certain anti-cadherin agents provide downregulation of cadherin expression (for example, by downregulation of mRNA transcription or translation) or otherwise decrease or inhibit the activity of a cadherin protein, a cadherin complex, or adherens junctions. In the case of downregulation, this will have the effect of reducing direct cell-cell adhesion mediated by adherens junctions.

Examples of anti-cadherin agents include agents that decrease or inhibit expression or function of cadherin mRNA and/or protein or that decrease activity, expression, or formation of a cadherin protein species, cadherin complexes, or adherens junctions. Anti-cadherin agents include anti-cadherin polynucleotides, such as antisense polynucleotides and other polynucleotides (such as miRNAs and polynucleotides having siRNA or ribozyme functionalities), as well as antibodies and antigen-binding fragments thereof, and peptides and polypeptides, including peptidomimetics and peptide analogs that modulate cadherin or adherens junction activity or function, and deoxyribozymes. Anti-cadherin agents are preferred, particularly anti-N-cadherin agents.

Anti-Cadherin Polynucleotides

Anti-cadherin polynucleotides include connexin antisense polynucleotides as well as polynucleotides which have functionalities which enable them to down-regulate cadherin expression. Other suitable anti-cadherin polynucleotides include miRNAs, RNAi polynucleotides and siRNA polynucleotides. Anti-N-cadherin polynucleotides are preferred.

Synthesis of antisense polynucleotides and other polynucleotides that can serve as anti-cadherin polynucleotides, such as miRNA, RNAi, siRNA, and ribozyme polynucleotides as well as polynucleotides having modified and mixed backbones, is known to those of skill in the art. See e.g. Stein C. A. and Krieg A. M. (eds), Applied Antisense Oligonucleotide Technology, 1998 (Wiley-Liss). Methods of synthesizing desired antibodies and antigen-binding fragments, as well as desired peptides and polypeptides, including peptidomimetics and peptide analogs, are known to those of skill in the art. See e.g. Lihu Yang et al., Proc. Natl. Acad. Sci. U.S.A., 1; 95(18): 10836-10841 (Sep. 1, 1998); Harlow and Lane (1988) “Antibodies: A Laboratory Manuel” Cold Spring Harbor Publications, New York; Harlow and Lane (1999) “Using Antibodies” A Laboratory Manuel, Cold Spring Harbor Publications, New York.

According to one aspect, the downregulation of cadherin expression is based generally upon the antisense approach using antisense polynucleotides (such as DNA or RNA polynucleotides), and more particularly upon the use of antisense oligodeoxynucleotides (ODN). These polynucleotides (e.g., ODN) target mRNA molecules coding for the cadherin protein (s) to be downregulated. Typically the polynucleotides are single-stranded, but may be double-stranded.

The antisense polynucleotide may inhibit transcription and/or translation of a target cadherin protein species. Preferably, the polynucleotide is a specific inhibitor of transcription and/or translation from the cadherin gene or mRNA, and does not inhibit transcription and/or translation from other genes or mRNAs. The product binds to the cadherin gene or mRNA (i) 5′ to the coding sequence, and/or (ii) to the coding sequence, and/or (iii) 3′ to the coding sequence.

The antisense polynucleotide is generally antisense to a cadherin mRNA, preferably N-cadherin mRNA. Such a polynucleotide may be capable of hybridizing to the cadherin mRNA and can thus inhibit the expression of cadherin by interfering with one or more aspects of cadherin mRNA metabolism including transcription, mRNA processing, mRNA transport from the nucleus, translation, or mRNA degradation. While not wishing to be bound to a particular theory, the antisense polynucleotide typically hybridizes to the cadherin mRNA to form a duplex which can cause direct inhibition of translation and/or destabilization of the mRNA. Such a duplex may be susceptible to degradation by nucleases.

The antisense polynucleotide may hybridize to all or part of a cadherin mRNA. Typically, the antisense polynucleotide hybridizes to the ribosome binding region and/or the coding region of the cadherin mRNA. The polynucleotide may be complementary to all of or a region of the target cadherin mRNA. For example, the polynucleotide may be the exact complement of all or a part of a cadherin mRNA. However, absolute complementarity is not required and polynucleotides that have sufficient complementarity to form a duplex having a melting temperature of greater than about 5° C., 10° C., 20° C., 30° C., or 40° C. more than physiological temperature are particularly suitable for use in the present invention.

Thus the polynucleotide is typically a homologue of a sequence complementary to the target cadherin mRNA. The polynucleotide may be a polynucleotide which hybridizes to the cadherin mRNA under conditions of medium to high stringency, such as 0.03M sodium chloride and 0.03M sodium citrate at from about 50° C. to about 60° C.

For certain aspects, suitable polynucleotides are typically from about 6 to 40 nucleotides in length, for example. Preferably a polynucleotide may be from about 5 and about 100 nucleotides in length, preferably about 6 to about 40 nucleotides in length, preferably about 12 to about 35 nucleotides in length, or alternatively from about 12 to about 20 nucleotides in length or more preferably from about 18 to about 32 nucleotides in length. According to alternative embodiments, the polynucleotide is at least about 40, for example at least about 60 or at least about 80, nucleotides in length and up to about 100, about 200, about 300, about 400, about 500, about 1000, about 2000, or about 3000 or more nucleotides in length.

The connexin protein or proteins targeted by an anti-cadherin polynucleotide will be dependent upon the site at which downregulation is to be effected. This reflects the non-uniform make-up of adherens junction(s) at different sites throughout the body in terms of cadherin complex composition. The cadherin is one that naturally occurs in a human or animal in one aspect or naturally occurs in the tissue in which cadherin expression or activity is to be modulated, preferably decreased. The cadherin gene (including coding sequence) generally has homology with the coding sequence of one or more of the specific cadherins mentioned herein, such as homology with the cadherin coding sequence shown in Example 1, below. The cadherin is typically N-cadherin, E-cadherin, P-cadherin, cadherin 11, cadherin 12, a protocadherin protein, a desmoglein protein, or a desmocollin protein. Preferably, the cadherin N-cadherin and is expressed in the tissue to be treated.

Anti-cadherin polynucleotides include cadherin antisense polynucleotides as well as polynucleotides that have functionalities enabling them to downregulate cadherin expression. Other suitable anti-cadherin polynucleotides include RNAi polynucleotides and siRNA polynucleotides.

In many preferred embodiments, the antisense polynucleotides are targeted to the mRNA of one cadherin protein species only. Most preferably, this cadherin protein is N-cadherin. It is also contemplated that polynucleotides targeted to separate cadherin protein species be used in combination (for example 1, 2, 3, 4, or more different cadherin superfamily members can be targeted). For example, polynucleotides targeted to N-cadherin, and one or more other members of the cadherin superfamily (e.g., E-cadherin, P-cadherin, cadherin 11, cadherin 12, a protocadherin protein, a desmoglein protein, or a desmocollin protein) can be used in combination. Alternatively, the antisense polynucleotides of the invention may be part of compositions that may comprise polynucleotides targeted to more than one cadherin protein. Preferably, one of the cadherin proteins to which such polynucleotides are directed is N-cadherin. Thus, individual antisense polynucleotides may be specific to mRNA for a particular cadherin protein species, or may target mRNAs for two or more different cadherin protein species. Specific polynucleotides will generally target sequences in a cadherin gene or mRNA that are not conserved between cadherins, whereas non-specific anti-cadherin polynucleotides will target conserved sequences for various cadherins.

The polynucleotides and other agents, including deoxyribozymes, for use in the invention can be unmodified phosphodiester oligomers. Such oligodeoxynucleotides may vary in length. A 15-18 mer, 20-mer, and 30-mer polynucleotide has been found to be suitable. Many aspects of the invention are described with reference to oligodeoxynucleotides; however, it is understood that other suitable polynucleotides (such as RNA polynucleotides) can be used in these embodiments, as well.

The antisense polynucleotides and other agents, including deoxyribozymes, may be chemically modified. This may enhance their resistance to nucleases and may enhance their ability to enter cells. Such modifications are known in the art. For example, phosphorothioate oligonucleotides can be used. Other deoxynucleotide analogs include methylphosphonates, phosphoramidates, phosphorodithioates, N3′P5′-phosphoramidates, and oligoribonucleotide phosphorothioates and their 2′-O-alkyl analogs and 2′-O-methylribonucleotide methylphosphonates. Alternatively, mixed backbone oligonucleotides (“MBOs”) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloligoribonucleotides. Methods of preparing modified backbone and mixed backbone oligonucleotides are known in the art.

The precise sequence of the antisense polynucleotide(s) used in the invention will depend upon the target cadherin protein. In some embodiments, suitable cadherin antisense polynucleotides can include polynucleotides such as oligodeoxynucleotides. Suitable polynucleotides for the preparation of the combined polynucleotide compositions described herein include for example, polynucleotides to N-cadherin and polynucleotides for E-cadherin, P-cadherin, cadherin 11, cadherin 12, a protocadherin protein, a desmoglein protein, or a desmocollin protein.

Polynucleotides, including ODN's, directed to cadherin proteins can be selected in terms of their nucleotide sequence by any convenient, and conventional, approach. For example, the computer programs MacVector and OligoTech (from Oligos etc. Eugene, Oreg., USA) can be used. Once selected, the ODN's can be synthesized using an automated DNA synthesizer.

Polynucleotide Homologues

Homology and homologues are discussed herein (for example, the polynucleotide may be a homologue of a complement to a sequence in cadherin mRNA). Such a polynucleotide typically has at least about 70% homology, preferably at least about 80%, at least about 90%, at least about 95%, at least about 97% or at least about 99% homology with the relevant sequence, for example, over a region of at least about 15, at least about 20, at least about 25, at least about 30, at least about 40, at least about 50, or at least about 100 more contiguous nucleotides (of the homologous sequence).

Homology or sequence identity may be calculated based on any method in the art. For example, the UWGCG Package provides the BESTFIT program that can be used to calculate homology (Devereux, et al. (1984) Nucleic Acids Research 12, p387-395). The PILEUP and BLAST algorithms can also be used to calculate sequence identity or align sequences, for example, as described in Altschul, S. F. (1993), J Mol Evol 36: 290-300; Altschul, et al (1990), J Mol Biol 215: 403-10. Software for performing BLAST analyses is publicly available through the National Center for Biotechnology Information (http://www.ncbi.nlm.nih.gov/). The BLAST algorithm performs a statistical analysis of the similarity between two sequences; see e.g., Karlin and Altschul (1993) Proc. Natl. Acad. Sci. USA 90: 5873-5787. The homologous sequence typically differs from the relevant sequence by at least about (or by no more than about) 2, 5, 10, 15, 20, or more nucleotide differences (which may be substitutions, deletions, or insertions). These differences can be measured across any of the regions mentioned above in relation to calculating sequence identity or homology.

The homologous sequence typically hybridizes selectively to the original sequence at a level significantly above background. Selective hybridization is typically achieved using conditions of medium to high stringency (for example 0.03 M sodium chloride and 0.03 M sodium citrate at from about 50° C. to about 60° C.). However, such hybridization may be carried out under any suitable conditions known in the art (see Sambrook, et al. (1989), Molecular Cloning: A Laboratory Manual). For example, if high stringency is required, suitable conditions include 0.2×SSC at 60° C. If lower stringency is required, suitable conditions include 2×SSC at 60° C.

Peptide and Polypeptide Anti-Cadherin Agents

Cadherin binding proteins, including peptides, peptidomimetics, antibodies, antigen-binding antibody fragments, and the like, are also suitable modulators of adherens junctions.

Binding proteins include, for example, monoclonal antibodies, polyclonal antibodies, antibody fragments (including, for example, Fab, F(ab′)₂ and Fv fragments; single chain antibodies; single chain Fvs; and single chain binding molecules such as those comprising, for example, a binding domain, hinge, CH2 and CH3 domains, recombinant antibodies, and antibody fragments which are capable of binding an antigenic determinant (i.e., that portion of a molecule, generally referred to as an epitope) that makes contact with a particular antibody or other binding molecule. These binding proteins, including antibodies, anti-binding antibody fragments, and so on, may be chimeric or humanized or otherwise made to be less immunogenic in the subject to whom they are to be administered, and may be synthesized, produced recombinantly, or produced in expression libraries. Any binding molecule known in the art or later discovered is envisioned, such as those referenced herein and/or described in greater detail in the art. For example, binding proteins include not only antibodies, and the like, but also ligands, receptors, peptidomimetics, or other binding fragments or molecules (for example, produced by phage display) that bind to a target (e.g., a cadherin protein, another protein in a cadherin complex or adherens junction, or associated molecules). Fab fragments to N-cadherin extracellular domains have been reported to inhibit adherens junction formation. Meyer, et al. (1992) J. Cell Biol. 1992 October 1; 119(1): 179-189.

Binding molecules will generally have a desired specificity, including but not limited to binding specificity, and desired affinity. Affinity, for example, may be a Kₐ of greater than or equal to about 10⁴ M⁻¹, greater than or equal to about 10⁶ M⁻¹, greater than or equal to about 10⁷ M⁻¹, greater than or equal to about 10⁸ M⁻¹. Affinities of even greater than about 10⁸ M⁻¹ are suitable, such as affinities equal to or greater than about 10⁹ M⁻¹, about 10¹⁰ M⁻¹, about 10¹¹ M⁻¹, and about 10¹² M⁻¹. Affinities of binding proteins according to the present invention can be readily determined using conventional techniques, for example those described by Scatchard, et al., 1949 Ann. N.Y. Acad. Sci. 51: 660.

The extracellular domains of cadherin proteins contributed by two adjacent cells “dock” with each other to form complete gap junction channels. Reagents that interfere with the interactions of these extracellular domains can impair adherens junction formation and/or stability.

Anti-cadherin agents include peptides comprising an amino acid sequence corresponding to a cadherin domain motif from a cadherin protein (e.g., E-cadherin, N-cadherin, etc.). Other embodiments are directed to an anti-connexin agent that is a peptide having an amino acid sequence that comprises at least about 5, at least about 6, at least about 7, at least about 8, at least about 9, at least about 10, at least about 11, at least about 12, at least about 13, at least about 14, at least about 15, at least about 20, at least about 25, or at least about 30 contiguous amino acids encoded by a cadherin gene, for example, an N-cadherin gene as set forth in Example 1, below. In certain anti-connexin agents provided herein, the extracellular domains of N-cadherin may be used to develop the particular peptide sequences. The peptides need not have an amino acid sequence identical to those portions of naturally occurring N-cadherin, and conservative amino acid changes may be made such that the peptides retain binding activity or functional activity. Alternatively, peptides may target other regions of the extracellular domain.

Anti-cadherin peptides may comprise sequences corresponding to a portion of a cadherin extracellular domain with conservative amino acid substitutions such that peptides are functionally active anti-cadherin agents. Exemplary conservative amino acid substitutions include for example the substitution of a nonpolar amino acid with another nonpolar amino acid, the substitution of an aromatic amino acid with another aromatic amino acid, the substitution of an aliphatic amino acid with another aliphatic amino acid, the substitution of a polar amino acid with another polar amino acid, the substitution of an acidic amino acid with another acidic amino acid, the substitution of a basic amino acid with another basic amino acid, and the substitution of an ionizable amino acid with another ionizable amino acid.

Adherens Junction Modulation Agents

Certain anti-cadherin agents described herein are capable of modulation or affecting (e.g. blocking or inhibiting) adhesion between cells. Thus, certain adherens junction modulation agents described herein modulate cellular adhesion. As used herein, “adherens junction modulation agent” broadly includes any agent or compound that prevents, decreases, or modulate, in whole or in part, the activity, function, formation, or stability of an adherens junction. In certain embodiments, an adherens junction modulation agent prevents or decreases, in whole or in part, the function of an adherens junction. Exemplary adherens junction modulation agents may include, without limitation, polynucleotides, polypeptides (e.g. peptiditomimetics, antibodies, binding fragments thereof, and synthetic constructs), and other adherens junction modulating agents.

### Dosage Forms and Formulations and Administration

A therapeutically effective amount of each of the agents of the invention may be administered simultaneously, separately, or sequentially and in any order. The agents may be administered separately or as a fixed combination. When not administered as a fixed combination, preferred methods include the sequential administration of one or more anti-cadherin agents, alone or in combination with one or more other therapeutic agents, including other anti-cadherin agents, anti-connexin agents, anti-ZO-1 agents, and/or anti-osteopontin agents.

Where an anti-cadherin agent and other therapeutic agent are administered in combination, either or both are provided in amounts or doses that are less that those used when the agent or agents are administered alone, i.e., when they are not administered in combination, either physically or in the course of treatment of a wound. Such lesser amounts of agents administered are typically from about one-twentieth to about one-tenth the amount or amounts of the agent when administered alone, and may be about one-eighth the amount, about one-sixth the amount, about one-fifth the amount, about one-fourth the amount, about one-third the amount, and about one-half the amount when administered alone. Preferably, the agents are administered sequentially within at least about one-half hour of each other. The agents may also be administered with about one hour of each other, with about one day to about one week of each other, or as otherwise deemed appropriate.

The agents of the invention of the may be administered to a subject in need of treatment, such as a subject with any of the diseases or conditions mentioned herein. The condition of the subject can thus be improved. An anti-cadherin agent may thus be used in the treatment of the subject's body by therapy. They may be used in the manufacture of a medicament to treat any of the conditions mentioned herein.

The anti-cadherin agents of the invention are preferably used in the various compositions and methods of the invention in a substantially isolated form. It will be understood that the product may be mixed with carriers or diluents that will not interfere with the intended purpose of the product and still be regarded as substantially isolated. A product of the invention may also be in a substantially purified form, in which case it will generally comprise about 80%, 85%, or 90%, e.g. at least about 95%, at least about 98% or at least about 99% of the polynucleotide (or other anti-cadherin agent) or dry mass of the preparation.

Depending on the intended route of administration, the pharmaceutical products, pharmaceutical compositions, combined preparations and medicaments of the invention may, for example, take the form of solutions, suspensions, instillations, salves, creams, gels, foams, ointments, emulsions, lotions, paints, sustained release formulations, or powders, and typically contain about 0.1%-95% of active ingredient(s), preferably about 0.2%-70%. Other suitable formulations include pluronic gel-based formulations, carboxymethylcellulose(CMC)-based formulations, and hyroxypropylmethylcellulose(HPMC)-based formulations. Suitable formulations including pluronic gel, have for example about 10 to about 15 percent, about 15-20 percent, about 20-25 percent, and about 25-30 percent, suitably about 22 percent, pluronic gel. Other useful formulations include slow or delayed release preparations and instillations.

Gels or jellies may be produced using a suitable gelling agent including, but not limited to, gelatin, tragacanth, alginate, or a cellulose derivative and may include glycerol as a humectant, emollient, and preservative. Ointments are semi-solid preparations that consist of the active ingredient incorporated into a fatty, waxy, or synthetic base. Examples of suitable creams include, but are not limited to, water-in-oil and oil-in-water emulsions. Water-in-oil creams may be formulated by using a suitable emulsifying agent with properties similar, but not limited, to those of the fatty alcohols such as cetyl alcohol or cetostearyl alcohol and to emulsifying wax. Oil-in-water creams may be formulated using an emulsifying agent such as cetomacrogol emulsifying wax. Suitable properties include the ability to modify the viscosity of the emulsion and both physical and chemical stability over a wide range of pH. The water soluble or miscible cream base may contain a preservative system and may also be buffered to maintain an acceptable physiological pH.

Foam preparations may be formulated to be delivered from a pressurized aerosol canister, via a suitable applicator, using inert propellants. Suitable excipients for the formulation of the foam base include, but are not limited to, propylene glycol, emulsifying wax, cetyl alcohol, and glyceryl stearate. Potential preservatives include methylparaben and propylparaben.

Preferably the agents of the invention are combined with a pharmaceutically acceptable carrier or diluent to produce a pharmaceutical composition. Suitable carriers and diluents include isotonic saline solutions, for example phosphate-buffered saline. Suitable diluents and excipients also include, for example, water, saline, dextrose, glycerol, or the like, and combinations thereof. In addition, if desired substances such as wetting or emulsifying agents, stabilizing, or ph buffering agents may also be present.

The term “pharmaceutically acceptable carrier” refers to any pharmaceutical carrier that does not itself induce the production of antibodies harmful to the individual receiving the composition, and which can be administered without undue toxicity. Suitable carriers can be large, slowly metabolized macromolecules such as proteins, polysaccharides, polylactic acids, polyglycolic acids, polymeric amino acids, and amino acid copolymers.

Pharmaceutically acceptable salts can also be present, e.g., mineral acid salts such as hydrochlorides, hydrobromides, phosphates, sulfates, and the like; and the salts of organic acids such as acetates, propionates, malonates, benzoates, and the like.

Suitable carrier materials include any carrier or vehicle commonly used as a base for creams, lotions, gels, emulsions, lotions, or paints for topical administration. Examples include emulsifying agents, inert carriers including hydrocarbon bases, emulsifying bases, non-toxic solvents, or water-soluble bases. Particularly suitable examples include pluronics, HPMC, CMC and other cellulose-based ingredients, lanolin, hard paraffin, liquid paraffin, soft yellow paraffin or soft white paraffin, white beeswax, yellow beeswax, cetostearyl alcohol, cetyl alcohol, dimethicones, emulsifying waxes, isopropyl myristate, microcrystalline wax, oleyl alcohol, honey (including manuka honey), and stearyl alcohol.

Preferably, the pharmaceutically acceptable carrier or vehicle is a gel, suitably a nonionic polyoxyethylene-polyoxypropylene copolymer gel, for example, a Pluronic gel, preferably Pluronic F-127 (BASF Corp.). This gel is particularly preferred as it is a liquid at low temperatures but rapidly sets at physiological temperatures, which confines the release of the agent to the site of application or immediately adjacent that site. Pharmaceutical carriers also include liposomes, nanosomes, and the like.

An auxiliary agent such as casein, gelatin, albumin, glue, sodium alginate, carboxymethylcellulose, methylcellulose, hydroxyethylcellulose, or polyvinyl alcohol may also be included in the formulation of the invention.

Other suitable formulations include pluronic gel-based formulations, carboxymethylcellulose(CMC)-based formulations, and hyroxypropylmethylcellulose(HPMC)-based formulations. The composition may be formulated for any desired form of delivery, including topical, instillation, parenteral, intramuscular, subcutaneous, or transdermal administration. Other useful formulations include slow or delayed release preparations.

Transdermal delivery can be carried out by methods known in the art or later discovered, including, for example, methods directed to 1) the use of chemical penetration enhancers or skin enhancers; 2) liposome-mediated delivery; 3) iontophoresis; 4) electroporation; 5) sonophoresis; 6) mechanical (e.g., microporation) devices. Exemplary methods suitable for transdermal delivery of the agents disclosed herein can include, for example, methods directed to enhancing the transport of material across the skin pores by increasing the rate of transport across existing pores or by amplifying the number of available skin pores through the creation of artificial pores.

Transdermal delivery can be carried out by the use of chemical or penetration enhancers, including for example, an pharmaceutically acceptable oil of vegetable, nut, synthetic or animal origin including emu oil, ethoxylated oil, PEG, linoleic acid, ethanol, 1-methanol, and/or agents which delipidize the stratum corneum. Suitable oils include meadowfoam oil, castor oil, jojoba oil, corn oil, sunflower oil, sesame oil, and emu oil, all of which may be optionally ethoxylated. Exemplars include those as described in U.S. Pat. Nos. 7,291,591, 7,201,919, 7,052,715, 7,033,998, 6,946,144; 6,951,658, 6,759,056, 6,720,001, 6,224,853, 5,695,779, and 6,750,291. In addition, transdermal patches can also be adapted for delivery of dry powder or lyophilized drugs, and exemplars include those described in U.S. Pat. No. 5,983,135.

Transdermal delivery can be carried out by liposome mediated delivery methods (e.g., delivery facilitated by application of lipophilic membrane active agents). Suitable exemplars may include those described in U.S. Pat. Nos. 5,910,306, 5,718,914, and 5,064,655.

Transdermal delivery systems can also be employed in conjunction with a wide variety of iontophoresis or electrotransport systems. Illustrative electrotransport drug delivery systems are disclosed in U.S. U.S. Pat. Nos. 5,147,296, 5,080,646, 5,169,382, and 5,169383.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may be achieved in various manners.

Transdermal delivery can be carried out by iontophoretic methods (e.g., delivery facilitated by application of low level electrical field to the skin over time). Suitable exemplars may include those described in U.S. Pat. Nos. 6,731,987, 6,391,015, 6553255, 4940456, 5681580, and 6248349.

Also, transdermal delivery can be carried out by electroporation methods (e.g., delivery facilitated by brief application of high voltage pulse to create transient pores in the skin). Suitable exemplars may include U.S. Pat. Nos. 7,008,637, 6,706,032, 6692456, 6587705, 6512950, 6041253, 5968006, and 5749847.

Transdermal delivery can be carried out by sonophoresis methods (e.g., delivery facilitated by application of pulses of low frequency ultrasound to increase skin permeability). Suitable exemplars may include U.S. Pat. Nos. 7,232,431, 7,004,933, 6842641, 6868286, 6712805, 6575956, 6491657, 6487447, 623499, and 6190315.

Transdermal delivery can be carried out by methods comprising the use of mechanical devices and/or creation of artificial micropores or microchannels (e.g., microprojections) by inducing mechanical alterations or disruptions in the structural elements, thermal stability properties, membrane fluidity and integrity of the dermal architecture and substructures. Suitable exemplars may include MicroPor (Altea Therapeutics), MacroFlux (Alza Corporation), as well as those as described in U.S. Pat. Nos. 6,893,655, 6,730,318, 5,484,604, 5,362,308, 5,320,850, and 5,279,544, and US re-examination certificate RE35474.

Other Suitable Formulations are are Formulations that May be Inhaled.

Where the anti-cadherin agent (and/or other therapeutic agent(s), if any) is a nucleic acid, such as a polynucleotide, uptake of nucleic acids by mammalian cells is enhanced by several known transfection techniques for example those including the use of transfection agents. Such techniques may be used with certain anti-connexin agents, including polynucleotides. The formulation that is administered may contain such transfection agents. Examples of these agents include cationic agents (for example calcium phosphate and DEAE-dextran) and lipofectants (for example Lipofectam™ and Transfectam™), and surfactants.

Where the anti-cadherin agent (and/or other therapeutic agent(s), if any) comprises a polynucleotide, conveniently, the formulation further includes a surfactant to assist with polynucleotide cell penetration or the formulation may contain any suitable loading agent. Any suitable non-toxic surfactant may be included, such as DMSO. Alternatively a transdermal penetration agent such as urea may be included.

The effective dose for a given subject or condition preferably lies within the dose that is therapeutically effective for at least 50% of the population, and that exhibits little or no toxicity at this level.

The effective dosage of each of the anti-cadherin agent (and/or other therapeutic agent(s), if any) employed in the methods and compositions of the invention may vary depending on a number of factors including the particular anti-connexin agent or agents employed, the combinational partner (if any), the mode of administration, the frequency of administration, the condition being treated, the severity of the condition being treated, the route of administration, the needs of a patient sub-population to be treated or the needs of the individual patient which different needs can be due to age, sex, body weight, relevant medical condition specific to the patient.

The dose at which an anti-cadherin agent (and/or other therapeutic agent(s), if any) is administered to a patient will depend upon a variety of factors such as the age, weight and general condition of the patient, the condition that is being treated, and the particular anti-connexin agent that is being administered.

A suitable therapeutically effective dose of an anti-cadherin agent (and/or other therapeutic agent(s), if any) may be from about 0.001 to about 1 mg/kg body weight such as about 0.01 to about 0.4 mg/kg body weight. A suitable dose may however be from about 0.001 to about 0.1 mg/kg body weight such as about 0.01 to about 0.050 mg/kg body weight.

Therapeutically effective doses of anti-cadherin agent(s) (and/or other therapeutic agent(s), if any) from about 1 to 100, 100-200, 100- or 200-300, 100- or 200- or 300-400, and 100- or 200- or 300- or 400-500 micrograms are appropriate. Doses from about 1-1000 micrograms are also appropriate. Doses up to 2 milligrams may also be used. Doses are adjusted appropriately when the anti-cadherin agent(s) (and/or other therapeutic agent(s), if any) are provided in the form of a dressing, typically upward to maintain the desired total dose administration.

Alternatively, in the case of anti-cadherin oligonucleotides or anti-cadherin proteins or peptides (and/or other therapeutic agent(s), if any), the dosage of each of the agents in the compositions may be determined by reference to the composition's concentration relative to the size, length, depth, area, or volume of the area to which it will be applied. For example, in certain topical applications, dosing of the pharmaceutical compositions may be calculated based on mass (e.g. grams) of or the concentration in a pharmaceutical composition (e.g. μg/ul) per length, depth, area, or volume of the area of application. Useful doses range from about 1 to about 10 micrograms per square centimeter of wound size. Certain doses will be about 1-2, about 1-5, about 2-4, about 5-7, and about 8-10 micrograms per square centimeter of wound size. Other useful doses are greater than about 10 micrograms per square centimeter of wound size, including at least about 15 micrograms per square centimeter of wound size, at least about 20 micrograms per square centimeter of wound size, at least about 25 micrograms per square centimeter of wound size, about 30 micrograms per square centimeter of wound size, at least about 35 micrograms per square centimeter of wound size, at least about 40 micrograms per square centimeter of wound size, at least about 50 micrograms per square centimeter of wound size, and at least about 100 to at least about 150 micrograms per square centimeter of wound size. Other doses include about 150-200 micrograms per square centimeter, about 200-250 micrograms per square centimeter, about 250-300 micrograms per square centimeter, about 300-350 micrograms per square centimeter, about 350-400 micrograms per square centimeter, and about 400-500 micrograms per square centimeter, and 500-1000 micrograms per square centimeter, and at least about 600-1000 micrograms per square centimeter.

In certain embodiments, the anti-cadherin agent composition (and/or other therapeutic agent composition(s), if any) may be applied at about 0.01 micromolar (μM) or 0.05 μM to about 200 μM, or up to 300 μM or up to 400, 500, 600, 700, 800, 900 μM or up to 1000 μM or up to 2000 μM or up to 3200 μM or more final concentration at the treatment site and/or adjacent to the treatment site, and any doses and dose ranges within these dose numbers. Preferably, antisense polynucleotide compositions are applied at about 0.05 μM to about 100 μM or more final concentration, more preferably, the anti-cadherin polynucleotide composition is applied at about 1.0 μM to about 50 μM final concentration, and more preferably, at about 5-10 μM to about 30-50 μM final concentration. Additionally, the combined anti-cadherin agent composition is applied at about 8 μM to about 20 μM final concentration, and alternatively the anti-cadherin agent composition is applied at about 10 μM to about 20 μM final concentration, or at about 10 to about 15 μM final concentration. In certain other embodiments, the anti-cadherin agent composition is applied at about 10 μM final concentration. In yet another embodiment, the anti-cadherin agent composition is applied at about 1-15 μM final concentration. In other embodiments, the anti-cadherin agent is applied at about a 20 μM, 30 μM, 40 μM, 50 μM, 60 μM, 70 μM, 80 μM, 90 μM, 100 μM., 10-200 μM, 200-300 μM, 300-400 μM, 400-500 μM, 500-600 μM, 600-700 μM, 700-800 μM, 800-900 μM, 900-1000 or 1000-1500 μM, or 1500 μM-2000 μM or 2000 μM-3000 μM or greater.

Anti-cadherin dose amounts include, for example, about 0.1-1, 1-2, 2-3, 3-4, or 4-5 micrograms (μg), from about 5 to about 10 μg, from about 10 to about 15 μg, from about 15 to about 20 μg, from about 20 to about 30 μg, from about 30 to about 40 μg, from about 40 to about 50 μg, from about 50 to about 75 μg, from about 75 to about 100 μg, from about 100 μg to about 250 μg, and from 250 μg to about 500 μg. Dose amounts from 0.5 to about 1.0 milligrams or more or also provided, as noted above. Dose volumes will depend on the size of the site to be treated, and may range, for example, from about 25-100 μL to about 100-200 μL, from about 200-500 μL to about 500-1000 μL. Milliliter doses are also appropriate for larger treatment sites.

Still other dosage levels between about 1 nanogram (ng)/kg and about 1 mg/kg body weight per day of each of the agents described herein. In certain embodiments, the dosage of each of the subject compounds will generally be in the range of about 1 ng to about 1 microgram per kg body weight, about 1 ng to about 0.1 microgram per kg body weight, about 1 ng to about 10 ng per kg body weight, about 10 ng to about 0.1 microgram per kg body weight, about 0.1 microgram to about 1 microgram per kg body weight, about 20 ng to about 100 ng per kg body weight, about 0.001 mg to about 0.01 mg per kg body weight, about 0.01 mg to about 0.1 mg per kg body weight, or about 0.1 mg to about 1 mg per kg body weight. In certain embodiments, the dosage of each of the subject compounds will generally be in the range of about 0.001 mg to about 0.01 mg per kg body weight, about 0.01 mg to about 0.1 mg per kg body weight, about 0.1 mg to about 1 mg per kg body weight. If more than one anti-cadherin agent is used, the dosage of each anti-cadherin agent need not be in the same range as the other. For example, the dosage of one anti-cadherin agent may be between about 0.01 mg to about 10 mg per kg body weight, and the dosage of another anti-cadherin (or other therapeutic agent) may be between about 0.1 mg to about 1 mg per kg body weight.

All doses and dose ranges referenced herein are applicable, for example, to polynucleotide therapeutic, including anti-cadherin agents that comprise oligonucleotides. These dose ranges are also applicable, for example, to therapeutic agents, including anti-cadherin agents, that comprise proteins and peptides, as well as mimetic peptides and peptidomimetics.

Conveniently, an anti-cadherin agent is administered in a sufficient amount to downregulate expression of a cadherin protein, or to modulate adherens junction formation or stability for at least about 0.5 to 1 hour, at least about 1-2 hours, at least about 2-4 hours, at least about 4-6 hours, at least about 6-8 hours, at least about 8-10 hours, at least about 12 hours, or at least about 24 hours post-administration.

The dosage of each of the anti-cadherin agent(s) in the compositions and methods of the subject invention may also be determined by reference to the concentration of the composition relative to the size, length, depth, area or volume of the area to which it will be applied. For example, in certain topical and other applications, e.g., instillation, dosing of the pharmaceutical compositions may be calculated based on mass (e.g. micrograms) of or the concentration in a pharmaceutical composition (e.g. ng/nl) per length, depth, area, or volume of the area of application.

The same dose amounts and frequencies and administrations, described above and below herein, are useful for anti-ZO-1 agents.

As noted herein, the doses of an anti-cadherin polynucleotide, peptide, or peptidomimetic administered in combination, or other anti-cadherin agents (and/or other therapeutic agents) administered in combination with either or both, can be adjusted down from the doses administered when given alone. The combined use of several agents may reduce the required dosage for any individual agent because the onset and duration of effect of the different agents may be complementary. In a preferred embodiment, the combined use of two or more anti-cadherin agents has an additive, synergistic, or super-additive effect. In some cases, the combination of one or more anti-cadherin agents and/or one or more other therapeutic agents in combination with either or both, have an additive effect. In other cases, the combination can have greater-than-additive effect. Such an effect is referred to herein as a “supra-additive” effect, and may be due to synergistic or potentiated interaction.

The term “supra-additive promotion of wound healing” refers to a mean wound healing produced by administration of a combination of one or more anti-cadherin agents with one or more therapeutic agents administered in combination with either or both, is statistically significantly higher than the sum of the decrease in adhesion formation by the individual administration of either of the agents alone. Whether the result is “statistically significantly higher” than the expected additive value of the individual compounds may be determined by a variety of statistical methods as described herein and/or known by one of ordinary skill in the art. The term “synergistic” refers to a type of supra-additive inhibition in which, for example, both an anti-cadherin polynucleotide and anti-cadherin peptide or peptidomimetic, or other anti-cadherin agents administered in combination with either or both, individually have the ability to prevent or decrease adhesion formation, for example. The term “potentiated” refers to type of supra-additive effect in which one of the anti-cadherin polynucleotide(s), peptide(s), or peptidomimetic(s), or other therapeutic agent(s) administered in combination individually has the increased ability to prevent or decrease adhesion formation, for example.

In general, potentiation may be assessed by determining whether the combination treatment produces a mean decrease, by way of example, in adhesion formation in a treatment group that is statistically significantly supra-additive when compared to the sum of the mean decrease in adhesion formation produced by the individual treatments in their treatment groups respectively. The mean decrease in adhesion formation, for example, may be calculated as the difference between control group and treatment group mean decrease in adhesion formation. The fractional decrease in adhesion formation, for example, “fraction affected” (Fa), may be calculated by dividing the treatment group mean decrease in adhesion formation by control group mean decrease in adhesion formation. Testing for statistically significant potentiation requires the calculation of Fa for each treatment group. The expected additive Fa for a combination treatment may be taken to be the sum of mean Fas from groups receiving either element of the combination. The Two-Tailed One-Sample T-Test, for example, may be used to evaluate how likely it is that the result obtained by the experiment is due to chance alone, as measured by the p-value. A value of less than 0.05 is considered statistically significant, that is, not likely to be due to chance alone. Thus, Fa for the combination treatment group must be statistically significantly higher than the expected additive Fa for the single element treatment groups to deem the combination as resulting in a potentiated supra-additive effect.

Whether a synergistic effect results from a combination treatment may be evaluated by the median-effect/combination-index isobologram method (Chou, T., and Talalay, P. (1984) Ad. Enzyme Reg. 22:27-55). In this method, combination index (CI) values are calculated for different dose-effect levels based on parameters derived from median-effect plots of an anti-osteopontin or anti-connexin agent alone, for example, the one or more agents alone, and the combination of the two at fixed molar ratios. CI values of & 1t; 1 indicate synergy, CI-1 indicates an additive effect, and CP1 indicates an antagonistic effect. This analysis may be performed using computer software tools, such as CalcuSyn, Windows Software for Dose Effect Analysis (Biosoft(D, Cambridge UK).

Any method known or later developed in the art for analyzing whether a supra-additive effect exists for a combination therapy is contemplated for use in screening for suitable anti-cadherin agents for use in combination with other anti-cadherin or other therapeutic agents.

In another preferred embodiment, the combined use of one or more anti-cadherin agents, particularly anti-cadherin polynucleotides, and one or more anti-cadherin peptides or peptidomimetics reduces the effective dose of any such agent compared to the effective dose when said agent administered alone. In certain embodiments, the effective dose of the agent when used in combination is about 1/15 to about ½, about 1/10 to about ⅓, about ⅛ to about ⅙, about ⅕, about ¼, about ⅓ or about ½ the dose of the agent when used alone.

In another preferred embodiment, the combined use of one or more anti-cadherin and one or more anti-cadherin peptides or peptidomimetics, or other therapeutic agents in combination with either or both, reduces the frequency in which said agent is administered compared to the frequency when said agent is administered alone. Thus, these combinations allow the use of lower and/or fewer doses of each agent than previously required to achieve desired therapeutic goals.

The doses may be administered in single or divided applications. The doses may be administered once, or application may be repeated. Typically, application will be repeated weekly until, for example, wound healing is promoted, or a repeat application may be made in the event that, for example, wound healing slows or is stalled. Doses may be applied 3-7 days apart, or more. In the case of a chronic wound, for example, repeat applications may be made, for example, weekly, or bi-weekly, or monthly or in other frequency for example if and when, for example, wound healing slows or is stalled. For some indications, such as certain ocular uses, more frequent dosing, up to hourly may employed.

In combination therapies, the anti-cadherin agent(s), alone or in conjunction with one or more therapeutic agents, can be administered by the same or different routes. The various agents can be administered separately at different times during the course of therapy, or concurrently in divided or single combination forms.

In some combination therapy embodiments, the anti-cadherin agent or anti_ZO-1 agent is administered in one composition and the second therapeutic agent, be it a second anti-cadherin agent, an anti-connexin agent, and/or an anti-osteopontin agent, is administered in a second composition. In some of these embodiments, the first composition is administered before the second composition. In other embodiments, the first composition is administered after the second composition. In still other embodiments, the first composition is administered before and after the second composition. In yet other embodiments, the second composition is administered before and after the first composition. In further such embodiments, the first composition is administered about the same time as the second composition.

Preferably one or more anti-cadherin agents and/or anti_ZO-1 agents is delivered by topical administration (peripherally or directly to a site), including but not limited to topical administration using solid supports (such as dressings and other matrices) and medicinal formulations (such as gels, mixtures, suspensions and ointments). In one embodiment, the solid support comprises a biocompatible membrane or insertion into a treatment site. In another embodiment, the solid support comprises a dressing or matrix. In one embodiment of the invention, the solid support composition may be a slow release solid support composition in which the anti-cadherin agent, alone or in admixture or combination with one or more additional therapeutic agents, is dispersed in a slow release solid matrix such as a matrix of alginate, collagen, or a synthetic bioabsorbable polymer. Preferably, the solid support composition is sterile or low bio-burden. In one embodiment, a wash solution comprising two or more anti-connexin agents can be used.

The delivery of a formulation of the invention comprising one or more active ingredients, over a period of time, in some instances for about 1-2 hours, about 2-4 hours, about 4-6 hours, about 6-8, or about 24 hours or longer, may be a particular advantage in more severe injuries or conditions.

While the delivery period will be dependent upon both the site at which the cadherin or cadherin complex modulation is to be induced and the therapeutic effect which is desired, continuous or slow-release delivery for about 0.5-1 hour, about 1-2 hours, about 2-4 hours, about 4-6 hours, about 6-8, or about 24 hours or longer is provided. In accordance with the present invention, this is achieved by inclusion of one or more anti-cadherin agent species, alone or in combination, in a formulation together with a pharmaceutically acceptable carrier or vehicle, particularly in the form of a formulation for continuous or slow-release administration.

As noted, the one or more agents of the invention may be administered before, during, or immediately following wounding, for example, or following a procedure likely or suspected to result in a scar, an adhesion, or fibrosis, for example, or within about 180, about 120, about 90, about 60, or about 30 days, but preferably within about 10, about 9, about 8, about 7, about 6, about 5, about 4, about 3, or about 2 days or less, and most preferably within about 24, about 12, about 10, about 9, about 8, about 7, about 6, about 5, about 4, about 3, about 2 hours or within about 60, about 45, about 30, about 15, about 10, about 5, about 4, about 3, about 2, about 1 minute following wounding or following a procedure likely or suspected to result in an adhesion, for example. The one or more agents of the invention may also be administered before and/or during a procedure likely or suspected to result in an adhesion, for example.

The agents and agent combinations of the invention can be administered in any manner that achieves a desired result. Preferred methods include peritubular administration (either direct application at the time of surgery or with endoscopic, ultrasound, CT, MRI, or fluoroscopic guidance); “coating” the surgical implant; and placement of a drug-eluting polymeric implant at the surgical site. In a preferred embodiment, 0.5% to 20% anti-cadherin agent(s) by weight is loaded into a polymeric carrier (as described in the following examples) and applied to the peritubular (mesenteric) surface as a “paste”, “film”, or “wrap” which releases the drug over a period of time such that the incidence of surgical adhesions is reduced. During endoscopic procedures, the polymer preparation may be applied as a “spray”, via delivery ports in the endoscope, to the mesentery of the abdominal and pelvic organs manipulated during the operation. In a particularly preferred embodiment, the peritubular composition is about 0.1% to about 5% active ingredient by weight. In another preferred embodiment, a polymeric coating containing about 0.1% to about 20% or more of active agent(s) is applied to the surface of the surgical implant (e.g., breast implant, artificial joint, vascular graft, etc.) to prevent encapsulation/inappropriate scarring, for example, in the vicinity of the implant. In yet another preferred embodiment, a polymeric implant containing about 0.01% to about 20% or more of active agent or agents by weight is applied directly to the surgical site (e.g., directly into the sinus cavity, chest cavity, abdominal cavity, or at the operative site during neurosurgery) such that adhesion formation, for example, is prevented or reduced. In one embodiment, one or more active agents can be administered via fluoroscopically guided intra-articular injection.

In another embodiment, lavage fluid containing about 1 to about 100 μg/cm² (preferably about 10 to about 50 μg/cm²) of an anti-cadherin agent(s), would be used at the time of or immediately following surgery and administered during surgery or intraperitoneally, by a physician. In all of the embodiments, other anti-cadherin agents and/or anti_ZO-1 agents, alone or in combination with other therapeutic agents, would be administered at equivalent doses adjusted for potency and tolerability of the agent.

The routes of administration and dosages described herein are intended only as a guide since a skilled physician will determine the optimum route of administration and dosage for any particular patient and condition.

Any of the agents and methods of treating a subject having a disease, disorder or condition referenced or described herein and treating subjects before or following a surgical procedure may utilize the administration of any of the doses, dosage forms, formulations, and/or compositions herein described.

Dressings and Matrices

In one aspect, one or more active agents are provided in the form of a dressing or matrix. In certain embodiments, the one or more agents of the invention are provided in the form of a liquid, semi solid or solid composition for application directly, or the composition is applied to the surface of, or incorporated into, a solid contacting layer such as a dressing gauze or matrix. The dressing composition may be provided for example, in the form of a fluid or a gel. One or more active agents may be provided in combination with conventional pharmaceutical excipients for topical application. Suitable carriers include: Pluronic gels, Poloxamer gels, Hydrogels containing cellulose derivatives, including hydroxyethyl cellulose, hydroxymethyl cellulose, carboxymethyl cellulose, hydroxypropylmethyl cellulose and mixtures thereof; and hydrogels containing polyacrylic acid (Carbopols). Suitable carriers also include creams/ointments used for topical pharmaceutical preparations, e.g., creams based on cetomacrogol emulsifying ointment. The above carriers may include alginate (as a thickener or stimulant), preservatives such as benzyl alcohol, buffers to control pH such as disodium hydrogen phosphate/sodium dihydrogen phosphate, agents to adjust osmolarity such as sodium chloride, and stabilizers such as EDTA.

In addition to the biological matrices previously mentioned, suitable dressings or matrices may include, for example, the following with one or more anti-cadherin agents (or other active agents to be administered alone or in combination therewith):

1) Absorptives: suitable absorptives may include, for example, absorptive dressings, which can provide, for example, a semi-adherent quality or a non-adherent layer, combined with highly absorptive layers of fibers, such as for example, cellulose, cotton or rayon. Alternatively, absorptives may be used as a primary or secondary dressing.

2) Alginates: suitable alginates include, for example, dressings that are non-woven, non-adhesive pads and ribbons composed of natural polysaccharide fibers or xerogel derived from seaweed. Suitable alginates dressings may, for example, form a moist gel through a process of ion exchange upon contact with exudate. In certain embodiments, alginate dressings are designed to be soft and conformable, easy to pack, tuck or apply over irregular-shaped areas. In certain embodiments, alginate dressings may be used with a second dressing.

3) Antimicrobial Dressings: suitable antimicrobial dressings may include, for example, dressings that can facilitate delivery of bioactive agents, such as, for example, silver and polyhexamethylene biguanide (PHMB), to maintain efficacy against infection, where this is needed or desirable. In certain embodiments, suitable antimicrobial dressings may be available as for example, as sponges, impregnated woven gauzes, film dressings, absorptive products, island dressings, nylon fabric, non-adherent barriers, or a combination of materials.

4) Biological & Biosynthetics: suitable biological dressings or biosynthetic dressings may include, for example, gels, solutions or semi-permeable sheets derived from a natural source, e.g., pigs or cows. In certain embodiments, a gel or solution is applied to the treatment site and covered with a dressing for barrier protection. In another embodiment, a biological-based (e.g., pig intestinal mucosa or bladder tissue) or biosynthetic-based sheet is placed in situ which may act as membrane, remaining in place after a single application, or the may be biological dressings or biosynthetic dressings may be prepared in advance to include one or more, preferably two, anti-cadherin agents.

5) Collagens: suitable collagen dressings may include, for example, gels, pads, particles, pastes, powders, sheets or solutions derived from for example, bovine, porcine or avian sources or other natural sources or donors. In certain embodiments, the collagen dressing may interact with treatment site exudate to form a gel. In certain embodiments, collagen dressing may be used in combination with a secondary dressing.

6) Composites: suitable composite dressings may include, for example, dressings that combine physically distinct components into a single product to provide multiple functions, such as, for example, a bacterial barrier, absorption, and adhesion. In certain embodiment, the composite dressings are comprised of, for example, multiple layers and incorporate a semi- or non-adherent pad. In certain embodiment, the composite may also include for example, an adhesive border of non-woven fabric tape or transparent film. In certain other embodiment, the composite dressing may function as for example, either a primary or a secondary dressing and in yet another embodiment, the dressing may be used in combination with topical pharmaceutical composition.

7) Contact Layers: suitable contact layer dressings may include, for example, thin, non-adherent sheets placed on an area to protect tissue from for example, direct contact with other agents or dressings applied to the treatment site. In certain embodiments, contact layers may be deployed to conform to the shape of the area of the treatment site and are porous to allow exudate to pass through for absorption by an overlying, secondary dressing. In yet another embodiment, the contact layer dressing may be used in combination with topical pharmaceutical composition.

8) Elastic Bandages: suitable elastic bandages may include, for example, dressings that stretch and conform to the body contours. In certain embodiment, the fabric composition may include for example, cotton, polyester, rayon, or nylon. In certain other embodiments, the elastic bandage may for example, provide absorption as a second layer or dressing, to hold a cover in place, to apply pressure or to cushion a treatment site.

9) Foams: suitable foam dressings may include, for example, sheets and other shapes of foamed polymer solutions (including polyurethane) with small, open cells capable of holding fluids. Exemplary foams may be for example, impregnated or layered in combination with other materials. In certain embodiment, the absorption capability may be adjusted based on the thickness and composition of the foam. In certain other embodiments, the area in contact with the treatment site may be non-adhesive for easy removal. In yet another embodiment, the foam may be used in combination with an adhesive border and/or a transparent film coating that can serve as an anti-infective barrier.

10) Gauzes & Non-Woven dressings: suitable gauze dressings and woven dressings may include, for example, dry woven or non-woven sponges and wraps with varying degrees of absorbency. Exemplary fabric composition may include, for example, cotton, polyester, or rayon. In certain embodiment, gauzes and non-woven dressing may be available sterile or non-sterile in bulk and with or without an adhesive border. Exemplary gauze dressings and woven dressings may be used for cleansing, packing and covering a variety of treatment sites.

11) Hydrocolloids: suitable hydrocolloid dressings may include, for example, wafers, powders or pastes composed of gelatin, pectin, or carboxymethylcellulose. In certain embodiment, wafers are self-adhering and available with or without an adhesive border and in a wide variety of shapes and sizes. Exemplary hydrocolloids are useful on areas that require contouring. In certain embodiments, powders and pastes hydrocolloids may use used in combination with a secondary dressing.

12) Hydrogels (Amorphous): suitable amorphous hydrogel dressings may include, for example, formulations of water, polymers and other ingredients with no shape, designed to donate moisture and to maintain a moist healing environments and or to rehydrate the treatment site. In certain embodiment, hydrogels may be used in combination with a secondary dressing cover.

13) Hydrogels: Impregnated Dressings: suitable impregnated hydrogel dressings may include, for example, gauzes and non-woven sponges, ropes and strips saturated with an amorphous hydrogel. Amorphous hydrogels may include for example, formulations of water, polymers and other ingredients with no shape, designed to donate moisture to a dry treatment site and to maintain a moist healing environment.

14) Hydrogel Sheets: suitable hydrogel sheets may include for example, three-dimensional networks of cross-linked hydrophilic polymers that are insoluble in water and interact with aqueous solutions by swelling. Exemplary hydrogels are highly conformable and permeable and can absorb varying amounts of drainage, depending on their composition. In certain embodiment, the hydrogel is non-adhesive against the treatment site or treated for easy removal.

15) Impregnated Dressings: suitable impregnated dressings may include, for example, gauzes and non-woven sponges, ropes and strips saturated with a solution, an emulsion, oil, gel or some other pharmaceutically active compound or carrier agent, including for example, saline, oil, zinc salts, petrolatum, xeroform, and scarlet red as well as the compounds described herein.

16) Silicone Gel Sheets: suitable silicone gel sheet dressings may include, for example, soft covers composed of cross-linked polymers reinforced with or bonded to mesh or fabric.

17) Solutions: suitable liquid dressings may include, for example, mixtures of multiprotein material and other elements found in the extracellular matrix. In certain embodiment, exemplary solutions may be applied to the treatment site after debridement and cleansing and then covered with an absorbent dressing or a nonadherent pad.

18) Transparent Films: suitable transparent film dressings may include polymer membranes of varying thickness coated on one side with an adhesive. In certain embodiments, transparent films are impermeable to liquid, water and bacteria but permeable to moisture vapor and atmospheric gases. In certain embodiments, the transparency allows visualization of the treatment site.

19) Fillers: suitable filler dressings may include, for example, beads, creams, foams, gels, ointments, pads, pastes, pillows, powders, strands, or other formulations. In certain embodiment, fillers are non-adherent and may include a time-released antimicrobial. Exemplary fillers may be useful to maintain a moist environment, manage exudate, and for treatment of for example, partial- and full-thickness wounds, infected wounds, draining wounds, and deep wounds that require packing.

### Wound Treatment

General Aspects

The present invention is directed to pharmaceutical compositions and their methods of use wherein the composition comprises therapeutically effective amounts of one or more anti-cadherin agents alone or in combination with one or more therapeutic agents. The compositions are useful, for example, in enhancing or promoting healing of wounds, for example, including acute wounds and wounds that do not heal at expected rates, such as chronic wounds and other wounds that may be slow to heal or refractory to conventional wound treatment or wound healing promoting therapies, and other diseases, disorders and conditions described herein, including diseases, disorders and conditions characterized by inflammation or unwanted inflammation. Chronic wounds are often characterized by unwanted inflammation.

Equally, in instances of other tissue damage (particularly wounds) the methods and compositions of the invention are effective in promoting the wound healing process, reducing swelling and inflammation, and in minimizing scar formation. These formations are useful in treating fibrotic diseases, disorders and conditions and in treating, reducing the incidence or severity of or preventing or retarding adhesions, surgical adhesions and/or secondary surgical adhesions. The formulations have clear benefit in the treatment of wounds, whether the result of external trauma (including burns), internal trauma, or surgical intervention, as well as chronic wounds.

Compositions

Accordingly, in one aspect, the invention provides compositions for use in therapeutic wound treatment, which comprises an anti-cadherin agent and/or an anti_ZO-1 agent. In another aspect, the invention provides compositions for use in therapeutic wound treatment, which comprises at least one species of anti-cadherin agent and at least one other therapeutic agent, for example, an anti-connexin agent and/or an anti_ZO-1 agent. In a preferred embodiment, the compositions further comprise a pharmaceutically acceptable carrier or vehicle.

In one embodiment, the anti-cadherin agent is selected from a group consisting of: an anti-cadherin polynucleotide, an anti-cadherin peptide or peptidomimetic, an adherens junction modulator, and a cadherin complex modulator for wound treatment. In another embodiment, the anti-ZO-1 agent is selected from a group consisting of: an anti-ZO-1 polynucleotide, an anti-ZO-1 peptide or peptidomimetic, an adherens junction modulator, and a ZO-1 complex modulator for wound treatment.

In preferred embodiments, the anti-cadherin or anti-ZO-1 polynucleotide is an antisense polynucleotide. In one preferred form, the composition contains one or more antisense polynucleotides to the mRNA of one cadherin protein or one ZO-1 protein only. Most preferably, this cadherin protein is N-cadherin. In another preferred form, the composition comprises an anti-cadherin peptide or peptidomimetic and an antisense polynucleotide to the mRNA of a cadherin or ZO-1 protein. Most preferably, this cadherin is N-cadherin.

The compositions may comprise polynucleotides or anti-cadherin peptides or peptidomimetics, or other anti-cadherin agents with either or both, that are directed to more than one cadherin protein. The compositions may comprise polynucleotides or ZO-1 peptides or peptidomimetics, or other anti-ZO-1 agents with either or both, that are directed to more than one ZO-1 protein. One of the cadherin proteins to which polynucleotides or anti-cadherin peptides or other anti-cadherin agents are directed is N-cadherin. Other cadherins to which the polynucleotides or anti-cadherin peptides or other anti-cadherin agents are directed may include, for example, E-cadherin, P-cadherin, cadherin 11, cadherin 12, a protocadherin protein, a desmoglein protein, and a desmocollin protein. Suitable exemplary polynucleotides (and ODNs) directed to various connexins are set forth in Table_herein. Suitable anti-cadherin peptides are also provided herein. Suitable adherens junction or cadherin complex modulating agents are also described.

Accordingly, in one aspect, the invention provides compositions for use in treating wounds, including chronic and slow or delayed healing wounds. In another aspect, the invention provides compositions for use in treating fibrosis or fibrotic diseases, disorders, or conditions. In an alternate aspect, the invention provides compositions for use in preventing and/or treating abnormal or excessive scarring and/or excessive tissue proliferation and related disorders and conditions. In a further aspect, the invention provides compositions and methods for their use in preventing and/or decreasing adhesions, including surgical adhesions. In a further aspect, the invention provides compositions and methods for their use in preventing and/or decreasing inflammation.

Kits, Medicaments and Articles of Manufacture

Optionally, one or more anti-cadherin agents and/or anti-ZO-1 agents, either alone or in combination with one or more other therapeutic agents, may also be used in the manufacture of the medicament.

In one aspect, the invention provides a kit comprising one or more compositions or formulations described. For example, the kit may include a composition comprising an effective amount of one or more anti-cadherin agents and/or anti-ZO-1 agents, either alone or in combination with one or more other anti-cadherin agent species and/or anti-ZO-1 agents and/or other therapeutic agents.

Articles of manufacture are also provided, comprising a vessel containing a composition or formulation of the invention as described herein and instructions for use for the treatment of a subject. For example, in another aspect, the invention includes an article of manufacture comprising a vessel containing a therapeutically effective amount of one or more anti-cadherin agents, either alone or in combination with one or more other therapeutic agents, and instructions for use, including use for the treatment of a subject.

In one aspect, the invention provides for a kit for treating wounds, including chronic and slow or delayed healing wounds. In another aspect, the invention provides a kit for treating fibrosis or fibrotic diseases, disorders, or conditions. According to an alternate aspect, the invention provides a kit for preventing and/or treating abnormal or excessive scarring and/or excessive tissue proliferation and conditions comprising one or more of the formulations described. In another aspect, the invention provides a kit for preventing and/or decreasing adhesions comprising one or more compositions or formulations described. In another aspect, the invention provides a kit for preventing and/or decreasing inflammation comprising one or more compositions or formulations described.

Articles of manufacture are provided for preventing and/or treating wounds, including chronic and slow or delayed healing wounds. In another aspect, articles of manufacture are provided for preventing and/or treating fibrosis or fibrotic diseases, disorders, or conditions. Articles of manufacture are also provided for preventing and/or treating abnormal or excessive scarring and/or excessive tissue proliferation and related disorders and conditions. Additional articles of manufacture are provided for preventing and/or decreasing adhesions as described herein. Additional articles of manufacture are provided for preventing and/or decreasing inflammation as described herein.

Treatment

The compositions and formulations of the invention may be used in conjunction or combination with a composition for promoting the healing of wounds, for example, and can also be used to reduce swelling, inflammation, and/or scarring. The compositions and formulations of the invention may also be used in conjunction or combination with a composition for promoting and/or improving the healing of acute or chronic wounds, including slow-healing and delayed healing wounds. In one aspect, the wound will be the result of surgery or trauma or underlying medical condition, e.g., diabetes, peripheral edema, vasculitis, or cardiovascular disease.

In one aspect the invention is directed to a method of promoting or improving wound healing in a subject, comprising administration a therapeutically effective amount of one or more anti-cadherin agents, either alone or in combination with one or more other therapeutic agents. In certain embodiments, such administration is effective to reduce inflammation, promote cell migration to accelerate wound closure and healing, and/or to facilitate epithelial growth and surface recovery. In certain embodiments, the administration of one or more compositions of the invention is effective to reduce or prevent scar formation, including abnormal scar formation.

In one aspect the invention is directed to a method of promoting or improving wound healing in a subject, comprising administration of one or more anti-cadherin agents and/or anti-ZO-1 agents in combination with one or more other therapeutic agents in an amount effective to regulate adherens junction and/or cadherin complex formation and/or stability at wound or other sites of tissue injury or insult.

In yet a further aspect, the invention provides a method of decreasing scar formation and/or improving scar appearance in a patient who has suffered a wound, e.g., a surgical wound (such as in, for example, cosmetic, scar revision, and other surgeries), which comprises the step of administering one or more anti-cadherin agents and/or anti-ZO-1 agents, either alone or in combination with one or more other therapeutic agents, to said wound to downregulate expression of one or more cadherin protein(s) and/or ZO-1 protein(s) at and immediately adjacent the site of said wound. Again, the wound may be the result of trauma or surgery, for example, with the formulation being applied to the wound immediately prior to surgical repair and/or closure thereof. As noted herein, in methods to reduce or improve scar formation or appearance, or prevent or reduce inflammation, the anti-cadherin agent and/or anti-ZO-1 agent is preferably administered in combination with, or after or prior to, administration of a suitable amount of another wound healing agent, for example, an anti-connexin agent or anti-osteopontin agent.

In one aspect the invention is directed to a method of reducing, preventing, or ameliorating tissue damage (including inflammation damage) in a subject, comprising administration of one or more anti-cadherin agents and/or anti-ZO-1 agents, either alone or in combination with one or more other therapeutic agents.

In a further aspect, the invention is directed to a method of reducing swelling and/or inflammation, for example as part of treating an acute or chronic wound and/or tissue (including tissue subjected to physical trauma) which comprises the step of administering one or more anti-cadherin agents and/or anti-ZO-1 agents, either alone or in combination with one or more other therapeutic agents, to or proximate to said wound or tissue. In one embodiment the wound is the result of physical trauma to tissue, including dermal tissue (leading, for example, to a pressure ulcer) and neuronal tissue such as the brain, spinal cord, or optic nerve, or skin or eye.

In one aspect the invention is directed to sustained administration of one or more anti-cadherin agents and/or anti-ZO-1 agents, either alone or in combination with one or more other therapeutic agents. In one embodiment, the agent or agents are administered for at least at least about 0.5 hours, about 1-24 hours, at least about 2, hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours or at least about 24 hours. In one embodiment, cadherin expression is downregulated over a sustained period of time. In another embodiment, cadherin complex and/or adherens junction formation and/or stability is modulated, preferably inhibited, partially or completely, over a preferred period of time. Preferably, such period of time is at least about 1, 2, 4, 6, 8, 10, 12, or 24 hours. According to one embodiment, the wound is a chronic wound. Suitable subjects include a diabetic subject. Other subjects include, for example, those with peripheral edema, vasculitis, or cardiovascular disease. Other subjects include, for example, those with venous disease, including venous insufficiency, or arterial disease, including arterial insufficiency.

In one aspect, the present invention provides a method of treating a subject having a wound that comprises sustained administration of an effective amount of one or more anti-cadherin agents and/or anti-ZO-1 agents, either alone or in combination with one or more other therapeutic agents, to the wound.

In another aspect, methods for treating a subject having a chronic wound are provided. Such methods include administering to the subject one or more anti-cadherin agents and/or anti-ZO-1 agents, either alone or in combination with one or more other therapeutic agents.

In one aspect the invention is directed to a method for treatment or prophylaxis of a chronic wound comprising administering to a subject in need thereof an effective amount of an anti-cadherin agent and/or an anti-ZO-1 agent, either alone or in combination with one or more other therapeutic agents. In one embodiment, the chronic wound is a chronic skin wound and a composition of the present invention is administered to the skin or a tissue associated with the skin of said subject for an effective period of time. A chronic skin wound suitable for treatment may, for example, be selected from the group consisting of pressure ulcers, diabetic ulcers, venous ulcers, arterial ulcers, vasculitic ulcers, and mixed ulcers, and other noted herein. The chronic wound may be an arterial ulcer, which comprises ulcerations resulting from complete or partial arterial blockage. The chronic wound may be a venous stasis ulcer, which comprises ulcerations resulting from a malfunction of the venous valve and the associated vascular disease. The chronic wound may be a trauma-induced ulcer. The chronic, slow- or delayed-healing wound may be, for example, dermal or ocular, associated with another organ tissue (e.g., kidney, bowel, liver, lung), or in the CNS.

When not administered as a fixed combination, preferred combination therapy methods include the sequential administration of one or more anti-cadherin agents and/or anti-ZO-1 agents and one or more other therapeutic agents. Preferably, the agents are administered sequentially within at least about one-half hour of each other. The agents may also be administered with about one hour of each other, with about one day to about one week of each other, or as otherwise deemed appropriate.

In one embodiment the method for treatment or prophylaxis of a chronic wound comprises sustained administration of one or more anti-cadherin agents and/or anti-ZO-1 agents, either alone or in combination with one or more other therapeutic agents. In one embodiment, the composition or compositions are administered in a sustained release formulation. In another embodiment, the composition or compositions are administered for a sustained period of time. Conveniently, the composition is effective to decrease cadherin and/or ZO-1 protein levels, or block or reduce cadherin or ZO-1 complexes and or adherens junction formation and/or stability, for at least about 1-2 hours, about 2-4 hours, about 4-6 hours, about 4-8 hours, about 12 hours, about 18 hours, or about 24 hours. Subjects that may be treated include diabetic subjects, and patients with other ulcers, including venous ulcers and others described herein and known in the art.

In one aspect the invention is directed to a method of preventing and/or treating fibrosis or fibrotic diseases, disorders or conditions in a subject, comprising administration a therapeutically effective amount of a composition according to the invention. In certain embodiments, the administration is effective to reduce fibrosis. In certain embodiments, the administration is effective to prevent or reduce contracture.

In one aspect the invention is directed to a method of preventing and/or treating fibrosis or fibrotic diseases, disorders, or conditions in a subject, comprising administration of a therapeutically effective amount of one or more anti-cadherin agents and/or anti-ZO-1 agents effective to reduce fibrosis. In one embodiment, administration of the anti-cadherin agent and/or anti-ZO-1 agent and, optionally, one or more anti-connexin agents, is effective to prevent or reduce contracture.

According to one embodiment of the method, the subject has a disorder selected from the group consisting of scleroderma, kidney fibrosis (including diabetic nephropathy), cardiac fibrosis (e.g. myocardial fibrosis), pulmonary fibrosis (e.g., glomerulosclerosis pulmonary fibrosis, idiopathic pulmonary fibrosis, silicosis, asbestosis, interstitial lung disease and fibrotic lung disease, and chemotherapy/radiation induced pulmonary fibrosis), oral fibrosis, endomyocardial fibrosis, deltoid fibrosis, pancreatitis, inflammatory bowel disease, Crohn's disease, nodular fascilitis, eosinophilic fasciitis, general fibrosis syndrome characterized by replacement of normal muscle tissue by fibrous tissue in varying degrees, retroperitoneal fibrosis, liver fibrosis, liver cirrhosis, chronic renal failure; myelofibrosis (bone marrow fibrosis), drug induced ergotism, glioblastoma in Li-Fraumeni syndrome, sporadic glioblastoma, myleoid leukemia, acute myelogenous leukemia, myelodysplastic syndrome, myeloproferative syndrome, gynecological cancer, Kaposi's sarcoma, Hansen's disease, collagenous colitis and acute fibrosis. According to this embodiment, the scleroderma may be morphea, generalized morphea, or linear scleroderma. Also according to this embodiment, the kidney fibrosis may be glomerular sclerosis, renal tubulointerstitial fibrosis or progressive renal disease. Further to this embodiment, the pulmonary fibrosis may be diffuse interstitial pulmonary fibrosis.

According to another embodiment of the method, the fibrosis is acute fibrosis. The acute fibrosis may be in response to various forms of trauma including accidental injuries, infections, radiation or chemotherapy treatments.

According to another embodiment of the method, the fibrosis is chronic fibrosis. The invention also includes methods for treating and/or preventing, in whole or in part, various diseases, disorders and conditions, including, for example, capsular contracture, Dupytren's contracture, Volkmann's contracture, Ledderhose's contracture, Peyronie's contracture or recurrence thereof, comprising administering a effective amount of a composition comprising an anti-cadherin agent, preferably an anti-cadherin polynucleotide. In some preferred embodiments, the composition is administered to the site of the injury before, at the time of and/or after a release procedure (e.g., forced manipulation, open release, arthroscopic release, or debulking of scar) to prevent the recurrence of scarred and abnormal tissue and/or further contracture.

In one aspect the invention is directed to a method of for preventing and/or treating abnormal or excessive scarring and/or excessive tissue proliferation and related disorders and conditions in a subject, comprising administration a therapeutically effective amount of one or more one or more anti-cadherin agents and/or anti-ZO-1 agents, either alone or in combination with one or more other therapeutic agents. In certain embodiments, the administration is effective to reduce abnormal or excessive scarring and/or excessive tissue proliferation and related disorders and conditions.

In one aspect the invention is directed to a method of for preventing and/or treating abnormal or excessive scarring and/or excessive tissue proliferation and related disorders and conditions in a subject, comprising administration a therapeutically effective amount of an anti-cadherin and/or anti-ZO-1 agent. In one embodiment, the anti-cadherin and/or anti-ZO-1 agent is effective to reduce abnormal or excessive scarring and/or excessive tissue proliferation and related disorders and conditions.

In one aspect the invention is directed to sustained administration of an anti-cadherin and/or anti-ZO-1 agent alone or in combination with one or more other therapeutic agents.

According to one embodiment, the subject has an abnormal scar selected from the group consisting of keloid scars, hypertrophic scars, widespread scars, and atrophic scars.

According to another embodiment, the subjects to be treated include those having experienced trauma, surgical intervention, burns, and other types of injuries that lead, or can lead, to abnormal or excessive scarring, as well as excessive scar formation and other types of abnormal proliferation of tissue, including keloid scars, hypertrophic scars, widespread scars, and atrophic scars.

In certain embodiments, the anti-cadherin and/or anti-ZO-1 agent(s), alone or in combination with one or more other therapeutic agents is administered to epithelial, connective, muscle, and nerve tissue or other tissue exposed or wounded during surgery or as a result of trauma. In some embodiments, the anti-cadherin agent is administered topically. In other embodiments, the anti-cadherin and/or anti-ZO-1 agent is implanted or instilled or injected.

The following examples which will be understood to be provided by way of illustration only and not to constitute a limitation on the scope of the invention.

### EXAMPLES

### Example 1

### Oligonucleotides Targeting N-Cadherin

This example describes several candidates for N-cadherin AS ODNs (antisense oligodeoxtribonucleotides) and shRNAs (small hairpin RNA molecules).

The nucleotide sequence of human N-cadherin is known. The nucleotide sequence of full-length N-cadherin cDNA (Genbank accession number EL733845; SEQ ID NO:2) comprises 2,721 nucleotide bases and encodes 906 amino acids, and is as follows:

| | |
|---|---|
| atgtgccgga tagcgggagc gctgcggacc ctgctgccgc tgctggcggc cctgcttcag gcgtctgtag | 70 |
| aggcttctgg tgaaatcgca ttatgcaaga ctggatttcc tgaagatgtt tacagtgcag tcttatcgaa | 140 |
| ggatgtgcat gaaggacagc ctcttctcaa tgtgaagttt agcaactgca atggaaaaag aaaagtacaa | 210 |
| tatgagagca gtgagcctgc agattttaag gtggatgaag atggcatggt gtatgccgtg agaagctttc | 280 |
| cactctcttc tgaacatgcc aagttcctga tatatgccca agacaaagag acccaggaaa agtggcaagt | 350 |
| ggcagtaaaa ttgagcctga agccaacctt aactgaggag tcagtgaagg agtcagcaga agttgaagaa | 420 |
| atagtgttcc caagacaatt cagtaagcac agtggccacc tacaaaggca gaagagagac tgggtcatct | 490 |
| ctccaatcaa cttgccagaa aactccaggg gaccttttcc tcaagagctt gtcaggatca ggtctgatag | 560 |
| agataaaaac ctttcactgc ggtacagtgt aactgggcca ggagctgacc agcctccaac tggtatcttc | 630 |
| attatcaacc ccatctcggg tcagctgtcg gtgacaaagc ccctggatcg cgagcagata gcccggtttc | 700 |
| atttgagggc acatgcagta gatattaatg gaaatcaagt ggagaacccc attgacattg tcatcaatgt | 770 |
| tattgacatg aatgacaaca gacctgagtt cttacaccag gtttggaatg ggacagttcc tgagggatca | 840 |
| aagcctggaa catatgtgat gaccgtaaca gcaattgatg ctgacgatcc caatgccctc aatgggatgt | 910 |
| tgaggtacag aatcgtgtct caggctccaa gcaccccttc acccaacatg tttacaatca acaatgagac | 980 |
| tggtgacatc atcacagtgg cagctggact tgatcgagaa aaagtgcaac agtatacgtt aataattcaa | 1050 |
| gctacagaca tggaaggcaa tcccacatat ggcctttcaa acacagccac ggccgtcatc acagtgacag | 1120 |
| atgtcaatga caatcctcca gagtttactg ccatgacgtt ttatggtgaa gttcctgaga acagggtaga | 1190 |
| catcatagta gctaatctaa ctgtgaccga taaggatcaa ccccatacac cagcctggaa cgcagtgtac | 1260 |
| agaatcagtg gcggagatcc tactggacgg ttcgccatcc agaccgacca aaacagcaac gacgggttag | 1330 |
| tcaccgtggt caaaccaatc gactttgaag caaataggat gtttgtcctt actgttgctg cagaaaatca | 1400 |
| agtgccatta gccaagggaa ttcagcaccc gcctcagtca actgcaacca tgtctgttac agttattgac | 1470 |
| gtaaatgaaa acccttattt tgcccccaat cctaagatca ttcgccaaga agaagggctt catgccggta | 1540 |
| ccatgttgac aacattcact gctcaggacc cagatcgata tatgcagcaa aatattagat acactaaatt | 1610 |
| atctgatcct gccaattggc taaaaataga tcctgtgaat ggacaaataa ctacaattgc tgttttggac | 1680 |
| cgagaatcac caaatgtgaa aaacaatata tataatgcta ctttccttgc ttctgacaat ggaattcctc | 1750 |
| ctatgagtgg aacaggaacg ctgcagatct atttacttga tattaatgac aatgcccctc aagtgttacc | 1820 |
| tcaagaggca gagacttgcg aaactccaga ccccaattca attaatatta cagcacttga ttatggcatt | 1890 |
| gatccaaatg ctggaccatt tgcttttgat cttcctttat ctccagtgac tattaagaga aattggacca | 1960 |
| tcactcggct taatggtgat tttgctcagc ttaatttaaa gataaaattt cttgaagctg gtatctatga | 2030 |
| agttcccatc ataatcacag attcgggtaa tcctcccaaa tcaaatattt ccatactgcg cgtgaaggtt | 2100 |
| tgccagtgtg actccaacgg ggactgcaca gatgtggaca ggattgtggg tgcggggctt ggcaccggtg | 2170 |
| ccatcattgc catcctgctc tgcatcatca tcctgcttat ccttgtgctg atgtttgtgg tatggatgaa | 2240 |
| acgccgggat aaagaacgcc aggccaaaca acttttaatt gatccagaag atgatgtaag agataatatt | 2310 |
| ttaaaatatg atgaagaagg tggaggagaa gaagaccagg actatgactt gagccagctg cagcagcctg | 2380 |
| acactgtgga gcctgatgcc atcaagcctg tgggaatccg acgaatggat gaaagaccca tccacgctga | 2450 |
| gccccagtat ccggtccgat ctgcagcccc acaccctgga gacattgggg acttcattaa tgagggcctt | 2520 |
| aaagcggctg acaatgaccc cacagctcca ccatatgact ccctgttagt gtttgactat gaaggcagtg | 2590 |
| gctccactgc tgggtccttg agctccctta attcctcaag tagtggtggt gagcaggact atgattacct | 2660 |
| gaacgactgg gggccacggt tcaagaaactt gctgacatg tatggtggag gtgatgactg a | 2721 |

Any antisense molecule or shRNA that targets a region suitable to disrupt N-cadherin expression will be useful in the practicing the invention. One suitable approach in this regard concerns RNA interference (RNAi). As is the case with the expression of many other genes, N-cadherin expression can be knocked down in vivo or vitro through the use RNAi. A representative class of molecules that can be used for RNAi are short hairpin RNAs (shRNAs). One such anti-N-cadherin shRNA molecule can be constructed as follows using the vector termed pSuper-Ncad. This vector is assembled using the following sense and antisense oligonucleotides (ONs):

Sense ODN:
(SEQ ID NO: 3)
5′-GATCCCCGATGTTTACAGCGCAGTCTTTCAAGAGAAGACTGCGCTGT
AAACATCTTTTTGGAAA-3′
Antisense ON:
(SEQ ID NO: 4)
5′-AGCTTTTCCAAAAAGATGTTTACAGCGCAGTCTCTTGAAAGACTGCG
CTGTAAACATCGGG-3′

The sense and antisense oligos can be annealed and ligated into a linealized pSuper vector (OligoEngine: catalog VEC-PBS-0011). This target sequence corresponds to that of RNAi against rat Ncad reported by Fairless, et al. ((2005), Mol. Cell. Neurosci., vol. 28:253-263). As a negative control, an shRNA vector consisting of scrambled target sequence of Ncad can be designed to construct a negative control. As an example, the following sense and antisense ONs can be used to construct a vector termed pSuper-Ncad-scrambled. The sense and antisense sequences are:

Sense ON:
(SEQ ID NO: 5)
5′-GATCCCCGCTATCGCTACGTGTAAGTTTCAAGAGAACTTACACGTAG
CGATAGCTTTTTGGAAA-3′
Antisense ON:
(SEQ ID NO: 6)
5′-AGCTTTTCCAAAAAGCTATCGCTACGTGTAAGTTCTCTTGAAACTTA
CACGTAGCGATAGCGGG-3′

Antisense and short hairpin RNAs targeted against human N-cadherin also include those that target specific sequences. One such representative target nucleotide sequence in the N-cadherin gene is: 5′-GACTGGATTTCCTGAAGAT-3′ (SEQ ID NO:7); nucleotides 215-233 of human N-cadherin GenBank accession number BCO36470, which are also bases 99-117 of GenBank accession number EL733845; SEQ ID NO:8, above).

### Example 2

### Targeting Cx43 and N-Cadherin, which are Abnormally Up-Regulated in Venous Leg Ulcers, Influences Migration, Adhesion, and Activation of Rho GTPases

### Overview

Venous leg ulcers can be very hard to heal and represent a significant medical need with no effective therapeutic treatment currently available. In the experiments described in this Example, in wound edge biopsies from human venous leg ulcers it was found a striking up-regulation of dermal N-cadherin (N-cad), Zonular Occludens-1 (ZO-1), and the gap junction protein Connexin43 (Cx43) compared to these proteins levels observed in intact skin, and in stark contrast to the down-regulation of Cx43 expression seen in acute, healing wounds. The expression of these proteins was targeted in 3T3 fibroblasts to evaluate their roles in venous leg ulcers healing. Knockdown of Cx43 and N-cad accelerated cell migration in a scratch wound-healing assay. Reducing Cx43 increased Golgi reorientation, while decreasing cell adhesion and proliferation. Furthermore, Connexin43 and N-cad knockdown led to profound effects on fibroblast cytoskeletal dynamics after scratch-wounding. The cells exhibited longer lamelipodial protrusions lacking the F-actin belt seen at the leading edge in wounded control cells. This phenotype was accompanied by augmented activation of Rac-1 and RhoA GTPases, as revealed by Forster Resonance Energy Transfer and pull down experiments. Amongst other things, these results show that Cx43 and N-cad will be therapeutic targets in promoting healing of venous leg ulcers, by acting at least in part through distinct contributions of cell adhesion, migration, proliferation, and cytoskeletal dynamics.

### Introduction

Chronic wounds, such as diabetic foot ulcers, pressure ulcers, and venous leg ulcers (VLU), are an increasing worldwide problem, with estimates that 1-2% of the population in Western countries will develop a chronic wound over the course of their lifetimes. Chronic wounds represent a major economic burden on healthcare services, with an estimated annual USA expenditure of $25 billion. With growing numbers of elderly and diabetics in the population, this expenditure figure is expected to rise in coming years. Unfortunately, there are few effective therapeutic options for these debilitating wounds, and there remains a significant need for effective new treatments.

Cx43 is the most ubiquitous connexin protein in the skin, expressed in keratinocytes, fibroblasts, endothelial cells, and dermal appendages. It is known that the topical application of a Cx43-specific antisense-containing gel to acute wounds in rodent models significantly accelerates the healing process while reducing inflammation and scar size.

In the normal healing process Cx43 protein becomes down-regulated in keratinocytes in the first 24-48 hours as the cells become migratory and crawl forward to close the wound. Following experiments in Cx43 conditional knockout mice it was later reported that down-regulation of Cx43 appears to be a prerequisite for the coordinated proliferation and mobilization of keratinocytes during wound healing. In contrast, it was shown that in STZ diabetic rats, a model for chronic wounds, Cx43 is up-regulated in wound edge keratinocytes instead of being down-regulated, and that migration is delayed until down-regulation occurs. Application of a Cx43 antisense to STZ diabetic rat wounds prevented the abnormal up-regulation of Cx43 and restored wound closure to normal rates or better. Over-expression of Cx43 was also shown to inhibit corneal endothelial wound healing in an in vivo rat corneal scrape injury model, while knockdown with Cx43 antisense sped it up. Cx43 was also detected in the cells at the wound margins of the majority of biopsies taken from nine mixed and two diabetic leg ulcers.

One of the key impediments to the healing of chronic wounds is the failure of fibroblasts to migrate, proliferate, and generate granulation tissue. Most previous reports have concentrated on epidermal Cx43 in wound healing, and little attention has been paid to Cx43 in dermal fibroblasts. In the work described in this Example, a combination of in vivo and in vitro models were used to analyze the implications of elevated Cx43 expression, which was discovered to be detrimentally up-regulated in the dermis of human chronic VLU, and to correlate with reduced rates of migration of scratch-wounded fibroblasts over-expressing Cx43. In addition to Cx43, it was also discovered that ZO-1 and N-cad, which interact with Cx43 and each other, are abnormally over-expressed in the dermis of human chronic VLU. Targeting Cx43 reduced the expression levels of ZO-1 and N-cad both in vitro and in vivo. Knock down of Cx43 and N-cad, but not ZO-1 alone, accelerated cell migration in a scratch wound-healing assay. Reduction of Cx43 or N-cad also increased Golgi polarization while reducing proliferation and cell adhesion in fibroblasts. Targeting of Cx43 or N-cad, furthermore, was accompanied by cytoskeletal changes, increased lamellipodia protrusion, and activation of Rho GTPases. These results support therapeutically targeting Cx43 and N-cad to improve wound repair through a mechanism involving remodeling of cell contacts and adhesion-dependent cytoskeletal modifications in fibroblasts.

### Materials and Methods

### Human Chronic VLU and Matched Intact Skin Samples

Collection of wound edge punch biopsies from chronic venous ulcer leg wounds and normal arm biopsies was performed after obtaining written informed consent and in accordance with applicable guidelines. Briefly, for the VLUs a single 4 mm punch biopsy was taken from the wound edge under local anesthesia in patients with clinically confirmed venous ulceration. A second 4 mm punch biopsy was taken from normal skin on the forearm in the same subjects. In all, biopsies from 6 subjects with clinically confirmed VLU (three male, three female; range 38-79 years) were taken. The median ulcer duration was 6 months (range 1.5-36 months), and the median size was 10 cm² (range 2-113 cm²). The VLU biopsies were initially fixed overnight with 4% paraformaldehyde (PFA), then transferred to 25% sucrose, and stored at 4° C. until processing. For immunohistochemistry, tissue biopsies were embedded in OCT (BDH, UK). Samples were cryo-sectioned (10 μm) for immunohistochemistry analysis. For the normal (acute) wound biopsies, an initial 6 mm punch biopsy was performed under local anesthetic on the anterolateral thigh of three healthy male volunteers (range 20-36 years) and then 4 hours later the punched wound site was excised with a larger 10 mm punch, and the resulting wound sutured closed. The 2 mm wide donut shaped biopsy was immediately imbedded in OCT, snap frozen in liquid nitrogen, and then stored at −80° C. until being cryo-sectioned before analysis.

### Mouse and Rat Cutaneous Wound-Healing Model and ODNs Application

Male, 8-week-old ICR mice or Sprague-Dawley rats from UCL's Biological Services Unit were maintained according to UK Home Office animal regulations. Excisional lesions were performed as previously described (Mori, et al. (2006), J Cell Sci, vol. 119: 5193-5203). Single topical applications of 10 μM unmodified Cx43asODN and control Cx43sODN (Sigma-Genosys) were delivered to each of two independent wounds in 30% Pluronic F-127 gel (Sigma). Two days after wounding, animals were humanely sacrificed and the wound tissue was harvested.

### Cell Cultures and ODNs Treatment

3T3 fibroblasts were grown in DMEM-GlutaMAX™-1 (Gibco, Invitrogen) supplemented with 10% FBS (Gibco, Invitrogen) in 5% CO₂ at 37° C. For ODN treatments fibroblasts were washed with PBS to eliminate any traces of serum, and were incubated in serum free media for 2 hours with 20 μM Cx43asODN or Cx43sODN (Qiu, et al., (2003), Curr Biol, vol. 13: 1697-1703) (Sigma-Genosys) or with 10 μM ZO-1asODN or ZO-lsODNs (Underwood, et al. (1999), Am J Physiol, vol. 277: C330-342) (Sigma-Genosys). Following this incubation, the medium with the ODNs was removed and replaced by 10% FBS in DMEM.

### Transfection; Retroviral and Lentiviral Constructs and Transduction

A retroviral pSuper vector containing the Cx43-specific shRNA target sequence 5′-GGTGTGGCTGTCAGTGCTC-3′ (SEQ ID NO:9; van Zeijl, et al. (2007), J Cell Biol, vol. 177: 881-891), here, designated Cx43shRNA, was used to establish a stable knockdown of Cx43 in 3T3 fibroblasts. A pSuppressor (p.Sup) retroviral vector (Imgenex Co) was used as a control. The packaging cell line GP2-293 (Clontech) was transfected with the p.Sup and Cx43shRNA constructs as previously described (Carr and Whitmore (2005), Nat Cell Biol, vol. 7: 319-321). Mock and N-cad shRNA constructs (Hosokawa, et al. (2010), Blood, vol. 116: 554-563), were transfected into HEK 293T cells as described (Demaison, et al. (2002), Hum Gene Ther, vol. 13: 803-813). 3T3 fibroblasts were transduced with retrovirus or lentivirus for 2 days, and Cx43shRNA and N-cadshRNA and Mock-transduced cells were selected on the basis of resistance to 2 μg/ml puromycin or 500 μg/ml of geneticin (for p.Sup).

Fugene HD (Roche) was used to transfect 70% confluent fibroblasts with 1 mg/ml pGFP, Cx43-DN, or Cx43-WT constructs as described (Becker, et al. (2001), Cell Commun Adhes, vol. 8: 355-359). Media was changed the following morning and 24 h later cells were used in different sets of experiments.

### Immunohistochemistry of Murine and Human Skin Samples

Immunostaining was carried out on 6-nm cryostat sections of wounded rat and mouse skin, or 10-μm human chronic VLU or matched non-wounded skin, fixed in acetone at 4° C. for 5 minutes. Primary antibodies (Abs) for Cx43 (diluted 1:2000; Sigma, 6219), N-cadherin (diluted 1:100; Abcam, ab18203), and ZO-1 (diluted 1:100; Zymed Laboratories, 61-7300) were incubated for 1 hour at room temperature. Sections were washed with PBS and then incubated with the respective secondary antibodies, swine anti-rabbit FITC-conjugated (DAKO) and goat anti-mouse Cy3-conjugated (Pierce). Secondary antibody incubation in the absence of primary antibodies was used as a negative control. Sections were then counterstained for 10 minutes with 5 μg/ml of the nuclear dye Hoechst 33342 (Sigma) and single optical sections were acquired on a Leica SP2 confocal microscope (Leica Microsystems, UK). All parameters during image acquisition were kept constant throughout each experiment to allow direct comparison of all of the 8 bit digital images Immunostaining levels were quantified per unit area using a well-established pixel-counting method (Wang, et al. (2007), Diabetes, vol. 56: 2809-2817) using ImageJ software (NIH). Images were converted to binary images using an identical threshold. Objects greater than two pixels were counted in order to generate a readout of the number of positive pixels per unit area for comparison between conditions.

### Immunostaining

Confluent fibroblast monolayers were wounded and fixed 3 h later with 4% PFA for 10 minutes and permeabilized with 0.1% Triton X-100. Primary antibodies for Cx43 (diluted 1:2000; Sigma), ZO-1 (diluted 1:100; Zymed Laboratories), N-cadherin (diluted 1:100; Abcam), β-catenin (diluted 1:200; ab6302 Abcam), α-catenin (diluted 1:200; C2081 Sigma), tyrosinated tubulin (diluted 1:400; YL1/2; Ab6160 Abcam), and acetylated tubulin (diluted 1:200; T7451 Sigma) were used according to the manufacturer's recommendations. Incubation with the appropriate secondary antibodies (Alexa 488 or 633; Molecular Probes) was followed by 10 minutes application of 5 μg/ml of the nuclear dye Hoechst 33342 (Sigma). Secondary antibody incubation in the absence of primary antibody was used as negative control. TRITC-phalloidin (Sigma) was included in some experiments for visualization of F-actin. Cells were imaged using a 63×, 1.25 NA objective on a Leica SP2 confocal microscope (Leica Microsystems, UK). All acquisition parameters were kept constant throughout each experiment and staining was quantified based on pixel counts (Wang, et al. (2007), above).

### Golgi Reorientation Measurements

Measurement of Golgi reorientation was performed as described previously (Magdalena, et al. (2003), J Cell Sci, vol. 116: 743-756). Confluent fibroblasts were scratched and incubated for 3 h. After this time, cells were fixed with 4% PFA and stained with anti-GM130 (BD Biosciences) and Hoechst 33342 nuclear stain (Sigma). One hundred cells in ten randomly selected fields were evaluated for Golgi orientation.

### Cell Proliferation

3T3 fibroblasts (1×10⁴ cells) transduced with pSup, Cx43shRNA, Mock, and N-cadshRNA constructs were plated into 96-well plates and cell growth was monitored in real-time using the IncuCyte™ live-cell imaging system (Essen Instruments, Ann Arbor, Mich.). Experiments were performed at least three times in duplicate and data was expressed as percentage of confluence.

### Measurement of Protrusion Length

Confluent fibroblasts transduced with Cx43shRNA, p.Sup, N-cadshRNA, and Mock constructs, or transfected with pGFP, Cx43-DN, or Cx43-WT were wounded and fixed with 4% PFA 3 h later. The cells were then stained with Hoechst 33342 nuclear stain (Sigma). To quantify protrusion length, the distance from the nuclei to the leading edge of wound edge cells was measured. In each of three independent experiments, 3-5 transfected cells, or 30 transduced cells in randomly selected fields, were analyzed to calculate the average protrusion length.

### Western Blots

Cell pellets from harvested fibroblasts (Cx43shRNA- or p.Sup-transduced) were suspended at 4° C. in ice-cold RIPA buffer plus protease inhibitors. Equal amounts of protein were resuspended in Laemmli 4× sample buffer, separated by 10% SDS-PAGE and visualized with primary Abs to Cx43 (diluted 1:2000; Sigma), ZO-1 (diluted 1:500; Zymed Laboratories), α-catenin (diluted 1:200; Sigma), β-catenin β-catenin (diluted 1:1000; Abcam), or N-cadherin (diluted 1:500; Abcam). Antibodies against α-tubulin or actin (both from Sigma) were used as loading controls. Secondary antibodies were HRP-conjugated, and protein levels were visualized using an enhanced chemiluminescence (ECL) system (Amersham). The ratio protein/tubulin or/actin was determined by scanning and quantifying the bands, using ImageJ software (NIH).

### Dye-Transfer Assays

Dye-injection was carried out according to the method described previously (Becker, et al. (1995), J Cell Sci, vol. 108 (Pt 4): 1455-1467). Cells were impaled under visual control and filled with dye by iontophoresis. Communication was assessed 1 minute after cessation of iontophoresis. At least 10 injections were performed for each set of experimental conditions. The coupling was tested in cells: a) treated with Cx43sODN, Cx43asODN, or control untreated cells; b) transiently transfected with Cx43-DN, Cx43-WT, or pGFP; and c) Cx43shRNA or p.Sup-transduced cells. Images of microinjected cells were acquired with a 40×0.8 NA objective on a Leica DMLFS microscope (Leica Microsystems, UK) using Volocity acquisition software (Improvision/Perkin Elmer).

### Cell Migration Assays

Confluent cultures of Cx43sODN, Cx43asODN, ZO-1asODN, and ZO-1sODN-treated fibroblasts; or Cx43shRNA, p.Sup, N-cadshRNA, and Mock-transduced cells, as well as Cx43-WT, Cx43-DN, and pGFP-transfected fibroblasts were subjected to a mechanical scratch-wound. In some experiments, Cx43shRNA and p.Sup fibroblasts were serum starved (SS) or incubated in the presence of serum (FBS) for 48 h as described (Francis, et al. (2011), PloS one, vol. 6: e26379). After this period of time, cells were wounded and incubated in DMEM supplemented with serum. Time-lapse images were taken soon after wounding at intervals of 5 minutes for 3-4 hours, and were acquired on an inverted Zeiss LSM AxioPlan 400 fluorescence microscope (Zeiss, UK), equipped with a Orca CCD camera (Hamamatsu, UK), using a 40×1.2NA objective with an incubation chamber at 37° and 5% CO₂. Time-lapse images were captured using OpenLab image acquisition software (Improvision/Perkin Elmer, UK). The migration velocities of individual fibroblasts were quantified using Volocity4 analysis software (Improvision/Perkin Elmer) by measuring the distance between the initial and final positions of leading edge cells 3 hours after wounding. For each experimental condition, velocities were presented as mean±SD (μm/sec).

### Rho GTPase Activation Assays

Fresh lysates were used for Rac1/Cdc42 and RhoA-pulldown assays, using the Rac/Cdc42-binding domain (p21-binding domain, PBD) of PAK, as previously described (Mendoza-Naranjo, et al. (2007), J Cell Sci, vol. 120: 279-288.), or GST-Rhotekin to detect relative amounts of RhoA-GTP. Bound protein (GTP-bound Rac1, Cdc42, or RhoA) levels were detected by western blot. Total Rac1, Cdc42, and RhoA from total cell lysates were also analyzed as loading controls.

Förster Resonance Energy Transfer (FRET) Assays

Plasmids encoding FRET probes Raichu-Rac1, Raichu-Cdc42 (Itoh, et al. (2002), Mol Cell Biol, vol. 22: 6582-6591), and RhoA biosensor (Pertz, et al. (2006), Nature, vol. 440: 1069-1072) were transfected into Cx43shRNA or p.Sup-transduced fibroblasts using Fugene HD (Roche). The FRET efficiency of the cells expressing the EYFP-ECFP fusion construct was measured using confocal fluorescence microscopy and acceptor photobleaching. Leading-edge transfected cells were imaged using an Olympus FluoView 1000 laser scanning confocal microscope and a 60×1.4NA oil objective (Olympus Microscopes, UK). The CFP and YFP channels were excited using the 440 nm and 515 nm lasers, respectively. The two emission channels were 460-510 nm for CFP and 520-620 nm for YFP. The gain for each channel was set to approximately 75% of dynamic range (12-bit, 4096 grey levels) and offsets set such that backgrounds were zero. Pre- and post-bleach CFP and YFP images were acquired and the FRET mode was used to collect images for each channel after acceptor bleaching with 10-12 scans of the 515 nm argon laser line at maximum power (to bleach YFP). Olympus FluoView 1000 software was used to analyze donor and acceptor intensity values before (pre) and after (post) bleaching and values were then extracted from pixels falling inside the cell of interest, as well as an equal-sized bleached region outside the cell, and the mean ratio was determined for each region. The FRET efficiency ratio over the whole cell was calculated using the following formula: FRET efficiency=[ID₍ₚₒₛₜ₎−ID₍ₚᵣₑ₎]/ID₍ₚₒₛₜ₎, where IDₚᵣₑ and IDₚₒₛₜ refer to the intensity of the donor (CFP) before (pre) and after (post) photobleaching of the acceptor (YFP).

### Hanging Drop Assays

Approximately 20,000 single cells were suspended in 35 μl drops of 10% FBS in DMEM from the lids of 24-well dishes for 4 hours. Water was placed at the bottom of each well to maintain humidity. The drops were pipetted five times up and down with 200 μl yellow tips, fixed with 4% paraformaldehyde, and aliquots were spread on coverslips. Images of six random fields from six individual samples for each condition studied (p.Sup and Cx43shRNA-transduced cells) were taken with a 4×0.1 NA objective on an inverted Axiovert Zeiss LSM microscope. An area of approximately 2.5×10²-7.5×10² μm² corresponds to a cluster of 1-3 cells and 2.5×10³ μm² corresponds to about 10 cells. The area of each cell cluster was determined using a custom-written plug-in for ImageJ software (NIH). The area occupied by single cells was measured in parallel to estimate the amount of cells per unit area.

### Statistical Analysis

Statistical differences were determined using Wilcoxon Matched-Pairs Signed-Ranks Test for paired data, or one-way analysis of variance (ANOVA) for data sets of multiple comparisons. A two-tailed Chi-squared test was used for the analysis of cell cluster sizes in the hanging drop assay of adhesion. All data are presented as the mean±SD except where stated. Criterion levels for the individual tests are given in the Results, below.

### Results

### Cx43, N-Cadherin and ZO-1 are Dramatically Up-Regulated in the Dermis of Human VLU

Skin punch biopsies, 2 mm, from healthy human volunteers and chronic wound edge biopsy samples, 4 mm, from 6 human VLU, together with corresponding intact skin samples from the same patients, were used to analyze Cx43 protein levels in the dermis of chronic versus acute wounds. As shown in FIG. 1, dermal Cx43 proteins levels are greatly up-regulated in human chronic VLU. FIG. 1, D, is a representative picture of a chronic VLU in the lower leg of a patient, from which a wound edge punch biopsy has been taken (white dotted circle in photo, FIG. 1, D).

In acute wounds, down-regulation of Cx43 was seen at dermal wound margins 4 hours after excisional wounding (FIG. 1A; scale bar=25 μm). In the photo shown in FIG. 1A, blue signal is Hoechst staining of nuclei and collagen bundle autofluorescence, and the dotted white line shows the border between the epidermis and the dermis. These results were similar to those observed in the wound-edge dermis in a murine model after excisional wounding (FIG. 9, A; scale bar=25 μm), where the expression and distribution of Cx43 in mouse skin dermis was examined by immunohistochemistry two days after excisional wounding. In these mouse experiments, wound-edge Cx43 was reduced in dermal fibroblasts after such wounds. In FIG. 9, A, arrowheads show how Cx43 becomes more prevalent with increasing distance from the wound edge. Cx43 levels were quantified along the wound site and were significantly lower at the wound edge (p<0.005). Values in the graph shown in FIG. 9, A, are expressed as mean±SD. On the other hand, Cx43 has been reported to persist at the wound edge epidermis of a majority of mixed diabetic leg ulcers (Brandner, et al. (2004), J Invest Dermatol, vol. 122: 1310-1320).

Human and murine skin punch biopsies analysis showed that Cx43 protein levels were significantly reduced at the wound edge, with levels increasing towards normal with progressive distance from the injury site (FIG. 1, A, B, and C; p<0.01; and FIG. 9, A, arrowheads; p<0.005). In sharp contrast, biopsies from the wound edge of VLU had not only failed to down-regulate Cx43, but also showed significantly elevated expression of Cx43 protein throughout the dermis of the whole 4 mm biopsy taken from chronic VLU in comparison to matched non-wounded samples (FIG. 1, E). The graph shown in FIG. 1, F, depicts the Cx43 protein levels in VLU versus non-wounded skin (p<0.005).

Human and murine skin punch biopsies analysis showed that Cx43 protein levels were significantly reduced at the wound edge, with levels increasing towards normal with progressive distance from the injury site (FIG. 1, A, B, and C; p<0.01; and FIG. 9, A, arrowheads; p<0.005). In sharp contrast, biopsies from the wound edge of VLU had not only failed to down-regulate Cx43, but also showed significantly elevated expression of Cx43 protein throughout the dermis of the whole 4 mm biopsy taken from chronic VLU in comparison to matched non-wounded samples (FIG. 1, E). The graph shown in FIG. 1, F, depicts the Cx43 protein levels in VLU versus non-wounded skin (p<0.005).

Cx43 can interact with adherens junction proteins such as N-cadherin (Wei, et al. (2005), J Biol Chem, vol. 280: 19925-19936; Shaw, et al. (2007), Cell, ol. 128: 547-560), and with the tight junction-associated protein ZO-1 (Giepmans and Moolenaar (1998), Curr Biol, vol. 8: 931-934), which also interact with each other. The expression and distribution of these proteins were investigated in the dermis of chronic VLU biopsies and matched non-wounded skin. ZO-1 expression levels were elevated in the dermis of chronic VLU compared to matched, non-wounded controls (FIG. 2, A; n=6; scale bar=25 μm). Photographs of higher magnifications of VLU and intact skin (boxed regions 1 and 2) stained for ZO-1 (green) and Hoechst (blue) are also shown (FIG. 2, A; scale bar=10 μm.

N-cadherin protein levels were also observed to be significantly up-regulated in chronic VLU as compared to matched, non-wounded samples (FIG. 2, B; n=6; scale bar=25 μm). The boxed regions, numbered 1 and 2, in the far-left photo of FIG. 2, B (scale bar=10 μm), show higher magnifications of VLU and non-wounded skin samples stained with antibodies for N-cadherin (green) and with Hoechst (blue). Scale bar=10 μm.

The graphs in FIG. 2, C and D, show that ZO-1 and N-cadherin protein levels were significantly higher in the dermis of human chronic VLU samples than in matched, non-wounded control dermis. In these graphs, values for ZO-1 and N-cadherin were expressed as mean±SD (p<0.01 and p<0.005, respectively). Taken together, these data indicate that in human chronic VLUs, Cx43 protein levels are not down-regulated in the dermal wound margins, but also that N-cadherin and ZO-1 are abnormally up-regulated compared to their levels in intact skin.

### Cx43 Expression is Tightly Linked to N-Cadherin and ZO-1 Expression in Fibroblasts

It has been previously shown that Cx43 down-regulation significantly accelerates the skin healing process (Qiu, et al. (2003), Curr Biol, vol. 13: 1697-1703; Mori, et al. (2006), J Cell Sci, vol. 119: 5193-5203). In the experiments described in this example, the effects of Cx43 knockdown were investigated on ZO-1 and N-cadherin protein expression in vivo. To this end, an in vivo mouse model of wound healing was used (n=6), where full thickness skin excision wounds were treated with Cx43 antisense or sense control oligodeoxyribonucleotides (Cx43asODN and Cx43sODN, respectively).

Immunohistochemical analysis revealed positive ZO-1 staining in dermal sense-treated wounds, where ZO-1 (green) was frequently co-localized with Cx43 (red) (FIG. 9, B; n=6; scale bar=100 μm). Treatments with Cx43asODN, on the other hand, induced a significant decline not only of Cx43 protein, but also of ZO-1 protein levels in the dermal wound margins (FIG. 9, B; FIG. 3, A, graph represents mean±SD, *p<0.05). In the results shown in FIG. 9, B, the cells also counterstained with Hoechst (blue). These experiments showed a clear down-regulation of ZO-1 protein levels in the dermis of mice treated with the Cx43asODN.

Immunohistochemical analysis of N-cadherin protein expression was also performed, and revealed a preferential distribution of the protein in the dermis in sense-treated wounds and a significant N-cad protein (green) down-regulation after treatment with Cx43asODN as compared to treatment with a control oligonucleotides, Cx43sODN (FIG. 9, C (scale bar=100 μm); FIG. 3, B, graph represents mean±SD, **p<0.01).

Together, these data demonstrate that Cx43 expression is tightly linked to ZO-1 and N-cadherin protein levels in the dermis, and that targeting Cx43 can reduce ZO-1 and N-cadherin expression in vivo.

In order to better understand the effects of Cx43, N-cadherin, and ZO-1 protein levels on fibroblast migration in response to a wounding stimulus, the 3T3 fibroblast cell line was used. This cell line can be easily transfected or transduced with shRNA constructs, and it avoids the variability seen when using primary human fibroblasts derived from different patients. ZO-1 and N-cadherin protein expression was evaluated in fibroblasts transduced with a Cx43 short-hairpin RNA (Cx43shRNA) that suppresses Cx43 expression (van Zeijl, et al. (2007), J Cell Biol, vol. 177: 881-891) or with a p.Sup control plasmid. Cx43 protein expression and gap junction-mediated intercellular communication (GJIC) were effectively prevented in the Cx43shRNA-producing cells, in contrast to p.Sup control cells, as shown in the Western blot and accompanying bar graph in FIG. 3C and in FIG. 10, G, H, and I, which show Cx43 protein levels and dye coupling after LY (Lucifer Yellow) microinjection.

With regard to ZO-1 expression, ZO-1 protein levels were down-regulated in Cx43shRNA-transduced cells (FIG. 3D). ZO-1 distribution was also analyzed in fibroblast scratch wound assays generated with Cx43shRNA and control cells. The Cx43shRNA-transduced cells showed a reduction of ZO-1 located at cell contacts at both the leading edge (LE) and in regions more internal to the wound (IA), accompanied by a loss of ZO-1 from the leading edge lamellipodia (FIG. 3, E, arrowheads). In p.Sup-transduced fibroblasts, however, ZO-1 was predominantly restricted to the contact margins between cells, often co-localizing with Cx43 (FIG. 3, E). With regard to N-cadherin, protein levels were significantly reduced in Cx43shRNA-transduced fibroblasts (FIG. 3, F; p<0.01) and N-cadherin was found located predominantly in the cytosol (FIG. 3, G, arrows) rather than the preferential plasma membrane localization found in p.Sup-transduced control fibroblasts (FIG. 3, G, arrowheads).

The expression and distribution of the proteins α- and β-catenin were also examined in p.Sup and Cx43shRNA-transduced cells. Whereas total β-catenin protein levels did not change significantly after Cx43 knockdown (FIG. 11, B), its cellular distribution was altered. In p.Sup-transduced cells, β-catenin was mainly localized to the plasma membrane at sites of cell-cell contact (FIG. 11, A), but relocation to the cytosol was additionally found in Cx43shRNA-transduced fibroblasts (FIG. 11, A, arrows), with evidence for some nuclear localization (FIG. 11, A, arrowhead). Alpha-catenin was seen localized in both the plasma membrane and the cytosol in p.Sup and Cx43shRNA-transduced cells (FIG. 11, C), and expression levels and location did not change with Cx43 knockdown (FIG. 11, D).

### Cx43 and N-Cadherin, but not ZO-1, Knockdown Accelerate the Rate of Fibroblast Migration

Targeting Cx43 for knockdown accelerates the rate of wound closure in vivo (Qiu, et al. (2003), above). Here, several experiments investigated whether the increase in cell migration following a reduction in Cx43 expression was associated with reduction of ZO-1 protein levels or decreased cell adhesion (ZO-1 or N-cadherin down-regulation, respectively).

Initially, the migration rate of fibroblasts transduced with Cx43shRNA or p.Sup vectors, or cells treated with Cx43asODN or control Cx43sODN, was evaluated. Similar to Cx43shRNA cells, fibroblasts treated with Cx43asODN showed greatly reduced Cx43 protein levels and GJIC compared to Cx43sODN, or untreated cells (FIG. 10, A and B, respectively; p<0.05 and p<0.01). Fibroblasts were also transiently transfected with a bicistronic pIRES-GFP empty vector (pGFP) or with a matching vector encoding GFP and either wild-type Cx43 (Cx43-WT), or a dominant negative Cx43 construct (Cx43-DN) that interfered with the traffic of endogenous Cx43 protein to the plasma membrane, thereby blocking GJIC (Becker, et al. (2001), Cell Commun Adhes, vol. 8: 355-359).

The velocity of migration of Cx43shRNA and Cx43asODN cells was significantly faster than p.Sup or Cx43sODN control cells, respectively (FIG. 4, C and D, p<0.005; FIG. 10, C and D). Cx43 knockdown cells also displayed much more extensive lamellipodia than control cells (FIG. 4C). Similarly, Cx43-DN-transfected fibroblasts migrated faster than their untransfected neighbors or pGFP control cells, and also extended larger than usual lamellipodia at the leading edge (FIG. 10, E and F). Conversely, fibroblasts over-expressing Cx43 (Cx43-WT) migrated significantly slower than either pGFP or Cx43-DN constructs (FIG. 10, E and F; p<0.05 and p<0.01 respectively). These data provide further evidence for an inverse correlation between Cx43 protein levels and the rate of fibroblast migration after scratch wounding. However, there have also been reports that siRNA reduction of Cx43 can slow cell migration in 3T3 cell scratch-wound assays that have been serum starved for 2 days (Francis, et al. (2011), PloS one, vol. 6: e26379).

These experiments were repeated in serum-starved conditions and in the presence of normal amounts of cell culture serum. It was found that in serum-starved conditions, reducing Cx43 protein levels slowed migration, while in more normal media conditions with serum, reducing Cx43 significantly sped migration (FIG. 10, J).

The velocity of migration of fibroblasts transduced with N-cadherin or Mock shRNA constructs (N-cadshRNA and Mock, respectively) was then analyzed. The N-cadshRNA construct effectively suppressed N-cadherin expression in NIH3T3 fibroblasts (FIG. 4, A), which accelerated cell migration in scratch-wound assays almost to the same extent as in Cx43shRNA-transduced cells (FIG. 4, C and D). To reduce ZO-1 expression in fibroblasts, cells were treated with ZO-1 sense and antisense oligodeoxyribonucleotides (ZO-1sODN and ZO-1 asODN), previously described as able to effectively reduce ZO-1 expression (Underwood, et al. (1999), Am J Physiol, vol. 277: C330-342). Contrary to Cx43 and N-cadherin, ZO-1 down-regulation (FIG. 4, B) did not have any effect on the rate of fibroblast migration or wound closure (FIG. 4, C and D). These results indicate that targeting Cx43 may additionally contribute to cell migration by reducing N-cadherin, and perhaps ZO-1, protein levels in fibroblasts.

### Cx43 and N-Cadherin Regulate Cell Adhesion, Polarization and Proliferation in Fibroblasts

Down-regulation of the machinery of cell-cell adhesion is one of the ways in which different cell types, including skin cells, reactivate themselves to acquire a migratory phenotype. Hanging drop assays have previously been used to characterize the strength of intercellular adhesion (Elbert, et al. (2006), Mol Biol Cell, vol. 17: 3345-3355; Redfield, et al. (1997), J Cell Biol, vol. 138: 1323-1331). This approach was thus used to compare cell-cell adhesion properties by measuring the size of cell clusters that formed in suspended drops, and which resisted trituration with a micropipette tip. Clusters were evaluated by morphometric image analysis and classified into three size groups according to their area. Cx43shRNA- and N-cadshRNA-transduced cells formed fewer trituration-resistant clusters (area>2.5×10³ μm²) and smaller clusters (area<7.5×10² μm²) characteristic of reduced cell-cell adhesion (FIG. 5, A and B; p<0.005).

The redistribution of the Golgi apparatus is an important event in the polarization and migration of many types of cells, including fibroblasts (Magdalena, et al. (2003), Mol Biol Cell, vol. 14: 670-684). To examine the role of Cx43 and N-cadherin in cell polarity during cell migration, the localization of the Golgi protein GM130 was analyzed in fibroblasts transduced with Cx43shRNA, N-cadshRNA, or their respective controls. Increased GM130 polarization towards the wound was observed in Cx43shRNA and N-cadshRNA cells compared to p.Sup and Mock controls, 3 h after wounding (FIG. 5, C and D; p<0.05).

Cell proliferation was also investigated using an IncuCyte™ live-cell imaging system. A clear reduction of cell growth was observed in Cx43shRNA and N-cadshRNA transduced cells, compared to p.Sup and Mock control cells (FIG. 5, E and F), indicating a direct correlation between Cx43 and N-cadherin expression, and cell proliferation in fibroblasts.

Altogether these data confirm the important contribution of Cx43 and N-cadherin to the regulation of cell polarization, adhesion, and proliferation processes in fibroblasts during wound repair.

### Targeting Cx43 Induces Cytoskeletal Changes in Leading-Edge Fibroblasts

Using scratch-wound assays it was observed that polarized Cx43 knockdown cells in the wound edge extended larger than usual lamellipodial protrusions. Directional cell locomotion requires complex interactions between actin filaments (F-actin) and microtubules, so the distribution of F-actin, and tyrosinated and acetylated tubulin, was analyzed in wounded monolayers after targeting Cx43 expression in fibroblasts. Front-row control Cx43sODN, pGFP-transfected, and p.Sup fibroblasts displayed the F-actin belt typical of polarized migratory cells (FIGS. 6, A, and 7, A arrowhead; FIG. 12, A), which is regulated by adherens junctions (Yonemura, et al. (1995), J Cell Sci, vol. 108 (Pt 1): 127-142). In contrast, Cx43asODN, Cx43-DN, and Cx43shRNA wound-edge cells developed rich lamelipodial protrusions oriented in the direction of movement, and lacked the F-actin belt found in control cells (FIGS. 6, A, and 7, B arrowhead; FIG. 12, A). Under control conditions, tyrosinated and acetylated microtubules (TyrTub and AcetTub, respectively) were arranged very much as previously described in wounded fibroblast monolayer experiments (Gundersen and Bulinski (1988), Proc Natl Acad Sci USA, vol. 85: 5946-5950), fanning out from the perinuclear region towards the wound margin. In Cx43asODN, Cx43shRNA, and Cx43-DN cells, which tended to be more extended, tyrosinated microtubules were predominantly oriented perpendicularly to the wound edge (FIGS. 6, A, and 7, B arrowhead; FIG. 12, A). Cells over-expressing Cx43 (Cx43-WT) showed microtubules and lamellipodia that were less extensive than pGFP-transfected control cells (FIG. 7, C).

Lamellipodial dynamics were analyzed in more detail by transfecting fibroblasts with a red fluorescent protein (RFP)-actin construct. Sixteen hours after RFP-actin transfection a confluent monolayer was scratch-wounded and imaged for 1.5 hours by confocal microscopy. The p.Sup cells showed active, actin-rich lamellipodia and filopodia (Movie 4) and actin remodeling, characterized by dynamic formation and collapse of filopodia and actin ruffles in front-row cells. Cx43shRNA-transduced fibroblasts displayed considerably more extensive actin-rich membrane protrusions at the front of the leading-edge cells. The extent of cell protrusion was quantified, and it was determined that Cx43asODN, Cx43-DN, and Cx43shRNA cells displayed almost twice the average protrusion length exhibited by Cx43sODN, pGFP, and p.Sup control cells (FIGS. 6, B, and 7, D; FIG. 12, B).

Following demonstration that Cx43 knockdown reduced N-cadherin expression, which also contributes to accelerate cell migration in fibroblasts (FIG. 4,D), the distribution of polymerized actin was analyzed in N-cadshRNA- and Mock-transduced cells 3 h after wound-scratch experiments. Similar to Cx43 knockdown fibroblasts, wound edge N-cadherin-targeted cells extended rich lamellipodial protrusions oriented towards the direction of cell migration (FIG. 13, A), and quantification showed more extensive lamellipodia protrusions compared to Mock control cells (FIG. 13, B).

### Targeting Cx43 and N-Cadherin Increases Rac1 and Rho-A GTPase Activities in Fibroblasts

Rho GTPases are key regulators of actin and microtubule dynamics and play an essential role in controlling different actin-based structures critical for cell motility and chemotactic responses. The effect of Cx43 and N-cadherin knockdown on Rho family GTPase activation in fibroblasts was examined using pull-down assays with specific GST fusion protein-binding domains for activated Rac1, Cdc42, and RhoA (GTP-bound forms). Quantitative analysis showed enhanced Rac1 and RhoA activity in Cx43shRNA and N-cadshRNA cells compared to p.Sup and Mock controls, respectively (FIG. 8, A and B). In contrast, no differences in Cdc42 GTPase activity were detected.

To examine the activation of Rho GTPases in more detail, biosensors for Rac1, RhoA, and Cdc42 (Itoh, et al. (2002), Mol Cell Biol, vol. 22: 6582-6591; Pertz, et al. (2006), Nature, vol. 440: 1069-1072) were transfected into either p.Sup or Cx43shRNA-transduced cells and FRET was used to examine the activity of these GTPases in leading edge fibroblasts 3 hours after scratch-induced migration. Targeting Cx43 with Cx43shRNA induced a two-fold increase in Rac1 and RhoA activity over the p.Sup control (FIG. 8, C and E; p<0.05), but had no effect on the activity of Cdc42 (FIG. 8E). These findings indicate a direct role for Cx43 in the regulation of cytoskeletal dynamics in fibroblasts during cell migration.

### Discussion

Chronic wounds are a significant and growing global problem and place a great burden on both patients and healthcare system resources. While chronic wounds can occur anywhere on the body, most fall within the categories of VLUs, diabetic foot ulcers and pressure ulcers, which fail to progress through an organized, orderly and timely sequence of wound repair. Acceleration or even stimulation of wound closure is important for these chronic wounds, which are often infected and inflamed, making them painful and debilitating for the growing numbers of elderly and diabetic patients, and putting them at risk of lower limb amputation. Here, the experiments show that Cx43 is markedly up-regulated in the dermis of human chronic VLU, similar to what was found in the wound edge epidermis of STZ diabetic rats, a feature that underlies impaired migration and healing (Wang, et al. (2007), Diabetes, vol. 56: 2809-2817).

Because it would be unethical to biopsy and treat human VLU with Cx43 antisense and then resample these hard to heal wounds with additional biopsies, a fibroblast cell line was utilized to reliably manipulate Cx43 protein expression and explore the effects on the dynamics of cell migration in a scratch wound healing assay. Such experiments are useful for exploring the effect of Cx43 modulation on fibroblast behavior. In these cell-culture experiments, fibroblast migration was impaired when Cx43 protein levels and GJIC were elevated after transfection with a Cx43-WT construct. These findings lead to the conclusion that the impaired healing seen in human chronic VLU wounds results from the reduced fibroblast migration rate caused by elevated Cx43 protein levels.

However, elevated levels of Cx43 may not be the only factor that compromises healing in VLU. Cx43 reportedly forms a multiprotein complex or ‘nexus’ with ZO-1, α- and β-catenin, and N-cadherin, proteins that likely influence both cell adhesion and migration in wound healing. In fact, when compared to intact skin, it was found that both ZO-1 and N-cadherin protein levels were significantly up-regulated in the dermis of human chronic VLU, along with Cx43 up-regulation. Clinically relevant is the observation that silencing Cx43 accelerated the velocity of fibroblast migration, which indicates that this process can be therapeutically controlled. Using in vitro models it was recently reported that connexin mimetic peptides also improve the migration rates of dermal fibroblasts (Wright, et al. (2009), Wound Repair Regen, vol. 17: 240-249), as well as keratinocyte and fibroblast migration in organotypic models and 2D cultures (Pollok, et al. (2010), J Cell Mol Med, 2011 Apr; 15(4):861-73), which further reinforce these observations. Those studies reported that levels of Cx43 protein were not changed by the peptide but phosphorylation of Cx43 was increased and cell adhesion decreased. These experiments here, however, revealed that directly targeting Cx43 protein production additionally reduced N-cadherin and ZO-1 protein levels in vivo, with redistribution of both proteins from the plasma membrane to the cytosol. In the case of ZO-1, the protein was noticeably lost from the leading edge lamellipodia, which is normally a feature of fibroblast migration in scratch assay wound healing, which could bring about changes in the distribution of the cytoskeletal components to which it binds.

The cytoplasmic tail of Cx43 is able to interact with the PDZ2 domain on ZO-1, but this interaction can be competed out by the addition of a mimetic peptide (ACT-1) to the last 9 amino acids of the Cx43 tail (Hunter, et al. (2005), Mol Biol Cell, vol. 16: 5686-5698). This peptide has been reported to stabilize Cx43 in the cell membrane forming larger gap junction plaques (Hunter, et al. (2005), Mol Biol Cell, vol. 16: 5686-5698). Applied to acute skin lesions this peptide is reported to accelerate wound healing whilst reducing scar formation, actions that are similar to those of the Cx43 antisense oligo (Gourdie, et al. (2006), Annals of the New York Academy of Sciences, vol 1080: 49-62; Ghatnekar, et al. (2009), Regenerative Medicine, vol 4: 205-223; Rhett, et al. (2008), Trends in Biotechnology, vol. 26: 173-180).

The literature regarding gap junction (GJ) communication and cell migration contains conflicting reports. Elevated connexin expression has been associated with reduced migration (McDonough et al. (1999), Int J Dev Neurosci, vol. 17: 601-611; Batten and Haar (1979), Anat Rec, vol. 194: 125-141), while others have reported the opposite effect (Xu, et al. (2006), Development, vol. 133: 3629-3639; Huang, et al. (1998), J Cell Biol, vol. 143: 1725-1734). Using a different approach, in vitro studies performed on NIH 3T3 cells showed that the dynamic spreading movement, over an hour, of individual isolated and non-wounded cells appeared to be reduced when Cx43 was down-regulated with siRNAs (Wei, et al. (2005), J Biol Chem, vol. 280: 19925-19936). This discrepancy may reflect the different cell states (wounded versus non-wounded) and different models used (cell migration into a scratch-wound as compared to the spreading of non-wounded cells in sparse cultures). More recently, the same group reported that Cx43 KO mouse embryonic fibroblasts migrated more slowly in scratch wound assays than those with Cx43 (Francis, et al. (2011), PloS one, vol. 6: e26379).

The reasons for these divergent observations are not clear, but may be related to serum starving of the cells for 48 hours prior to performing the scratch-wound assay. In the experiments described above, the migration experiments were repeated with control 3T3 cells and 3T3 cells transduced with Cx43shRNA to reduce Cx43 expression. One batch was serum starved for 2 days, while the other was not. Cultures were then scratch-wounded and imaged for 4 hours. It was found that serum starved cells with reduced Cx43 migrated more slowly, whereas reducing Cx43 protein levels in the absence of serum starvation speeded migration. As serum starvation would not be expected to match the conditions found in an acute wound in vivo, where results have shown that down-regulating Cx43 speeds migration of fibroblasts and keratinocytes, it is reasonable to expect that that it is the abnormal conditions of serum starvation that triggered the anomalous response.

Cadherin-mediated cell-cell adhesion is reported to coordinate junction development with cell movement and cell polarization, and to maintain junction integrity by forming links with actin filaments (Pokutta and Weis (2002), Curr Opin Struct Biol, vol. 12: 255-262). Disruption of N-cadherin expression has been reported to increase the rate of Schwann cell migration on astrocytes by enhancing both the number of migrating cells, and the maximum migration distance (Wilby, et al. (1999), Mol Cell Neurosci, vol. 14: 66-84). Here, it was demonstrated that targeting Cx43, which consistently reduced N-cadherin expression, significantly diminished cell-cell adhesion, while increasing cell polarization in fibroblasts. This was further demonstrated by targeting N-cadherin directly, which provided similar results. Direct loss of Cx43-Cx43 connexon docking in Cx43-targeted fibroblasts may also be contribute to decreased cell-cell adhesion, as reported for cadherin null human squamous carcinoma cells (Chakraborty, et al. (2010), J Biol Chem, vol. 285: 10761-10776), and also in other models in which expression of Cx43 increased cell adhesitivity (Lin, et al. (2002), J Neurosci, vol. 22: 4302-4311; Cotrina, et al. (2008), Glia, vol. 56: 1791-1798). In the same way, interfering with Cx43 in individual cells of the 8-16 cell mouse embryo has previously been shown to reduce adhesion and produce decompaction of the targeted cell (Becker and Davies (1995), Microsc Res Tech, vol. 31: 364-374), whereas ZO-1 has recently been reported to be essential for the compaction step (Wang, et al. (2008), Dev Biol, vol. 318: 112-125). Based on the findings described in this Example, it appears that Cx43 down-regulation prevents maturation of stable cell-cell adhesions at least in part by reducing N-cadherin expression and/or docking of connexons themselves.

Here, it has been found that sustained reduction of the expression of either Cx43 or N-cadherin in 3T3 cells, by transduction with shRNA, resulted in reduced cell proliferation. While this observation does not seem to fit with previous reports relating to Cx43 and cell proliferation, it has been found that applying Cx43 antisense to an excisional wound on a mouse results in increased proliferation of fibroblasts and nascent keratinocytes but this was 1, 2, or 7 days after the treatment by which time the antisense was no longer preventing Cx43 protein production (Mori, et al. (2006), J Cell Sci, vol. 119: 5193-5203). Pollock, et al. (2011, above) reported that the mimetic peptide GAP27 could, in some cases, enhance cell proliferation at the leading edge of a scratch-wound assay of keratinocytes or fibroblasts. This effect was brought about without apparently reducing Cx43 protein levels, and so the differences observed when Cx43 protein is significantly reduced likely reflect the direct effect of the presence of the Cx43 protein on cell proliferation rather than the effect of Cx43-based gap junctional communication.

Trafficking of Cx43 hemichannels to cell-cell junctions takes place through a pathway that is dependent on microtubules, and it is known that the cytoplasmic tail of Cx43 is able to interact with various components of the cytoskeleton, including microtubules and actin. Here, the experiments described above show that front-row migrating fibroblasts lacking N-cadherin or Cx43 protein adopt an elongated phenotype with lamellipodia extended into the wound bed in the direction of migration. In contrast, actin organization in control-wounded fibroblasts was characterized by the F-actin belt typical of polarized migratory cells, known to be regulated by adherens junctions. Enhanced Rac1 and RhoA activities were also identified in Cx43 and N-cadherin-targeted fibroblasts. These findings correlate with a reported role for RhoA in regulating fibroblast protrusion as an initiator of actin polymerization at the onset of the protrusion-retraction cycle (Machacek, et al. (2009) Nature, vol. 461: 99-103). Rac1, on the other hand, can influence the reinforcement and stabilization of newly expanded protrusions, which may explain the faster migration and more extensive lamellipodia observed in Cx43-targeted fibroblasts.

The organization of the microtubule cytoskeleton was also investigated and found to be altered after reducing Cx43 protein levels, indicating that Cx43 not only interacts directly with microtubules, but may also affect microtubule dynamics during fibroblast migration. See Francis, et al. (2011), PloS one, vol. 6: e26379, reporting altered microtubule dynamics in Cx43 KO mouse embryonic fibroblasts).

Overall, the experiments described in this Example provide insight into why chronic VLUs that overexpress Cx43 and cadherin in the dermis are slow to heal, and the cellular mechanisms as to how reducing Cx43 and cadherin protein expression accelerates fibroblast migration, pointing to a therapeutic solution to this debilitating problem. These findings support the role of Cx43 in not simply forming GJ channels, but also to stabilize a nexus or multiprotein complex comprising N-cadherin, amongst others, that is required for cell-cell adhesion and adhesion-dependent actin dynamics, which must be broken down to facilitate efficient fibroblast migration.

### Example 3

### Cx43, N-Cad, and ZO-1 Overexpression In Diabetic Foot Ulcers Retards Fibroblast Migration

### Introduction

Poor healing of diabetic foot ulcers is a major clinical problem that can be extremely debilitating and lead to lower limb amputation. In normal acute wounds, the Connexin 43 (Cx43) gap junction protein is down-regulated at the wound edge as a precursor to cell migration and healing. In this example, experiments that use fibroblasts from the human chronic diabetic foot ulcer wound edge show a striking and significant 10-fold elevation of Connexin 43 protein, as well as a 6-fold increase in N-cadherin (N-Cad) and a 2-fold increase in Zonular Occludin-1 (ZO-1, a zona occludens protein), as compared to unwounded skin. In streptozotocin diabetic rats, Cx43 was found to be upregulated in intact dermal fibroblasts in direct proportion to blood glucose levels and increased 2-fold further in response to wounding. To mimic diabetes, 3T3 fibroblasts were cultured under different concentrations of glucose or mannitol and Cx43 protein intercellular communication and migration rates were determined Cultures of fibroblasts in very high (40 mM) glucose conditions showed significantly elevated Cx43 protein levels, as shown by immunostaining and Western Blot, and significantly increasing gap junctional communication, as shown by dye transfer. In scratch wound healing assays, increased levels of Cx43 from high glucose resulted in repressed filopodial extensions and significantly slower migration rates than in either standard conditions (5.5 mM glucose) or the osmotic control of mannitol. Conversely, when glucose-induced Connexin 43 upregulation was prevented with Cx43shRNA transduction, the fibroblasts extended long filopodia and migrated significantly faster. Connexin 43 protein was upregulated in fibroblasts in diabetic foot ulcers as well as after high glucose exposure in culture which correlated with inhibition of fibroblast migration and is likely to contribute to impaired wound healing.

### Background

People with diabetes can have wounds that heal poorly and suffer a high incidence of non-healing skin ulcers, most frequently found on the feet. Such ulcers can be extremely debilitating, and a significant number eventually lead to lower limb amputations.

The gap junction protein Connexin 43 (Cx43) plays a central role in the wound healing response. In wound edge keratinocytes, Cx43 is normally down-regulated in the first 24-48 hours after injury, as keratinocytes become migratory and crawl forward to heal the wound. An accelerated transition to the migratory state in rodents has been shown when Cx43 is more rapidly down-regulated by application of a Cx43 specific antisense gel to the wound. In contrast, the Cx43 protein in Streptozotocin (STZ) diabetic rats is abnormally over-produced in wound edge keratinocytes after wounding and migration fails to occur until Cx43 protein levels are reduced. Abnormal Cx43 protein expression is, at least in part, believed to underpin the poor healing observed in diabetic skin ulcers, and recovery of normal migratory rates of keratinocytes in STZ diabetic rats can be achieved by application of Cx43 antisense to wounds, a treatment that prevents the abnormal elevation of Cx43.

In the experiments described below, the dermal Cx43 protein levels have been quantified in intact human skin biopsies taken from normoglycemic donors, and from forearm and ulcer wound edge biopsies in patients with confirmed diabetes. Complementary in vivo and in vitro studies are described in which the effects of elevated glucose on Cx43 protein levels, gap junctional communication, and migration in a scratch wound-healing assay, as well as the contribution of Cx43 protein levels to the rate of migration, were examined

### Materials and Methods

2.1 Human Skin Biopsies

The collection of 4 mm punch biopsies from the edges of chronic diabetic foot ulcers was approved by the Western Institutional Review Board, Olympia, Washington State, USA. This sample was taken from the ulcer wound edge as well as a matched forearm biopsy of unwounded skin. A separate non-diabetic cohort had a biopsy of forearm skin. All biopsies were taken after written informed consent was obtained.

The diabetic cohort comprised 10 Caucasian subjects (6 males and 4 females) with median age 59.5 yrs (range: 48-82 yrs). Nine subjects were on insulin and one on oral medication, with the cohort median HBA1c 6.9 (range 5.3-9.9; n=6 available). All had diabetic neuropathy of the extremities and 6 had clinical indications of peripheral vascular disease. Each subject had a clinically confirmed diabetic foot ulcer. Median ulcer size 9.4 cm² (1-81 cm².) with a duration median of 3.5 mths (range 1.5-26 mths). The control cohort of non-diabetic intact skin arm biopsies was taken from Caucasian individuals, comprising 6 subjects of median age 48.5 years (2 males and 4 females; range 36-79 years.

### Diabetic Rat

Diabetes was induced in male Sprague-Dawley rats (350-400 g) by intraperitoneal injection of STZ (65 mg/Kg) and diabetes confirmed using glucose urinary strips (Clinistix, Bayer, UK). Wound healing studies were undertaken 2 weeks after induction of diabetes and were performed as previously described (Wang, Lincoln (2007), Diabetes, vol. 56:2809-17). A single topical application of 50 μl of 10 μM unmodified Cx43asODNs (5′-GTA ATT GCG GCA GGA GGA ATT GTT TCT GTC-3′; SEQ ID NO:10) or control Cx43sODNs (5′-GAC AGA AAC AAT TCC TCC TGC CGC AAT TAC; SEQ ID NO:11; Sigma) was delivered to wounds and tissue harvested at 24 hours after wounding. Blood glucose readings were taken and all rats were confirmed to be severely hyperglycaemic (blood glucose 27.07±1.09 mmol/L).

### Cell Culture

Confluent monolayers of NIH 3T3 fibroblasts were grown in 5.5% glucose DMEM (D5796: Invitrogen, UK) supplemented 10% fetal bovine serum and penicillin/streptomycin. In some experiments the level of glucose was elevated to 25 or 40 mM or an osmotic control of 19.5 or 34.5 mM mannitol. Cells were cultured in these conditions for 2 weeks prior to the experiments being performed. Dye-injection was carried out according to the method described by Becker, et al. (Journal of Cell Science (1995), vol. 108, part 4:1455-67). The scratch wound assay was performed on confluent monolayers of fibroblasts as described by Mori, et al. (Journal of Cell Science (2006(, vol. 119:5193-203), and migration was monitored by time lapse imaging over a 4 hour period on an Olympus IX81 microscope.

### Immunohistochemistry and Imaging

Immunostaining was carried out on cultured cells or cryostat sections of wounded and intact skin as described by Wang, et al. (Diabetes (2007), vol. 56:2809-17). Primary antibodies for Cx43, diluted 1:2000 (Sigma, Poole, UK), N-cadherin, diluted 1:100 (Abcam, ab18203), and ZO-1, diluted 1:100 (Zymed Laboratories, 61-7300), were incubated for 1 hour at room temperature. Tissues were washed with PBS and then incubated with the secondary antibody swine anti-rabbit FITC-conjugated (DAKO). Secondary antibody incubation in the absence of primary antibody was used as negative control. Tissues were then counterstained for 10 minutes with 5 μg/ml of the nuclear dye Hoechst 33342 (Sigma, Poole, UK) and images were acquired on a Leica SP2 confocal microscope (Leica Microsystems, UK). All parameters during image acquisition were kept constant throughout each experiment to allow direct comparison of all of the 8 bit digital images. When imaging, major dermal appendages were avoided in the field of view as they express considerably more Cx43 and would have distorted the results Immunostaining levels were quantified per unit area using a well-established pixel-counting method using ImageJ software (NIH). Images were converted to binary images using an identical threshold. Objects greater than 2 pixels were counted in order to generate a readout of the number of positive pixels per unit area for comparison between conditions.

Retroviral Constructs: A retroviral pSuper vector contained the Cx43shRNA sequence 5′-GGTGTGGCTGTCAGTGCT-3′ (SEQ ID NO:12; van Zeijl, et al. (2007), J. Cell Biol., vol. 177:881-91). A pSuppressor (p.Sup) retroviral vector acted as control, and transfection was performed as described by Carr and Whitmore (Nature Cell biology (2005), vol. 7:319-21).

### Statistical Analysis

Statistical differences were determined using ANOVA followed by Tukey's analysis with P<0.05 taken as significant.

### Results

Cx43, N-Cadherin and ZO-1 Protein Levels in Human Biopsies

The effect of the underlying diabetic state on the human fibroblast Cx43 protein levels from intact non-wounded dermis was negligible in terms of Cx43 protein levels in contrast to the effects of the diabetic foot ulcer (DFU). Fibroblasts from within DFU showed a striking upregulation of Cx43 protein, which was about 10-fold higher than the comparable intact diabetic or non-diabetic skin (P<0.05). Cx43 protein is often closely associated with the tight junction protein ZO-1 and the adhesion protein N-Cadherin, and both of these proteins were also found to be elevated in the DFU samples. N-Cadherin protein levels increased 6-fold in DFU compared to intact skin (P<0.05), and mean ZO-1 protein levels were found to be elevated 2-fold. It was also observed that in the intact human skin, autofluorescent bundles of elastin could be seen (but are not seen in rat skin), and these were absent from the wound edge of the DFU where they have been degraded by proteases.

Cx43 Protein Levels in Wound Edge Fibroblasts of STZ Rats

Cx43 immunostaining of dermal fibroblasts in the wound-edge region of STZ diabetic rats was more than double that in control rats 24 hours after wounding. This abnormal increase of Cx43 protein in diabetic wound-edge fibroblasts was efficiently prevented (P<0.001) by a single topical application of a Cx43-specific antisense gel (Cx43asODNs; SEQ ID NO:13) immediately after wounding. In the intact diabetic rat skin, there was found to be a significant increase in Cx43 protein levels in direct relation to blood glucose levels r=0.625.

Fibroblast Cx43 Expression and Communication

3T3 fibroblasts transduced with p.Sup or Cx43shRNA constructs were cultured under increasing glucose (5.5, 25 and 40 mM) conditions for 2 weeks in order to mimic the diabetic state in these cells. To control for effects of increased glucose osmolarity, fibroblasts were also incubated in 5.5 mM glucose DMEM supplemented with 19.5 or 34.5 mM mannitol, respectively. The 40 mM levels of glucose resulted in significantly elevated levels of Cx43 protein, as shown by Western blotting and immunostaining (P<0.01). This elevation in Cx43 protein levels was largely prevented by transduction with Cx43shRNA (P<0.001). The extent of cell communication was analyzed in fibroblasts cultured in different concentrations of glucose and a significant increase in the incidence of dye coupling was seen at the highest glucose concentration (40 mM) as compared with lower glucose or control high mannitol conditions (P<0.01).

Fibroblast Migration

Cells cultured under increasing glucose conditions for two weeks migrated significantly slower in response to a scratch wound when imaged over a 4 hour period (P<0.05 and P<0.001 for 25 and 40 mM glucose, respectively) than 5.5 mM glucose. There were no significant changes in the rate of migration for fibroblasts incubated with 19.5 mM and 34.5 mM mannitol, ruling out osmotic effects. When fibroblasts stably transduced with Cx43shRNA or p.Sup were incubated under increasing glucose and mannitol conditions, the Cx43shRNA largely prevented the Cx43 upregulation seen in the 40 mM glucose conditions and significantly enhanced the migration rates for all conditions (P<0.05; P<0.001). Although the velocity of migration for the Cx43shRNA 40 mM high-glucose dose increased 2-fold over p.Sup 40 mM glucose, it did not reach the velocity seen in all of the other Cx43shRNA treatment conditions. However, it did match or exceed the rates obtained in all p.Sup-transfected fibroblasts culture. In addition, Cx43shRNA also enhanced the rate of production and the size of lamellipodial extensions at the leading edge of the migrating cells consistent with a more rapid onset of migration and greater motility.

### Discussion

In this study, for the first time a striking 10-fold increase has been observed in the expression level of the gap junction protein Cx43 quantified in dermal fibroblasts from biopsies of human DFU wound edges. It is believed that this abnormal expression of Cx43, a protein that normally must be transiently down-regulated in acute wound healing, inhibits the ability of the fibroblasts to migrate and heal such chronic wounds.

These results show that elevation of Cx43 occurs in wound edge fibroblasts in STZ rats, and that this can be prevented by a Cx43asODN. The increase in Cx43 in the intact STZ rat dermis was found to be in direct proportion to the level of blood glucose, so glucose itself may at least in part be a driver of Cx43 expression changes in fibroblasts. Similarly, these results also show that raising the level of glucose to 40 mM in the media of 3T3 cell cultures elevated Cx43 protein levels and GJIC. This effect was brought about by glucose, not increased osmolarity, as similar levels of mannitol did not significantly increase Cx43 protein or GJIC. The elevated levels of Cx43 had a negative effect on the migration rate of fibroblasts, which is consistent with the perturbed wound healing of STZ diabetic rats that show abnormally elevated Cx43 in wound edges. Strong supportive evidence that elevated levels of Cx43 retard fibroblast migration was additionally provided by demonstrating that cells migrated at control rates or faster after Cx43 knockdown. Even at very high glucose levels (40 mM) the Cx43shRNA migration was 2-fold faster than p.Sup (40 mM) and in excess of the level seen at a p.Sup control (5.5 mM) glucose concentration. While Cx43shRNA could easily prevent expression of normal levels of Cx43 protein, it did not entirely prevent the elevated levels induced by 40 mM glucose. Interestingly, when Cx43shRNA (40 mM glucose) Cx43 levels are similar to those of p.Sup (5.5 mM glucose), the migration rates are also similar.

The importance of down-regulation of Cx43 protein during the wound healing process is highlighted by the fact that fibroblast migration is impaired if Cx43 protein levels are elevated. It would appear that the more Cx43 protein that is present, the slower fibroblasts migrate. The 10-fold elevation of Cx43 protein in fibroblasts of DFU may explain why these cells fail to migrate. Precisely how Cx43 inhibits fibroblast migration is not yet clear; however, the cytoplasmic tail of Cx43 can interact with a number of cytoskeletal and membrane proteins such as α- and β-catenin, N-cadherin, and ZO-1, and can form a multi-protein junctional complex sometimes referred to as a “Proteome” or “nexus”, which may affect migration. In addition, the Cx43 gene may be a master gene that controls the expression of other genes. The discovery here that both ZO-1 and N-cadherin are upregulated in the dermis of human diabetic skin, and even more in DFU, also supports a regulatory role for Cx43. The increase in adhesion generated by elevated N-cadherin protein levels may contribute to the retarded fibroblast migration. Additionally, cell-cell adhesion will also increase by virtue of the elevated Cx43-Cx43 hemichannel docking between DFU fibroblasts.

### Summary and Conclusions

In this study 10-fold elevations of Cx43 protein levels were found in dermal fibroblasts of human biopsies from diabetic foot ulcers. Additionally, the levels of N-cadherin and ZO-1 were discovered to be elevated 6- and 2-fold, respectively, in DFU. It was also shown that Cx43 was elevated in the STZ diabetic rat dermis in proportion to the level of blood glucose, and that the observed 3-fold elevation of Cx43 in wound edge dermis of STZ diabetic rats could be corrected by application of Cx43-specific antisense to the wound. It was also shown that in cultured fibroblasts high levels of glucose can induce elevated Cx43 protein levels, and that such glucose levels retard fibroblast migration in vitro. These results confirm that increased Cx43 expression is a fundamental cause of poor fibroblast migration and reduced healing rates in diabetic ulcers.

### Example 4

### Oligonucleotides Targeting ZO-1

This Example describes several candidates for ZO-1 AS ODNs (antisense oligodeoxtribonucleotides), shRNAs (small hairpin RNA molecules), and siRNAs (small interfering RNA molecules).

ZO-1 was originally been identified at tight junctions, which form a network inside cells. This structure is only present at the intersection between two cells at the cell-cell contact zone. ZO-1 is a 220-kDa membrane protein, which co-localizes with the transmembrane proteins claudins and occludin. Later, ZO-1 was demonstrated and identified at adherens junctions that zip cells together and thereby maintain cell and tissue polarity. These junctions also anchor the cytoskeleton, allowing the formation of large complexes at the plasma membrane.

One sequence selected for synthesis of antisense polynucleotides that target ZO-1,5′-CTGCTTTCTGTTGAGAGGCT-3′ (SEQ ID NO:14), corresponds to the segment from base pair 3154-3169 in MUSZO1 accession number D143401. The complementary sense polynucleotide, 5′-AGCCTCTCAACAGAAAGCAG-3′ (SEQ ID NO: 25), and a random-order nonsense polynucleotide, 5′-TATGGTACGTGTCGTCCTTG-3′ (SEQ ID NO: 26), can be used as controls.

Small interfering RNAs (whether now known or later developed) can also be used to reduce or eliminate ZO-1 expression. Several such molecules include:

siRNA scrambled
(SEQ ID NO: 15)
5′-GGGAAGACAGAACUUGUACUCAAAA-3′
(SEQ ID NO: 16)
3′-CCCUUCUGUCUUGAACAUGAGUUUU-5′
siRNA p53
(SEQ ID NO: 17)
5′-AAAACUCAUGUUCAAGACAGAAGGGU-3′
(SEQ ID NO: 18)
3′-UUUUGAGUACAAGUUCUGUCUUCCCA-5′
siRNA ZO-1 1681
(SEQ ID NO: 19)
5′-CCAUCUGAUGGUGUCCUACCUAAUU-3′
(SEQ ID NO20_)
3′-GGUAGACUACCACAGGAUGGAUUAA-5′
siRNA ZO-1 2137
(SEQ ID NO: 21)
5′-GGGCUCUUGGCUUGCUAUUCGAAUU-3′
(SEQ ID NO: 22)
3′-CCCGAGAACCGAACGAUAAGCUJAA-5′
siRNA ZO-1 5518
(SEQ ID NO: 23)
5′-CCUUCCACCUUUAGAUAAAGAGAAA-3′
(SEQ ID NO: 24)
3′-GGAAGGUGGAAAUGUAUUUCUCUUU-5′

All patents, publications, scientific articles, web sites, and other documents and materials referenced or mentioned herein are indicative of the levels of skill of those skilled in the art to which the invention pertains, and each such referenced document and material is hereby incorporated by reference to the same extent as if it had been incorporated by reference in its entirety individually or set forth herein in its entirety. Applicants reserve the right to physically incorporate into this specification any and all materials and information from any such patents, publications, scientific articles, web sites, electronically available information, and other referenced materials or documents.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “consisting essentially of”, and “consisting of may be replaced with either of the other two terms in the specification. Also, the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also that as used herein and in the appended claims, the singular forms “a,” “an,” and “the” include plural reference unless the context clearly dictates otherwise. Under no circumstances may the patent be interpreted to be limited to the specific examples or embodiments or methods specifically disclosed herein. Under no circumstances may the patent be interpreted to be limited by any statement made by any examiner or other official or employee of government patent office unless such statement is specifically and without qualification or reservation expressly adopted in a responsive writing by or on behalf of the inventor(s).

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

We claim:

1. A polynucleotide, optionally an antisense polynucleotide, an RNA, shRNA, miRNA or an siRNA, to a messenger RNA (mRNA) encoding a protein that is a member of the cadherin protein superfamily, optionally a human cadherin protein, optionally a cadherin protein selected from the group consisting of N-cadherin, E-cadherin, P-cadherin, cadherin 11, cadherin 12, a protocadherin protein, a desmoglein protein, and a desmocollin protein, wherein the polynucleotide optionally is between about 5 and about 100 nucleotides in length, optionally about 6 to about 40 nucleotides in length.

2. A polynucleotide according to claim 1 that is an oligodeoxynucleotide, optionally an unmodified phosphodiester oligodeoxynucleotide or a chemically modified deoxyoligonucleotide, optionally a chemically modified oligonucleotide selected from the group consisting of phosphorothioates, methylphosphonates, phosphoramidates, phosphorodithioates, N3′P5′-phosphoramidates, oligoribonucleotide phosphorothioates and their 2′-O-alkyl analogs, 2′-O-methylribonucleotide methylphosphonates, and mixed backbone oligonucleotides.

3. A polynucleotide according to claim 1 that binds a target nucleotide sequence in an N-cadherin gene open reading frame, wherein the target nucleotide sequence comprises fewer than about 100 nucleotides that includes the sequence 5′-GACTGGATTTCCTGAAGAT-3′ (SEQ ID NO:7), or an RNA equivalent thereof.

4. A polynucleotide according to claim 3 that is an antisense polynucleotide, an RNA, shRNA, or an siRNA, wherein the polynucleotide optionally has at least about 70%, 80%, 90%, 95%, or 100% complementarity to an mRNA or a portion of the messenger RNA that comprises the target nucleotide sequence.

5. A use of at least one antisense polynucleotide according to claim 1 in the manufacture of a medicament for use in treating a disease or condition correlated with aberrant or undesired cadherin activity, wherein the disease or condition optionally is selected from the group consisting of an acute wound, a chronic wound, an inflammatory disease, a lung diseases (optionally asthma), a renal disease, a liver diseases (optionally NASH), arthritis (optionally juvenile arthritis, osteoarthritis, and rheumatoid arthritis), an inflammatory bowel disease (optionally Crohn's disease and ulcerative colitis), a dermatosis, an infection, ischemia (optionally a reperfusion injury), and a cardiac disease (optionally atherosclerosis).

6. A composition comprising at least one antisense polynucleotide according to claim 1 and a physiologically acceptable carrier or vehicle, wherein composition optionally is formulated for administration via a route selected from the group consisting oral, administration, topical administration, and injection.

7. A composition according to claim 6 which is in the form of a cream, ointment, gel, emulsion, lotion, foam, or paint, wherein when the composition is a gel, the gel optionally comprises a nonionic polyoxyethylene-polyoxypropylene copolymer gel.

8. A composition according claim 6 that further includes a surfactant or urea to assist with polynucleotide penetration into cells.

9. A method of delivery of an antisense polynucleotide to a messenger RNA (mRNA) encoding a protein that is a member of the cadherin protein superfamily, comprising administering to a subject, optionally a human or non-human animal, optionally a mammal, in need of treatment with a modulator of a said protein a composition according to claim 6, thereby delivering an antisense polynucleotide to a mRNA encoding said protein.

10. A method according to claim 9 wherein the subject suffers from a wound, optionally a wound selected from the group consisting of an acute wound, a delayed-healing wound, an incompletely healing wound, a chronic wound (optionally a diabetic ulcer, a venous ulcer, a pressure ulcer, a vasculitic ulcer, or an arterial ulcer), and a dehiscent wound, and wherein the composition optionally is applied prior to repair or closure of a wound.

11. A method of decreasing in a subject expression of a protein that is a member of the cadherin protein superfamily, comprising administering to the subject a composition according to claim 6, thereby decreasing expression of said protein.

12. A method of treatment comprising administering to a subject in need thereof a composition comprising therapeutically effective amounts of a first wound-healing agent and a second wound-healing agent, wherein said first wound-healing agent is a antisense polynucleotide according to claim 1 and the second agent is selected from the group consisting of an anti-connexin 43 polynucleotide, an anti-connexin 43 peptide, or peptidomimetic, a hemichannel closing or blocking agent, a connexin 43 carboxy-terminal polypeptide gap junction closing or blocking agent, and an anti-osteopontin polynucleotide.

13. A method according to claim 12 wherein said first and second wound-healing agents are administered in combination, optionally administered separately, optionally at about the same time or sequentially.

14. A method according to claim 12 wherein the first or second wound-healing agent is administered first.

15. A method according to claim 12 wherein said first and second wound-healing agents are administered within about 1 to about 7 days of each other, optionally within about 1 to about 2 days of each other, optionally within about 6 to about 24 hours of each other, optionally within about 1 to about 6 hours of each other.

16. An article of manufacture comprising package material containing a polynucleotide according to claim 1 together with instructions for use.
